(19) United States
(12) Patent Application Publication
(10) Pub. No.: US 2025/0032514 A1
(43) Pub. Date: Jan. 30, 2025

(54) COMPOSITIONS AND METHODS FOR THE TREATMENT OF VARIOUS DISEASES

(71) Applicant: Amylyx Pharmaceuticals, Inc., Cambridge, MA (US)

(72) Inventors: Joshua Barry Cohen, Canton, MA (US); Justin Bernard Klee, Cambridge, MA (US); Mathias Leinders, Laguna Beach, CA (US); Lahar Mehta, Redmond, WA (US)

(21) Appl. No.: 18/780,691

(22) Filed: Jul. 23, 2024

Related U.S. Application Data
(60) Provisional application No. 63/528,900, filed on Jul. 25, 2023.

(51) Int. Cl. A61K 31/575; A61K 31/192; A61P 25/28

(52) U.S. Cl. CPC A61K 31/575; A61K 31/192; A61P 25/28

(57) ABSTRACT

Provided herein are methods and compositions for treating a neurodegenerative disease (e.g., PSP). The methods can include administering to the subject a bile acid or a pharmaceutically acceptable salt thereof and a phenylbutyrate compound.

### CROSS-REFERENCE TO RELATED APPLICATIONS

This application claims the benefit of priority to U.S. Application No. 63/528,900, filed on Jul. 25, 2023, the contents of which are hereby incorporated by reference.

### TECHNICAL FIELD

The present disclosure generally relates to compositions and methods for treating various disorders.

### BACKGROUND

Progressive supranuclear palsy (PSP) is a tauopathy characterized by degeneration in the cerebral cortex, basal ganglia, and brainstem. It is associated with abnormal tau protein folding and aggregation, in particular the 4R tau isoform. The disease is characterized by progressive aberrations in gait, eye movements, dysphagia, dysarthria, pseudobulbar affect, neuropsychiatric abnormalities, dementia, and is ultimately fatal. Mean survival from symptom onset is approximately 6 to 8.5 years (Stamelou 2021).

PSP has an estimated prevalence of 5 to 17.9 cases per 100,000 people depending on geography (Coyle-Gilchrist 2016, Takigawa 2016). Symptom onset is most often between the ages of 50 to 70 years. Though some heterogeneity in clinical presentation exists, the most common and initially described PSP syndrome, now referred to as Steele-Richardson-Olszewski Syndrome (PSP—RS), presents with symptoms including early postural instability and oculomotor dysfunction, including slow vertical saccades and supranuclear gaze palsy leading to falls.

There is currently no treatment available to stop or slow the progression of this deadly disease. In the absence of any effective disease-modifying or neuroprotective therapies, PSP represents an urgent unmet medical need.

### SUMMARY

Accordingly, in some embodiments, the present disclosure provides methods of treating at least one symptom of progressive supranuclear palsy (PSP) in a human subject, the method comprising administering to the human subject a pharmaceutically effective amount of a combination of taurursodiol (TURSO) and sodium phenylbutyrate. In some aspects, the present disclosure provides methods of treating a human subject having progressive supranuclear palsy (PSP), the method comprising administering to the human subject a pharmaceutically effective amount of a combination of TURSO and sodium phenylbutyrate.

In some embodiments, provided herein are methods of treating at least one symptom of progressive supranuclear palsy (PSP) in a human subject, the method comprising administering to the human subject a pharmaceutically effective amount of a combination of TURSO and sodium phenylbutyrate.

In some embodiments, provided herein are methods of treating a human subject having progressive supranuclear palsy (PSP), the method comprising administering to the human subject a pharmaceutically effective amount of a combination of TURSO and sodium phenylbutyrate.

In some embodiments, provided herein are methods of slowing progressive supranuclear palsy (PSP) progression in a human subject having one or more symptoms of PSP, the method comprising administering to the human subject a pharmaceutically effective amount of a combination of TURSO and sodium phenylbutyrate.

In some embodiments, provided herein are methods of increasing survival time of a human subject having one or more symptoms of progressive supranuclear palsy (PSP), the method comprising administering to the human subject a pharmaceutically effective amount of a combination of TURSO and sodium phenylbutyrate.

In some embodiments, provided herein are methods of decreasing the level of total CSF tau, decreasing the level of CSF phospho-tau, increasing CSF Aβ₁₋₄₂/Aβ₁₋₄₀, or increasing the level of CSF 8-OHDG in a human subject having one or more symptoms of progressive supranuclear palsy (PSP), the method comprising administering to the human subject a pharmaceutically effective amount of a combination of TURSO and sodium phenylbutyrate. In some aspects, the phospho-tau species is phospho-tau 181.

In some embodiments, provided herein are methods comprising administering to a human subject at risk for developing progressive supranuclear palsy (PSP) a pharmaceutically effective amount of a combination of TURSO and sodium phenylbutyrate. In some aspects, the subject is determined to be at risk for developing progressive supranuclear palsy (PSP) by evaluating a level of a biomarker in a biological sample obtained from the subject. In some aspects, the biomarker is total tau or phospho-tau. In some aspects, the biological sample is plasma or CSF.

In some embodiments, provided herein are methods of decreasing the level of CSF YKL-40, decreasing the level of Ptpn1, or increasing the CSF ratio of 33 kDa tau to 55 kDa tau in a human subject having one or more symptoms of PSP, the method comprising administering to the human subject a pharmaceutically effective amount of a combination of TURSO and sodium phenylbutyrate.

In some embodiments of any one of the above-described embodiments, the human subject:
(a) has possible or probable PSP (Steele-Richardson-Olszewski Syndrome) according to MDS 2017 criteria;
(b) has had PSP symptoms for less than 5 years;
(c) has a PSP Rating Score total score of less than 40; or
(d) has a Mini-Mental Score Exam of greater than or equal to 24. In some aspects, the method comprises, prior to administration, a step of determining whether the human subject has at least one of the characteristics of (a)-(d).

In some embodiments of any one of the above-described embodiments, wherein the TURSO and the sodium phenylbutyrate are administered once a day or twice a day.

In some embodiments of any one of the above-described embodiments, wherein TURSO is administered to the subject at a dose of about 5 mg/kg to about 100 mg/kg.

In some embodiments of any one of the above-described embodiments, wherein sodium phenylbutyrate is administered to the subject at a dose of about 10 mg/kg to about 400 mg/kg.

In some embodiments of any one of the above-described embodiments, wherein the TURSO is administered at an amount of about 0.5 to about 5 grams per day.

In some embodiments of any one of the above-described embodiments, wherein the sodium phenylbutyrate is administered at an amount of about 0.5 grams to about 10 grams per day.

In some embodiments of any one of the above-described embodiments, the methods comprise administering to the subject about 1 gram of TURSO and about 3 grams of sodium phenylbutyrate once a day or twice a day.

In some embodiments of any one of the above-described embodiments, the methods comprise administering to the subject about 1 gram of TURSO once a day and about 3 grams of sodium phenylbutyrate once a day for about 14 days or more, followed by administering to the subject about 1 gram of TURSO twice a day and about 3 grams of sodium phenylbutyrate twice a day.

In some embodiments of any one of the above-described embodiments, the methods further comprise assessing one or more outcome measure after about 24 weeks of administering TURSO and sodium phenylbutyrate to the subject, determining an improvement in one or more of the outcome measures, and continuing administration of TURSO and sodium phenylbutyrate. In some aspects, the outcome measure is Progressive Supranuclear Palsy (PSP) Rating Score, Movement Disorder Society-Unified Parkinson's Disease Rating Scale Part II, Schwab and England Activities of Daily Living Scale, EuroQuality of Life, Zarit Burden Interview, Clinical Global Impression of Severity and Change, Mini-Mental State Exam, Montreal Cognitive Assessment, Columbia-Suicide Severity Rating Scale, one or more plasma biomarker, or one or more cerebriospinal fluid (CSF) biomarker.

Also provided herein are methods of treating or improving at least one symptom of progressive supranuclear palsy (PSP) in a human subject, the method comprising: assessing a baseline PSP Rating Scale score prior to initial administration of TURSO and sodium phenylbutyrate to the human subject, administering to the human subject about 1 gram of taurursodiol (TURSO) once a day and about 3 grams of sodium phenylbutyrate once a day for at least three weeks, followed by administering to the human subject about 1 gram of TURSO twice a day and about 3 grams of sodium phenylbutyrate twice a day, assessing a second PSP Rating Scale at about 24 weeks after initial administration of TURSO and sodium phenylbutyrate to the human subject, determining that the second PSP Rating Scale is higher than the baseline PSP Rating Scale, and continuing to administer to the human subject about 1 gram of TURSO twice a day and about 3 grams of sodium phenylbutyrate twice a day, thereby treating or improving at least one symptom of progressive supranuclear palsy (PSP).

In some embodiments of any one of the above-described embodiments, the TURSO and the sodium phenylbutyrate are administered orally.

In some embodiments of any one of the above-described embodiments, the TURSO and the sodium phenylbutyrate are formulated as a single powder formulation.

In some embodiments of any one of the above-described embodiments, the method further comprises administering one or more additional therapeutic agents to the subject.

In some embodiments of any one of the above-described embodiments, the method further comprises administering to the human subject a plurality of food items comprising solid foods or liquid foods.

In some embodiments of any one of the above-described embodiments, the human subject is about 18 years or older.

In some embodiments of any one of the above-described embodiments, the human subject is about 40-80 years.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

### BRIEF DESCRIPTION OF THE DRAWINGS

FIG. 1 is a schematic of the study design.

### DETAILED DESCRIPTION

A major class of neurodegenerative diseases, collectively known as tauopathies, are characterized by intra-cellular inclusions composed of abnormally modified microtubule-binding protein, tau, at autopsy. A tauopathy is a disorder characterized by an abnormal level of tau in a cell, a tissue, or a fluid in an individual. In some cases, a tauopathy is characterized by the presence in a cell, a tissue, or a fluid of elevated (higher than normal) levels of tau or tau polypeptides and/or pathological forms of tau. For example, in some cases, a tauopathy is characterized by the presence in brain tissue and/or cerebrospinal fluid of elevated levels of tau or tau polypeptides and/or pathological forms of tau. A “higher than normal” level of tau in a cell, a tissue, or a fluid indicates that the level of tau in the tissue or fluid is higher than a normal, control level, e.g., higher than a normal, control level for an individual or population of individuals of the same age group. In other cases, a tauopathy is characterized by the presence in a cell, a tissue, or a fluid of lower than normal levels of tau. A “lower than normal” level of tau in a tissue or a fluid indicates that the level of tau in the cell, tissue, or fluid is lower than a normal, control level, e.g., lower than a normal, control level for an individual or population of individuals of the same age group. In some cases, an individual having a tauopathy exhibits one or more additional symptoms of a tauopathy (e.g., cognitive decline).

Examples of tauopathies include, but are not limited to, progressive supranuclear palsy (PSP), cerebral amyloid angiopathy, corticobasal degeneration, Creutzfeldt-Jakob disease, dementia pugilistica, diffuse neurofibrillary tangles with calcification, Down's syndrome, frontotemporal dementia (FTD), frontotemporal dementia with parkinsonism linked to chromosome 17, frontotemporal lobar degeneration (FTLD-TAU), corticobasal degeneration, Pick's disease, argyrophilic grain disease, post-encephalitic parkinsonism, chronic traumatic encephalopathy, primary age-related tauopathy, stroke, traumatic brain injury, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, postencephalitic parkinsonism, prion protein cerebral amyloid angiopathy, progressive subcortical gliosis, subacute sclerosing panencephalitis, Tangle only dementia, multi-infarct dementia, ischemic stroke, and Alzheimer's disease (e.g., Irwin D J. Tauopathies as clinicopathological entities. Parkinsonism Relat Disord. 2016; 22 Suppl 1(0 1):S29-S33. doi:10.1016/j.parkreldis.2015.09.020).

The term “progressive supranuclear palsy” or “PSP” refers to a neurologic disorder of unknown origin that gradually destroys cells in many areas of the brain and the accumulation of abnormal aggregates of the microtubule-associated protein tau, resulting in insoluble paired helical filaments, including the gradual deterioration of neurons and glial cells in the midbrain and frontal cortex that display insoluble helical filaments of tau proteins.

PSP starts with a pre-symptomatic phase during which there is an increase in neuropathological abnormalities. Next, patients develop isolated symptoms that are suggestive of PSP (soPSP). In any of the methods described herein, PSP can be classic PSP-Richardson's syndrome (PSP—RS), PSP-Parkinsonism (PSP—P), PSP-corticobasal syndrome (PSP—CBS), PSP-progressive non-fluent aphasia (PSP-PNFA), or PSP-pure akinesia with gait freezing (PSP-PAGF) (Ling et al., J. Mov. Discord. 9(1):3-13, 2016). In some embodiments of any of the methods described herein, a subject can be previously diagnosed or identified as having PSP (e.g., PSP—RS, PSP—P, PSP-PNFA, or PSP-PAGF). In some embodiments of any of the methods described herein, a subject can previously be identified as having an increased risk of developing PSP (e.g., a subject having a genetically related family member (e.g., a parent, grandparent, aunt, uncle, or sibling) that has been identified or diagnosed as having PSP). In some embodiments of any of the methods described herein, a subject can previously be identified or diagnosed as having pre-symptomatic PSP or suggestive-of-PSP.

After onset, symptoms of PSP become rapidly and progressively worse. Subjects diagnosed with PSP may become severely disabled within five years and die within six years.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art.

### Diagnosis and Subject Selection

### Progressive Supranuclear Palsy

In one aspect, the present disclosure provides methods of treating at least one symptom of PSP in a human subject. In some aspects, the present disclosure provides methods of treating a human subject having progressive supranuclear palsy (PSP). Also provided herein are methods of slowing PSP disease progression (e.g., reducing the PSP disease progression rate); and methods of reducing the progressive decline of cognitive functions, including loss of declarative and procedural memory, decreased learning ability, reduced attention span, and severe impairment in thinking ability, judgment, and decision making. Also provided are methods of increasing survival time of a human subject having one or more symptoms of PSP. Also provided are methods of ameliorating one or more biomarkers that is affected in a human subject with PSP (for example, lowering the levels of total tau and phospho-tau, both may be elevated in PSP or lowering the levels of YKL-40, which may also be elevated in PSP). Any of the methods described herein can include administering to the subject a bile acid or a pharmaceutically acceptable salt thereof (e.g., any of the bile acid or a pharmaceutically acceptable salt thereof described herein or known in the art) and a phenylbutyrate compound (e.g., any of the phenylbutyrate compound described herein or known in the art).

Any of the human subjects in the methods described herein may exhibit one or more symptoms associated with PSP or have been diagnosed with PSP. In some embodiments, the subjects may be suspected as having PSP, and/or at risk for developing PSP.

Some embodiments of any of the methods described herein can further include determining that a human subject has or is at risk for developing PSP, diagnosing a human subject as having or at risk for developing PSP, or selecting a human subject having or at risk for developing PSP.

In some embodiments of any of the methods described herein, the human subject has shown one or more symptoms of PSP for about 24 months or less (e.g., about 23, 22, 21, 20, 19, 18, 17, 16, 15, 14, 13, 12, 11, 10, 9, 8, 7, 6, 5, 4, 3, 2, 1 month, or 1 week or less). In some embodiments, the subject has shown one or more symptoms of PSP for about 36 months or less (e.g., about 35, 34, 33, 32, 31, 30, 29, 28, 27, 26, or 25 months or less).

In some instances, the human subject has been diagnosed with PSP. For example, the subject may have been diagnosed with PSP for about 24 months or less (e.g., about 23, 22, 21, 20, 19, 18, 17, 16, 15, 14, 13, 12, 11, 10, 9, 8, 7, 6, 5, 4, 3, 2, or 1 month or less). For example, the subject may have been diagnosed with PSP for 1 week or less, or on the same day that the presently disclosed treatments are administered. The subject may have been diagnosed with PSP for longer than about 24 months (e.g., longer than about 28, 32, 36, 40, 44, 48, 52, 56, 60, 64, 68, 72, 76, or 80 months). Methods of diagnosing PSP are known in the art. For example, the subject can be diagnosed based on clinical history, family history, physical or neurological examinations. The subject can be confirmed or identified, e.g., by a healthcare professional, as having PSP. Multiple parties may be included in the process of diagnosis. For example, where samples are obtained from a subject as part of a diagnosis, a first party can obtain a sample from a subject and a second party can test the sample. In some embodiments of any of the human subjects described herein, the subject is diagnosed, selected, or referred by a medical practitioner (e.g., a general practitioner).

Generally, diagnosis of PSP is known in the art. Symptoms of PSP usually first appear at the age of 60 and worsen until death. People with PSP commonly die from pneumonia, choking or other complications caused by the loss of functional brain cells, resulting in loss of autonomic and motor function (e.g., the ability to swallow).

Signs and symptoms of PSP include movement, cognitive and psychiatric disorders. Voluntary movement can be impaired in PSP and include pseudobulbar palsy (i.e., inability to control facial movements), bradykinesia (i.e., slow or abnormal muscle movement), neck and trunk rigidity, impaired gait, impaired balance, posture instability and difficulty with speech and swallowing. Individuals who become unable to swallow food can be fitted with a feeding tube to provide nutrition. A most obvious, outward sign of the disease is an inability to coordinate and move the eyes normally, resulting in a vertical gaze palsy. Cognitive impairments include loss of executive functions (e.g., attention control, inhibitory control, working memory, cognitive flexibility, reasoning, problem solving and planning) and diminished fluency. Associated psychiatric symptoms include depression, feelings of irritability, sadness or apathy, insomnia, fatigue and loss of energy.

Progressive supranuclear palsy can be difficult to diagnose because signs and symptoms are similar to those of Parkinson's disease. Those of skill in the art may distinguish PSP from Parkinson's based on the lack of tremors, a lot of unexplained falls, little to no response to Parkinson's medications, and/or difficulty moving eyes, particularly downward.

### MDS PSP Diagnostic Criteria

In some embodiments, a subject is identified as having PSP using the MDS PSP Diagnostic Criteria (as described in, e.g., Hoglinger et al., Mov. Disord. 31:644-652, 2016). The diagnostic criteria addresses four functional domains (ocular motor dysfunction, postural instability, akinesia, and cognitive dysfunction) as clinical predictors of PSP.

Progressive Supranuclear Palsy Rating Scale Some embodiments of any of the methods described herein can include monitoring the progression of PSP in the subject, e.g., by assessing the severity of PSP in the subject over time, e.g., using the Progressive Supranuclear Palsy Rating Scale (PSPRS) (e.g., as described in Golbe et al., Brain 130(6):1552-1565, 2007). The PSPRS evaluates subjects according to their ability to perform daily activities, behavior, bulbar function, ocular motor function, limb motor function, and gait. The full PSPRS includes 28 items in six areas. The available total score ranges from 0 (normal) to 100 (maximally disabled). Six items are rated on a 3-point scale (0 to 2) and 22 items are rated on a 5-point scale (0 to 4). The History/Daily Activities area includes seven items with a total maximum of 24 points, the Mentation area four items with 16 points, the Bulbar area two items with 8 points, the Ocular Motor area four items with 16 points, the Limb Motor area six items with 16 points, and the Gait area five items with 20 points.

In addition, a modified version of the PSPRS (10-item PSPRS) that includes only Items 3, 4, 5, 12, 13, 24, 25, 26, 27, 28 from the 28-item PSPRS scale with a modified scoring algorithm will be assessed in this study (provided in the study-specific rating manuals and any supplemental materials).

When both the 28-item and 10-item PSPRS assessments are scheduled, the 28-item PSPRS should be obtained prior to the 10-item PSPRS. At Screening, only the 28-item PSPRS will be performed.

### Movement Disorder Society-Unified Parkinson's Disease Rating Scale Part II

The MDS-UPDRS is made up of 5 parts; however, only Part II will be administered in this study.

The MDS-UPDRS Part II includes 13 items assessing motor aspects of experiences of daily living (Goetz 2007). These items include speech, saliva, and drooling, chewing and swallowing, handwriting, doing hobbies and other activities, eating tasks, tremor, dressing, hygiene, turning in bed, getting out of bed, walking and balance, and freezing.

### Schwab and England Activities of Daily Living Scale

The SEADL scale is a means of assessing a person's ability to perform daily activities in terms of speed and independence through a percentage figure (Schwab 1969). The SEADL is composed of two sections.

The first section is a self-report questionnaire in which participants grade their own daily life activities, such as dressing, using the toilet, resting, eating, and social activities (subjective assessment). The rating will be determined by a qualified staff member according to the participant's self-reported functional ability of specific criteria, with 100% indicating total independence and falling to 0% indicating a state of complete dependence.

The second section is an assessment of motor functions, such as postural balance, speaking, rigidity, and tremors, and will be conducted by a clinician (objective assessment).

### EuroQuality of Life

The EQ5D-5L contains a health state descriptive part comprising five items, scored from 1 (no problems or symptoms) to 3 (serious problems or symptoms), a question about change in health state in the preceding 12 months, and a visual analog scale (VAS) to evaluate current health state (from 0, worst imaginable, to 100, best imaginable). The descriptive profile can be converted into a value (EQ-Index) which ranges from 0 (death) to 1 (perfect health), with negative values indicating health states considered worse than death.

### Zarit Burden Interview

The Zarit Scale of Caregiver Burden or the Zarit Burden Interview is used to assess caregiver burden. The short version contains 22 items. Each item of the interview is a statement which the consented study partner of the participant is asked to endorse using a 5-point scale. Response options range from 0 (Never) to 4 (Nearly Always).

### Clinical Global Impression of Severity and Change

The CGI-S is a clinician's rating of disease severity. The CGI-S rates severity of illness on a 7-point scale, using a range of responses from 1 (normal) through to 7 (the most severely ill). This rating is based upon observed and reported symptoms, behavior, and function in the past 7 days.

The CGI-C rates improvement by 7 categories: very much improved, much improved, minimally improved, no change, minimally worse, much worse, very much worse.

The CGI-C will be assessed relative to the CGI-S at the Week 1 (Day 1) visit as the reference.

### Mini-Mental State Exam

The MMSE is a brief, 30-point questionnaire that provides a quantitative measure of cognitive status in adults and is widely used to screen for cognitive impairment and to estimate the severity of cognitive impairment at a given point in time. The MMSE total score ranges from 0 to 30, with lower score indicating greater impairment (Folstein 1975).

In some instances, the MMSE is used for determining if the subject should receive TURSO and sodium phenylbutyrate, as described herein. The participant must have a score of ≥24.

### Montreal Cognitive Assessment

The MoCA was designed as a rapid screening instrument for mild cognitive dysfunction (Nasreddine 2005). It assesses different cognitive domains: attention and concentration, executive functions, memory, language, visuoconstructional skills, conceptual thinking, calculations, and orientation.

### Columbia-Suicide Severity Rating Scale

The C—SSRS is a systematically administered instrument developed to track suicidal AEs across a treatment study. The instrument is designed to assess suicidal behavior and ideation, track and assess all suicidal events, as well as the lethality of attempts. Additional features assessed include frequency, duration, controllability, reason for ideation, and deterrents. The C—SSRS is considered a low-burden instrument as it takes less than 5 minutes to administer.

### Genetic Alterations

In some embodiments, a subject is identified as being at increased risk of developing PSP or identified as having PSP (e.g., any of the types of PSP described herein), e.g., at least in part, by detecting a genetic alteration in a gene encoding the microtubule-associated protein tau (MAPT) (e.g., any of the inversion polymorphisms in the MAPT gene, any of the haplotype-specific polymorphisms in the MAPT gene, the rare-coding MAPT variant (A152T), or mutations that enhance splicing of exon 10 in the MAPT gene described, e.g., in Hoglinger et al., Nature Genet. 43:699-705, 2011, and Hinz et al., Cold Spring Harb. Perspect Biol.). Non-limiting examples of genetic alterations in a gene encoding MAPT include mutations that result in the production of MAPT protein that include one or more point mutations of: S285R, L284R, P301L, and G303V. Additional specific genetic mutations in a gene encoding MAPT protein that can be used to identify a subject as having an increased risk of developing PSP or can be used to identify a subject as having PSP (e.g., any of the types of PSP described herein) are described in, e.g., Boxer et al., Lancet 16:552-563, 2017.

### Scanning and Neuroimaging

In some embodiments, a subject is identified as having an increased risk of developing PSP or identified as having PSP (e.g., any of the types of PSP described herein), e.g., at least in part, by detecting tau protein deposits (e.g., 4-repeat tau protein deposits), detecting of atrophy of the midbrain and/or superior cerebellar peduncles (e.g., using any of the imaging techniques described herein or known in the art, e.g., magnetic resonance imaging (MRI) or positron emission tomography (PET) scans), and/or detecting of hypometabolism in the frontal cortex, caudate, and/or thalamus in the subject (e.g., using any of the imaging techniques described herein or known in the art, e.g., MRI, CT scan, or PET scan).

For example, in some embodiments a subject is identified or diagnosed as having PSP (e.g., any of the types of PSP described herein), e.g., at least in part, by using MRI to detect brain atrophy (Min et al., Nat. Med. 21:1154-1162, 2015; Yanamandra et al., Ann. Clin. Transl. Neurol. 2:278-288, 2015), changes in regional gray and white matter volume to detect atrophy (see, e.g., Josephs et al., Brain 137:2783-2795, 2014; Santos-Santos et al., JAMA Neurol. 73:733-742, 2016), and midbrain atrophy by detecting midbrain area and volume in the subject (Josephs et al., Neurobiol. Aging 29:280-289, 2008; Whitwell et al., Eur. J. Neurol. 20:1417-1422, 2013). In some embodiments, a subject can be identified or diagnosed as having PSP (e.g., any of the types of PSP described herein), e.g., at least in part, by administering to a subject a tau protein tracer (e.g., AV1451 or PBB3) and detecting tau protein in the subject's brain using a PET scan (see, e.g., Marquie et al., Ann. Neurol. 78:787-800, 2015; Cho et al., Mov. Disord. 32:134-140, 2017; Whitwell et al., Mov. Disord. 32:124-133, 2017; and Smith et al., Mov. Disord. 32:108-114, 2017). In some embodiments, a subject can be diagnosed or identified as having PSP (e.g., any of the types of PSP described herein), e.g., at least in part, by detecting the difference in binaural masking level in the subject using a PET scan (see, e.g., Hughes et al., J. Neurophysiol. 112:3086-3094, 2014).

### Blood (e.g., Plasma) or CSFPSP Biomarkers

In some embodiments, a subject is identified as being at increased risk of developing PSP or identified as having PSP (e.g., any of the types of PSP described herein), e.g., at least in part, by detecting the presence of, or an elevated level (e.g., as compared to a level in a healthy control subject) of, one or more biomarkers in a subject.

### Tau and Phospho-Tau

Subjects in the methods described herein may have a plasma or a CSF total tau level that is elevated as compared to a healthy subject. For example, the subject may have plasma or CSF total tau level of about 100 pg/mL or higher. In some embodiments, the subjects have plasma or CSF total tau level of about 300 pg/mL or higher (e.g., about 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 3000, 3200, 3500, 3800, or 4000 pg/mL or higher).

Subjects in the methods described herein may have plasma or CSF phospho-tau (e.g., phospho-tau 181, phospho-tau 199, and/or phosphor-tau 231) that is elevated as compared to a healthy subject. For example, the subject may have plasma or CSF phospho-tau level of about pg/mL or higher. In some embodiments, the subjects have plasma or CSF phospho-Tau (e.g., phosphor-tau 181) level about 70 pg/mL or higher (e.g., about 75, 100, 125, 150, 175, or 200 pg/mL or higher).

### Neurofilament-Light (NfL)

In some embodiments, a subject is identified or diagnosed as having PSP (e.g., any of the types of PSP described herein), e.g., at least in part, by detecting the presence of, or detection of an elevated level (e.g., as compared to a level in a healthy control subject) of, neurofilament light chain in the blood and/or cerebrospinal fluid in a subject (e.g., using any of the immunoassays described in Scherling et al., Ann. Neurol. 75:116-126, 2014; Bacioglu et al., Neuron 91:56-66, 2016; and Rojas et al., Ann. Clin. Transl. Neurol. 3:216-255, 2016).

Methods of detecting NfL (for example, in the cerebrospinal fluid, plasma, or serum) are known in the art and include but are not limited to, ELISA and Simoa assays (See e.g., Shaw et al. Biochemical and Biophysical Research Communications 336:1268-1277, 2005; Ganesalingam et al. Amyotroph Lateral Scler Frontotemporal Degener 14(2):146-9, 2013; De Schaepdryver et al. Annals of Clinical and Translational Neurology 6(10): 1971-1979, 2019; Wilke et al. Clin Chem Lab Med 57(10):1556-1564, 2019; Poesen et al. Front Neurol 9:1167, 2018; Pawlitzki et al. Front. Neurol. 9:1037, 2018; Gille et al. Neuropathol Appl Neurobiol 45(3):291-304, 2019). Commercial NfL assay kits based on the Simoa technology, such as those produced by Quanterix can also be used (See, e.g., Thouvenot et al. European Journal of Neurology 27:251-257, 2020). Factors affecting NfL levels or their detection in serum or plasma in relation to disease course may differ from those in CSF. The levels of neurofilament (e.g., pNF-H and/or NfL) in the CSF and serum may be correlated (See, e.g., Wilke et al. Clin Chem Lab Med 57(10):1556-1564, 2019).

### YKL-40

In some embodiments, a subject is identified or diagnosed as having PSP (e.g., any of the types of PSP described herein), e.g., at least in part, by detecting the presence of, or detection of an elevated level (e.g., as compared to a level in a healthy control subject) of, YKL-40 in plasma or cerebrospinal fluid from the subject (see, e.g., Magdalinou et al., J Neurol. Neurosurg. Psychiatry 2014 October; 85(10): 1065-1075; and Magdalinou et al., J Neurol. Neurosurg. Psychiatry 86:1240-1247, 2015).

### Ratio of 33 kDa Tau to 55 kDa Tau

In some embodiments, a subject is identified or diagnosed as having PSP (e.g., any of the types of PSP described herein), e.g., at least in part, by detecting a decreased ratio of 33 kDa tau to 55 kDa tau in the plasma or CSF of a subject (e.g., as compared to the ratio of 33 kDa tau to 55 kDa tau in a healthy subject).

### Ptpn1

In some embodiments, a subject is identified or diagnosed as having PSP (e.g., any of the types of PSP described herein), e.g., at least in part, by detecting the presence of, or detecting an elevated level of protein tyrosine phosphatase 1 (Ptpn1) (e.g., as described in Santiago et al., Mov. Discord. 29(4):550-555, 2014).

### Neurogranin

In some embodiments, a subject is identified or diagnosed as having PSP (e.g., any of the types of PSP described herein), e.g., at least in part, by detecting the presence of, or detecting an elevated level of neurogranin (see, e.g., Xiang Y, Xin J, Le W, Yang Y. Neurogranin: A Potential Biomarker of Neurological and Mental Diseases. Front Aging Neurosci. 2020-10-6; 12:584743. doi: 10.3389/fnagi.2020.584743.).

### Other Factors Useful for Diagnosis/Subject Selection for PSP

In some embodiments, a subject is identified or diagnosed as having PSP (e.g., any of the types of PSP described herein), e.g., at least in part, by detecting decreased saccade velocity and gain in the subject using infrared oculography (see, e.g., Boxer et al., Arch. Neurol. 69:509-517, 2012; Boxer et al., Lancet Neurol. 132:676-685, 2014). In some embodiments, a subject is identified or diagnosed as having PSP (e.g., any of the types of PSP described herein), e.g., at least in part, by detecting a spontaneous and evoked blink rate associated with PSP in the subject (see, e.g., Bologna et al., Brain 132:502-510, 2009). In some embodiments, a subject is identified or diagnosed as having PSP (e.g., any of the types of PSP described herein), e.g., at least in part, by detecting decreased retinal thickness in a subject's eye using optical coherence tomography (see, e.g., Schneider et al., J. Neural Transm. 121:41-47, 2014). In some embodiments, a subject is identified or diagnosed as having PSP (e.g., any of the types of PSP described herein), e.g., at least in part, by detecting disrupted circadian rhythms and sleep in the subject (see, e.g., Walsh et al., Sleep Med. 22; 50-56, 2016).

General Selection of Subjects with Various Neurodegenerative Diseases

In some embodiments, a subject is selected based on the presence of one or more of the following criteria:
Is an adult between the age of 40-80 years old
Possible or Probable PSP (Steele-Richardson-Olszewski Syndrome) according to MDS 2017 criteria (Gradually progressive disorder, with age at disease onset ≥40 years 2. Either or both of the following two items are met: i. Vertical supranuclear gaze palsy OR slow velocity of vertical saccades AND postural instability with repeated unprovoked falls within 3 years OR tendency to fall on the pull-test within 3 years ii. Slow velocity of vertical saccades AND postural instability with more than two steps backward on the pull-test within 3 years.)
Presence of PSP symptoms <5 years
Able to walk independently (Ability to walk 5 steps with minimal assistance (stabilization of one arm)
PSPRS total score <40
MMSE score ≥24
No feeding tube use.

In some embodiments, the subjects described herein have an “elevated level” of a biomarker (e.g., tau, phospho-tau, NfL, YKL-40, Ptpn1, or neurogranin) in the CSF or blood as compared to a healthy subject who does not have PSP. In some embodiments, an elevated level of a PSP subject can be an elevation or an increase of about 1% to about 500%, about 1% to about 450%, about 1% to about 400%, about 1% to about 350%, about 1% to about 300%, about 1% to about 250%, about 1% to about 200%, about 1% to about 150%, about 1% to about 100%, about 1% to about 50%, about 1% to about 25%, about 1% to about 20%, about 1% to about 15%, about 1% to about 10%, about 1% to about 5%, about 2% to about 500%, about 2% to about 450%, about 2% to about 400%, about 2% to about 350%, about 2% to about 300%, about 2% to about 250%, about 2% to about 200%, about 2% to about 150%, about 2% to about 100%, about 2% to about 50%, about 2% to about 25%, about 2% to about 20%, about 2% to about 15%, about 2% to about 10%, about 5% to about 500%, about 5% to about 450%, about 5% to about 400%, about 5% to about 350%, about 5% to about 300%, about 5% to about 250%, about 5% to about 200%, about 5% to about 150%, about 5% to about 100%, about 5% to about 50%, about 5% to about 25%, about 5% to about 20%, about 5% to about 15%, about 5% to about 10%, about 10% to about 500%, about 10% to about 450%, about 10% to about 400%, about 10% to about 350%, about 10% to about 300%, about 10% to about 250%, about 10% to about 200%, about 10% to about 150%, about 10% to about 100%, about 10% to about 50%, about 10% to about 25%, about 10% to about 20%, about 10% to about 15%, about 15% to about 500%, about 15% to about 450%, about 15% to about 400%, about 15% to about 350%, about 15% to about 300%, about 15% to about 250%, about 15% to about 200%, about 15% to about 150%, about 15% to about 100%, about 15% to about 50%, about 15% to about 25%, about 15% to about 20%, about 20% to about 500%, about 20% to about 450%, about 20% to about 400%, about 20% to about 350%, about 20% to about 300%, about 20% to about 250%, about 20% to about 200%, about 20% to about 150%, about 20% to about 100%, about 20% to about 50%, about 20% to about 25%, about 25% to about 500%, about 25% to about 450%, about 25% to about 400%, about 25% to about 350%, about 25% to about 300%, about 25% to about 250%, about 25% to about 200%, about 25% to about 150%, about 25% to about 100%, about 25% to about 50%, about 50% to about 500%, about 50% to about 450%, about 50% to about 400%, about 50% to about 350%, about 50% to about 300%, about 50% to about 250%, about 50% to about 200%, about 50% to about 150%, about 50% to about 100%, about 100% to about 500%, about 100% to about 450%, about 100% to about 400%, about 100% to about 350%, about 100% to about 300%, about 100% to about 250%, about 100% to about 200%, about 100% to about 150%, about 150% to about 500%, about 150% to about 450%, about 150% to about 400%, about 150% to about 350%, about 150% to about 300%, about 150% to about 250%, about 150% to about 200%, about 200% to about 500%, about 200% to about 450%, about 200% to about 400%, about 200% to about 350%, about 200% to about 300%, about 200% to about 250%, about 250% to about 500%, about 250% to about 450%, about 250% to about 400%, about 250% to about 350%, about 250% to about 300%, about 300% to about 500%, about 300% to about 450%, about 300% to about 400%, about 300% to about 350%, about 350% to about 500%, about 350% to about 450%, about 350% to about 400%, about 400% to about 500%, about 400% to about 450%, or about 450% to about 500%, e.g., as compared to a healthy subject who does not have PSP.

In some embodiments, after administration with any of the compositions described herein (e.g., TURSO and sodium phenylbutyrate), the subjects have a reduction in the level (plasma or CSF) of a biomarker (e.g., tau, phospho-tau, NfL, YKL-40, or Ptpn1). For example, a 1% to about 99% reduction, a 1% to about 95% reduction, a 1% to about 90% reduction, a 1% to about 85% reduction, a 1% to about 80% reduction, a 1% to about 75% reduction, a 1% to about 70% reduction, a 1% to about 65% reduction, a 1% to about 60% reduction, a 1% to about 55% reduction, a 1% to about 50% reduction, a 1% to about 45% reduction, a 1% to about 40% reduction, a 1% to about 35% reduction, a 1% to about 30% reduction, a 1% to about 25% reduction, a 1% to about 20% reduction, a 1% to about 15% reduction, a 1% to about 10% reduction, a 1% to about 5% reduction, an about 5% to about 99% reduction, an about 5% to about 95% reduction, an about 5% to about 90% reduction, an about 5% to about 85% reduction, an about 5% to about 80% reduction, an about 5% to about 75% reduction, an about 5% to about 70% reduction, an about 5% to about 65% reduction, an about 5% to about 60% reduction, an about 5% to about 55% reduction, an about 5% to about 50% reduction, an about 5% to about 45% reduction, an about 5% to about 40% reduction, an about 5% to about 35% reduction, an about 5% to about 30% reduction, an about 5% to about 25% reduction, an about 5% to about 20% reduction, an about 5% to about 15% reduction, an about 5% to about 10% reduction, an about 10% to about 99% reduction, an about 10% to about 95% reduction, an about 10% to about 90% reduction, an about 10% to about 85% reduction, an about 10% to about 80% reduction, an about 10% to about 75% reduction, an about 10% to about 70% reduction, an about 10% to about 65% reduction, an about 10% to about 60% reduction, an about 10% to about 55% reduction, an about 10% to about 50% reduction, an about 10% to about 45% reduction, an about 10% to about 40% reduction, an about 10% to about 35% reduction, an about 10% to about 30% reduction, an about 10% to about 25% reduction, an about 10% to about 20% reduction, an about 10% to about 15% reduction, an about 15% to about 99% reduction, an about 15% to about 95% reduction, an about 15% to about 90% reduction, an about 15% to about 85% reduction, an about 15% to about 80% reduction, an about 15% to about 75% reduction, an about 15% to about 70% reduction, an about 15% to about 65% reduction, an about 15% to about 60% reduction, an about 15% to about 55% reduction, an about 15% to about 50% reduction, an about 15% to about 45% reduction, an about 15% to about 40% reduction, an about 15% to about 35% reduction, an about 15% to about 30% reduction, an about 15% to about 25% reduction, an about 15% to about 20% reduction, an about 20% to about 99% reduction, an about 20% to about 95% reduction, an about 20% to about 90% reduction, an about 20% to about 85% reduction, an about 20% to about 80% reduction, an about 20% to about 75% reduction, an about 20% to about 70% reduction, an about 20% to about 65% reduction, an about 20% to about 60% reduction, an about 20% to about 55% reduction, an about 20% to about 50% reduction, an about 20% to about 45% reduction, an about 20% to about 40% reduction, an about 20% to about 35% reduction, an about 20% to about 30% reduction, an about 20% to about 25% reduction, an about 25% to about 99% reduction, an about 25% to about 95% reduction, an about 25% to about 90% reduction, an about 25% to about 85% reduction, an about 25% to about 80% reduction, an about 25% to about 75% reduction, an about 25% to about 70% reduction, an about 25% to about 65% reduction, an about 25% to about 60% reduction, an about 25% to about 55% reduction, an about 25% to about 50% reduction, an about 25% to about 45% reduction, an about 25% to about 40% reduction, an about 25% to about 35% reduction, an about 25% to about 30% reduction, an about 30% to about 99% reduction, an about 30% to about 95% reduction, an about 30% to about 90% reduction, an about 30% to about 85% reduction, an about 30% to about 80% reduction, an about 30% to about 75% reduction, an about 30% to about 70% reduction, an about 30% to about 65% reduction, an about 30% to about 60% reduction, an about 30% to about 55% reduction, an about 30% to about 50% reduction, an about 30% to about 45% reduction, an about 30% to about 40% reduction, an about 30% to about 35% reduction, an about 35% to about 99% reduction, an about 35% to about 95% reduction, an about 35% to about 90% reduction, an about 35% to about 85% reduction, an about 35% to about 80% reduction, an about 35% to about 75% reduction, an about 35% to about 70% reduction, an about 35% to about 65% reduction, an about 35% to about 60% reduction, an about 35% to about 55% reduction, an about 35% to about 50% reduction, an about 35% to about 45% reduction, an about 35% to about 40% reduction, an about 40% to about 99% reduction, an about 40% to about 95% reduction, an about 40% to about 90% reduction, an about 40% to about 85% reduction, an about 40% to about 80% reduction, an about 40% to about 75% reduction, an about 40% to about 70% reduction, an about 40% to about 65% reduction, an about 40% to about 60% reduction, an about 40% to about 55% reduction, an about 40% to about 50% reduction, an about 40% to about 45% reduction, an about 45% to about 99% reduction, an about 45% to about 95% reduction, an about 45% to about 90% reduction, an about 45% to about 85% reduction, an about 45% to about 80% reduction, an about 45% to about 75% reduction, an about 45% to about 70% reduction, an about 45% to about 65% reduction, an about 45% to about 60% reduction, an about 45% to about 55% reduction, an about 45% to about 50% reduction, an about 50% to about 99% reduction, an about 50% to about 95% reduction, an about 50% to about 90% reduction, an about 50% to about 85% reduction, an about 50% to about 80% reduction, an about 50% to about 75% reduction, an about 50% to about 70% reduction, an about 50% to about 65% reduction, an about 50% to about 60% reduction, an about 50% to about 55% reduction, an about 55% to about 99% reduction, an about 55% to about 95% reduction, an about 55% to about 90% reduction, an about 55% to about 85% reduction, an about 55% to about 80% reduction, an about 55% to about 75% reduction, an about 55% to about 70% reduction, an about 55% to about 65% reduction, an about 55% to about 60% reduction, an about 60% to about 99% reduction, an about 60% to about 95% reduction, an about 60% to about 90% reduction, an about 60% to about 85% reduction, an about 60% to about 80% reduction, an about 60% to about 75% reduction, an about 60% to about 70% reduction, an about 60% to about 65% reduction, an about 65% to about 99% reduction, an about 65% to about 95% reduction, an about 65% to about 90% reduction, an about 65% to about 85% reduction, an about 65% to about 80% reduction, an about 65% to about 75% reduction, an about 65% to about 70% reduction, an about 70% to about 99% reduction, an about 70% to about 95% reduction, an about 70% to about 90% reduction, an about 70% to about 85% reduction, an about 70% to about 80% reduction, an about 70% to about 75% reduction, an about 75% to about 99% reduction, an about 75% to about 95% reduction, an about 75% to about 90% reduction, an about 75% to about 85% reduction, an about 75% to about 80% reduction, an about 80% to about 99% reduction, an about 80% to about 95% reduction, an about 80% to about 90% reduction, an about 80% to about 85% reduction, an about 85% to about 99% reduction, an about 85% to about 95% reduction, an about 85% to about 90% reduction, an about 90% to about 99% reduction, an about 90% to about 95% reduction, or an about 95% to about 99% reduction, e.g., in a second level (i.e., after treatment with the compositions described herein) of a biomarker (e.g., tau, phospho-tau, NfL, YKL-40, or Ptpn1) as compared to a first level (i.e., prior to treatment with the compositions described herein) of the biomarker (e.g., tau, phospho-tau, NfL, YKL-40, or Ptpn1).

### Bioavailability/Metabolism

Skilled practitioners will appreciate that certain factors can affect the bioavailability and metabolism of the administered compounds for a subject, and can make adjustments accordingly. These include but are not limited to liver function (e.g., levels of liver enzymes), renal function, and gallbladder function (e.g., ion absorption and secretion, levels of cholesterol transport proteins). There can be variability in the levels of exposure each subject has for the administered compounds (e.g., bile acid and a phenylbutyrate compound), differences in the levels of excretion, and in the pharmacokinetics of the compounds in the subjects being treated. Any of the factors described herein may affect drug exposure by the subject. For instance, decreased clearance of the compounds can result in increased drug exposure, while improved renal function can reduce the actual drug exposure. The extent of drug exposure may be correlated with the subject's response to the administered compounds and the outcome of the treatment.

### Subject Age

The subject can be e.g., older than 18 years of age (e.g., between 18-100, 18-90, 18-80, 18-70, 18-60, 18-50, 18-40, 18-30, 18-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 years of age). The subject can have a BMI of between 18.5-30 kg/m² (e.g., between 18.5-28, 18.5-26, 18.5-24, 18.5-22, 18.5-20, 20-30, 20-28, 20-26, 20-24, 20-22, 22-30, 22-28, 22-26, 22-24, 24-30, 24-28, 24-26, 26-30, 26-28, or 28-30 kg/m²).

### Methods of Treatment

The present disclosure provides methods of treating a neurodegenerative disease (e.g., PSP) in a subject, or ameliorating at least one symptom of a neurodegenerative disease (e.g., PSP) in a subject, or prophylactically treating a subject at risk for developing a neurodegenerative disease (e.g., PSP) (e.g., a subject who carries one or more copies of the ApoEε4 allele) or a subject suspected to be developing a neurodegenerative disease (e.g., a subject displaying at least one symptom of PSP).

Some embodiments of the present disclosure provide methods of slowing a neurodegenerative disease (e.g., PSP) disease progression (e.g., reducing the PSP disease progression rate); and methods of reducing and/or preventing progressive decline of cognitive functions, including loss of declarative and procedural memory, decreased learning ability, reduced attention span, and severe impairment in thinking ability, judgment, and decision making.

Also provided herein are methods of ameliorating one or more biomarkers (e.g., those that are associated with neuronal injury and neuro-inflammation) that are affected in a neurodegenerative disease (e.g., PSP). For example, in some instances, provided herein are methods to reduce total tau and/or phospho-tau in the CSF, serum, or blood, etc.

Generally, also provided in the present disclosure are methods of treating a tauopathy in a subject, or ameliorating at least one symptom of a tauopathy in a subject, or prophylactically treating a subject at risk for developing a tauopathy or a subject suspected to be developing a tauopathy. Some embodiments of the present disclosure provide methods of slowing a tauopathy disease progression; and methods of reducing and/or preventing progressive decline of various functions associated with the tauopathy (e.g., in some instances this may be cognitive functions). Also provided herein are methods of ameliorating one or more biomarkers that are affected in a tauopathy patients.

In some embodiments of any of the methods described herein, the methods include administering to the subject a bile acid or pharmaceutically acceptable salt thereof, and a phenylbutyrate compound. In some embodiments, the methods described herein include administering to a subject about 10 mg/kg to about 50 mg/kg (e.g., about 10 mg/kg to about 48 mg/kg, about 10 mg/kg to about 46 mg/kg, about 10 mg/kg to about 44 mg/kg, about 10 mg/kg to about 42 mg/kg, about 10 mg/kg to about 40 mg/kg, about 10 mg/kg to about 38 mg/kg, about 10 mg/kg to about 36 mg/kg, about 10 mg/kg to about 34 mg/kg, about 10 mg/kg to about 32 mg/kg, about 10 mg/kg to about 30 mg/kg, about 10 mg/kg to about 28 mg/kg, about 10 mg/kg to about 26 mg/kg, about 10 mg/kg to about 24 mg/kg, about 10 mg/kg to about 22 mg/kg, about 10 mg/kg to about 20 mg/kg, about 10 mg/kg to about 18 mg/kg, about 10 mg/kg to about 16 mg/kg, about 10 mg/kg to about 14 mg/kg, about 10 mg/kg to about 12 mg/kg, about 12 mg/kg to about 50 mg/kg, about 12 mg/kg to about 48 mg/kg, about 12 mg/kg to about 46 mg/kg, about 12 mg/kg to about 44 mg/kg, about 12 mg/kg to about 42 mg/kg, about 12 mg/kg to about 40 mg/kg, about 12 mg/kg to about 38 mg/kg, about 12 mg/kg to about 36 mg/kg, about 12 mg/kg to about 34 mg/kg, about 12 mg/kg to about 32 mg/kg, about 12 mg/kg to about 30 mg/kg, about 12 mg/kg to about 28 mg/kg, about 12 mg/kg to about 26 mg/kg, about 12 mg/kg to about 24 mg/kg, about 12 mg/kg to about 22 mg/kg, about 12 mg/kg to about 20 mg/kg, about 12 mg/kg to about 18 mg/kg, about 12 mg/kg to about 16 mg/kg, about 12 mg/kg to about 14 mg/kg, about 14 mg/kg to about 50 mg/kg, about 14 mg/kg to about 48 mg/kg, about 14 mg/kg to about 46 mg/kg, about 14 mg/kg to about 44 mg/kg, about 14 mg/kg to about 42 mg/kg, about 14 mg/kg to about 40 mg/kg, about 14 mg/kg to about 38 mg/kg, about 14 mg/kg to about 36 mg/kg, about 14 mg/kg to about 34 mg/kg, about 14 mg/kg to about 32 mg/kg, about 14 mg/kg to about 30 mg/kg, about 14 mg/kg to about 28 mg/kg, about 14 mg/kg to about 26 mg/kg, about 14 mg/kg to about 24 mg/kg, about 14 mg/kg to about 22 mg/kg, about 14 mg/kg to about 20 mg/kg, about 14 mg/kg to about 18 mg/kg, about 14 mg/kg to about 16 mg/kg, about 16 mg/kg to about 50 mg/kg, about 16 mg/kg to about 48 mg/kg, about 16 mg/kg to about 46 mg/kg, about 16 mg/kg to about 44 mg/kg, about 16 mg/kg to about 42 mg/kg, about 16 mg/kg to about 40 mg/kg, about 16 mg/kg to about 38 mg/kg, about 16 mg/kg to about 36 mg/kg, about 16 mg/kg to about 34 mg/kg, about 16 mg/kg to about 32 mg/kg, about 16 mg/kg to about 30 mg/kg, about 16 mg/kg to about 28 mg/kg, about 16 mg/kg to about 26 mg/kg, about 16 mg/kg to about 24 mg/kg, about 16 mg/kg to about 22 mg/kg, about 16 mg/kg to about 20 mg/kg, about 16 mg/kg to about 18 mg/kg, about 18 mg/kg to about 50 mg/kg, about 18 mg/kg to about 48 mg/kg, about 18 mg/kg to about 46 mg/kg, about 18 mg/kg to about 44 mg/kg, about 18 mg/kg to about 42 mg/kg, about 18 mg/kg to about 40 mg/kg, about 18 mg/kg to about 38 mg/kg, about 18 mg/kg to about 36 mg/kg, about 18 mg/kg to about 34 mg/kg, about 18 mg/kg to about 32 mg/kg, about 18 mg/kg to about 30 mg/kg, about 18 mg/kg to about 28 mg/kg, about 18 mg/kg to about 26 mg/kg, about 18 mg/kg to about 24 mg/kg, about 18 mg/kg to about 22 mg/kg, about 18 mg/kg to about 20 mg/kg, about 20 mg/kg to about 50 mg/kg, about 20 mg/kg to about 48 mg/kg, about 20 mg/kg to about 46 mg/kg, about 20 mg/kg to about 44 mg/kg, about 20 mg/kg to about 42 mg/kg, about 20 mg/kg to about 40 mg/kg, about 20 mg/kg to about 38 mg/kg, about 20 mg/kg to about 36 mg/kg, about 20 mg/kg to about 34 mg/kg, about 20 mg/kg to about 32 mg/kg, about 20 mg/kg to about 30 mg/kg, about 20 mg/kg to about 28 mg/kg, about 20 mg/kg to about 26 mg/kg, about 20 mg/kg to about 24 mg/kg, about 20 mg/kg to about 22 mg/kg, about 22 mg/kg to about 50 mg/kg, about 22 mg/kg to about 48 mg/kg, about 22 mg/kg to about 46 mg/kg, about 22 mg/kg to about 44 mg/kg, about 22 mg/kg to about 42 mg/kg, about 22 mg/kg to about 40 mg/kg, about 22 mg/kg to about 38 mg/kg, about 22 mg/kg to about 36 mg/kg, about 22 mg/kg to about 34 mg/kg, about 22 mg/kg to about 32 mg/kg, about 22 mg/kg to about 30 mg/kg, about 22 mg/kg to about 28 mg/kg, about 22 mg/kg to about 26 mg/kg, about 22 mg/kg to about 24 mg/kg, about 24 mg/kg to about 50 mg/kg, about 24 mg/kg to about 48 mg/kg, about 24 mg/kg to about 46 mg/kg, about 24 mg/kg to about 44 mg/kg, about 24 mg/kg to about 42 mg/kg, about 24 mg/kg to about 40 mg/kg, about 24 mg/kg to about 38 mg/kg, about 24 mg/kg to about 36 mg/kg, about 24 mg/kg to about 34 mg/kg, about 24 mg/kg to about 32 mg/kg, about 24 mg/kg to about 30 mg/kg, about 24 mg/kg to about 28 mg/kg, about 24 mg/kg to about 26 mg/kg, about 26 mg/kg to about 50 mg/kg, about 26 mg/kg to about 48 mg/kg, about 26 mg/kg to about 46 mg/kg, about 26 mg/kg to about 44 mg/kg, about 26 mg/kg to about 42 mg/kg, about 26 mg/kg to about 40 mg/kg, about 26 mg/kg to about 38 mg/kg, about 26 mg/kg to about 36 mg/kg, about 26 mg/kg to about 34 mg/kg, about 26 mg/kg to about 32 mg/kg, about 26 mg/kg to about 30 mg/kg, about 26 mg/kg to about 28 mg/kg, about 28 mg/kg to about 50 mg/kg, about 28 mg/kg to about 48 mg/kg, about 28 mg/kg to about 46 mg/kg, about 28 mg/kg to about 44 mg/kg, about 28 mg/kg to about 42 mg/kg, about 28 mg/kg to about 40 mg/kg, about 28 mg/kg to about 38 mg/kg, about 28 mg/kg to about 36 mg/kg, about 28 mg/kg to about 34 mg/kg, about 28 mg/kg to about 32 mg/kg, about 28 mg/kg to about 30 mg/kg, about 30 mg/kg to about 50 mg/kg, about 30 mg/kg to about 48 mg/kg, about 30 mg/kg to about 46 mg/kg, about 30 mg/kg to about 44 mg/kg, about 30 mg/kg to about 42 mg/kg, about 30 mg/kg to about 40 mg/kg, about 30 mg/kg to about 38 mg/kg, about 30 mg/kg to about 36 mg/kg, about 30 mg/kg to about 34 mg/kg, about 30 mg/kg to about 32 mg/kg, about 32 mg/kg to about 50 mg/kg, about 32 mg/kg to about 48 mg/kg, about 32 mg/kg to about 46 mg/kg, about 32 mg/kg to about 44 mg/kg, about 32 mg/kg to about 42 mg/kg, about 32 mg/kg to about 40 mg/kg, about 32 mg/kg to about 38 mg/kg, about 32 mg/kg to about 36 mg/kg, about 32 mg/kg to about 34 mg/kg, about 34 mg/kg to about 50 mg/kg, about 34 mg/kg to about 48 mg/kg, about 34 mg/kg to about 46 mg/kg, about 34 mg/kg to about 44 mg/kg, about 34 mg/kg to about 42 mg/kg, about 34 mg/kg to about 40 mg/kg, about 34 mg/kg to about 38 mg/kg, about 34 mg/kg to about 36 mg/kg, about 36 mg/kg to about 50 mg/kg, about 36 mg/kg to about 48 mg/kg, about 36 mg/kg to about 46 mg/kg, about 36 mg/kg to about 44 mg/kg, about 36 mg/kg to about 42 mg/kg, about 36 mg/kg to about 40 mg/kg, about 36 mg/kg to about 38 mg/kg, about 38 mg/kg to about 50 mg/kg, about 38 mg/kg to about 48 mg/kg, about 38 mg/kg to about 46 mg/kg, about 38 mg/kg to about 44 mg/kg, about 38 mg/kg to about 42 mg/kg, about 38 mg/kg to about 40 mg/kg, about 40 mg/kg to about 50 mg/kg, about 40 mg/kg to about 48 mg/kg, about 40 mg/kg to about 46 mg/kg, about 40 mg/kg to about 44 mg/kg, about 40 mg/kg to about 42 mg/kg, about 42 mg/kg to about 50 mg/kg, about 42 mg/kg to about 48 mg/kg, about 42 mg/kg to about 46 mg/kg, about 42 mg/kg to about 44 mg/kg, about 44 mg/kg to about 50 mg/kg, about 44 mg/kg to about 48 mg/kg, about 44 mg/kg to about 46 mg/kg, about 46 mg/kg to about 50 mg/kg, about 46 mg/kg to about 48 mg/kg, or about 46 mg/kg to about 50 mg/kg) of body weight of a bile acid (e.g., any of the bile acids described herein or known in the art e.g., TURSO) or a pharmaceutically acceptable salt thereof, and about 10 mg/kg to about 400 mg/kg (e.g., about 10 mg/kg to about 380 mg/kg, about 10 mg/kg to about 360 mg/kg, about 10 mg/kg to about 340 mg/kg, about 10 mg/kg to about 320 mg/kg, about 10 mg/kg to about 300 mg/kg, about 10 mg/kg to about 280 mg/kg, about 10 mg/kg to about 260 mg/kg, about 10 mg/kg to about 240 mg/kg, about 10 mg/kg to about 220 mg/kg, about 10 mg/kg to about 200 mg/kg, about 10 mg/kg to about 180 mg/kg, about 10 mg/kg to about 160 mg/kg, about 10 mg/kg to about 140 mg/kg, about 10 mg/kg to about 120 mg/kg, about 10 mg/kg to about 100 mg/kg, about 10 mg/kg to about 80 mg/kg, about 10 mg/kg to about 60 mg/kg, about 10 mg/kg to about 40 mg/kg, about 10 mg/kg to about 20 mg/kg, about 20 mg/kg to about 400 mg/kg, about 20 mg/kg to about 380 mg/kg, about 20 mg/kg to about 360 mg/kg, about 20 mg/kg to about 340 mg/kg, about 20 mg/kg to about 320 mg/kg, about 20 mg/kg to about 300 mg/kg, about 20 mg/kg to about 280 mg/kg, about 20 mg/kg to about 260 mg/kg, about 20 mg/kg to about 240 mg/kg, about 20 mg/kg to about 220 mg/kg, about 20 mg/kg to about 200 mg/kg, about 20 mg/kg to about 180 mg/kg, about 20 mg/kg to about 160 mg/kg, about 20 mg/kg to about 140 mg/kg, about 20 mg/kg to about 120 mg/kg, about 20 mg/kg to about 100 mg/kg, about 20 mg/kg to about 80 mg/kg, about 20 mg/kg to about 60 mg/kg, about 20 mg/kg to about 40 mg/kg, about 40 mg/kg to about 400 mg/kg, about 40 mg/kg to about 380 mg/kg, about 40 mg/kg to about 360 mg/kg, about 40 mg/kg to about 340 mg/kg, about 40 mg/kg to about 320 mg/kg, about 40 mg/kg to about 300 mg/kg, about 40 mg/kg to about 280 mg/kg, about 40 mg/kg to about 260 mg/kg, about 40 mg/kg to about 240 mg/kg, about 40 mg/kg to about 220 mg/kg, about 40 mg/kg to about 200 mg/kg, about 40 mg/kg to about 180 mg/kg, about 40 mg/kg to about 160 mg/kg, about 40 mg/kg to about 140 mg/kg, about 40 mg/kg to about 120 mg/kg, about 40 mg/kg to about 100 mg/kg, about 40 mg/kg to about 80 mg/kg, about 40 mg/kg to about 60 mg/kg, about 60 mg/kg to about 400 mg/kg, about 60 mg/kg to about 380 mg/kg, about 60 mg/kg to about 360 mg/kg, about 60 mg/kg to about 340 mg/kg, about 60 mg/kg to about 320 mg/kg, about 60 mg/kg to about 300 mg/kg, about 60 mg/kg to about 280 mg/kg, about 60 mg/kg to about 260 mg/kg, about 60 mg/kg to about 240 mg/kg, about 60 mg/kg to about 220 mg/kg, about 60 mg/kg to about 200 mg/kg, about 60 mg/kg to about 180 mg/kg, about 60 mg/kg to about 160 mg/kg, about 60 mg/kg to about 140 mg/kg, about 60 mg/kg to about 120 mg/kg, about 60 mg/kg to about 100 mg/kg, about 60 mg/kg to about 80 mg/kg, about 80 mg/kg to about 400 mg/kg, about 80 mg/kg to about 380 mg/kg, about 80 mg/kg to about 360 mg/kg, about 80 mg/kg to about 340 mg/kg, about 80 mg/kg to about 320 mg/kg, about 80 mg/kg to about 300 mg/kg, about 80 mg/kg to about 280 mg/kg, about 80 mg/kg to about 260 mg/kg, about 80 mg/kg to about 240 mg/kg, about 80 mg/kg to about 220 mg/kg, about 80 mg/kg to about 200 mg/kg, about 80 mg/kg to about 180 mg/kg, about 80 mg/kg to about 160 mg/kg, about 80 mg/kg to about 140 mg/kg, about 80 mg/kg to about 120 mg/kg, about 80 mg/kg to about 100 mg/kg, about 100 mg/kg to about 400 mg/kg, about 100 mg/kg to about 380 mg/kg, about 100 mg/kg to about 360 mg/kg, about 100 mg/kg to about 340 mg/kg, about 100 mg/kg to about 320 mg/kg, about 100 mg/kg to about 300 mg/kg, about 100 mg/kg to about 280 mg/kg, about 100 mg/kg to about 260 mg/kg, about 100 mg/kg to about 240 mg/kg, about 100 mg/kg to about 220 mg/kg, about 100 mg/kg to about 200 mg/kg, about 100 mg/kg to about 180 mg/kg, about 100 mg/kg to about 160 mg/kg, about 100 mg/kg to about 140 mg/kg, about 100 mg/kg to about 120 mg/kg, about 120 mg/kg to about 400 mg/kg, about 120 mg/kg to about 380 mg/kg, about 120 mg/kg to about 360 mg/kg, about 120 mg/kg to about 340 mg/kg, about 120 mg/kg to about 320 mg/kg, about 120 mg/kg to about 300 mg/kg, about 120 mg/kg to about 280 mg/kg, about 120 mg/kg to about 260 mg/kg, about 120 mg/kg to about 240 mg/kg, about 120 mg/kg to about 220 mg/kg, about 120 mg/kg to about 200 mg/kg, about 120 mg/kg to about 180 mg/kg, about 120 mg/kg to about 160 mg/kg, about 120 mg/kg to about 140 mg/kg, about 140 mg/kg to about 400 mg/kg, about 140 mg/kg to about 380 mg/kg, about 140 mg/kg to about 360 mg/kg, about 140 mg/kg to about 340 mg/kg, about 140 mg/kg to about 320 mg/kg, about 140 mg/kg to about 300 mg/kg, about 140 mg/kg to about 280 mg/kg, about 140 mg/kg to about 260 mg/kg, about 140 mg/kg to about 240 mg/kg, about 140 mg/kg to about 220 mg/kg, about 140 mg/kg to about 200 mg/kg, about 140 mg/kg to about 180 mg/kg, about 140 mg/kg to about 160 mg/kg, about 160 mg/kg to about 400 mg/kg, about 160 mg/kg to about 380 mg/kg, about 160 mg/kg to about 360 mg/kg, about 160 mg/kg to about 340 mg/kg, about 160 mg/kg to about 320 mg/kg, about 160 mg/kg to about 300 mg/kg, about 160 mg/kg to about 280 mg/kg, about 160 mg/kg to about 260 mg/kg, about 160 mg/kg to about 240 mg/kg, about 160 mg/kg to about 220 mg/kg, about 160 mg/kg to about 200 mg/kg, about 160 mg/kg to about 180 mg/kg, about 180 mg/kg to about 400 mg/kg, about 180 mg/kg to about 380 mg/kg, about 180 mg/kg to about 360 mg/kg, about 180 mg/kg to about 340 mg/kg, about 180 mg/kg to about 320 mg/kg, about 180 mg/kg to about 300 mg/kg, about 180 mg/kg to about 280 mg/kg, about 180 mg/kg to about 260 mg/kg, about 180 mg/kg to about 240 mg/kg, about 180 mg/kg to about 220 mg/kg, about 180 mg/kg to about 200 mg/kg, about 200 mg/kg to about 400 mg/kg, about 200 mg/kg to about 380 mg/kg, about 200 mg/kg to about 360 mg/kg, about 200 mg/kg to about 340 mg/kg, about 200 mg/kg to about 320 mg/kg, about 200 mg/kg to about 300 mg/kg, about 200 mg/kg to about 280 mg/kg, about 200 mg/kg to about 260 mg/kg, about 200 mg/kg to about 240 mg/kg, about 200 mg/kg to about 220 mg/kg, about 220 mg/kg to about 400 mg/kg, about 220 mg/kg to about 380 mg/kg, about 220 mg/kg to about 360 mg/kg, about 220 mg/kg to about 340 mg/kg, about 220 mg/kg to about 320 mg/kg, about 220 mg/kg to about 300 mg/kg, about 220 mg/kg to about 280 mg/kg, about 220 mg/kg to about 260 mg/kg, about 220 mg/kg to about 240 mg/kg, about 240 mg/kg to about 400 mg/kg, about 240 mg/kg to about 380 mg/kg, about 240 mg/kg to about 360 mg/kg, about 240 mg/kg to about 340 mg/kg, about 240 mg/kg to about 320 mg/kg, about 240 mg/kg to about 300 mg/kg, about 240 mg/kg to about 280 mg/kg, about 240 mg/kg to about 260 mg/kg, about 260 mg/kg to about 400 mg/kg, about 260 mg/kg to about 380 mg/kg, about 260 mg/kg to about 360 mg/kg, about 260 mg/kg to about 340 mg/kg, about 260 mg/kg to about 320 mg/kg, about 260 mg/kg to about 300 mg/kg, about 260 mg/kg to about 280 mg/kg, about 280 mg/kg to about 400 mg/kg, about 280 mg/kg to about 380 mg/kg, about 280 mg/kg to about 360 mg/kg, about 280 mg/kg to about 340 mg/kg, about 280 mg/kg to about 320 mg/kg, about 280 mg/kg to about 300 mg/kg, about 300 mg/kg to about 400 mg/kg, about 300 mg/kg to about 380 mg/kg, about 300 mg/kg to about 360 mg/kg, about 300 mg/kg to about 340 mg/kg, about 300 mg/kg to about 320 mg/kg, about 320 mg/kg to about 400 mg/kg, about 320 mg/kg to about 380 mg/kg, about 320 mg/kg to about 360 mg/kg, about 320 mg/kg to about 340 mg/kg, about 340 mg/kg to about 400 mg/kg, about 340 mg/kg to about 380 mg/kg, about 340 mg/kg to about 360 mg/kg, about 360 mg/kg to about 400 mg/kg, about 360 mg/kg to about 380 mg/kg, or about 380 mg/kg to about 400 mg/kg) of body weight of a phenylbutyrate compound (e.g., any of the phenylbutyrate compounds described herein or known in the art, e.g., sodium phenylbutyrate).

In some embodiments, the bile acid (e.g., TURSO) is administered in an amount of about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, or about 70 mg/kg of body weight. In some embodiments, the phenylbutyrate compound (e.g., sodium phenylbutyrate) is administered in an amount of about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 120 mg/kg, about 140 mg/kg, about 160 mg/kg, about 180 mg/kg, about 200 mg/kg, about 220 mg/kg, about 240 mg/kg, about 260 mg/kg, about 280 mg/kg, about 300 mg/kg, about 320 mg/kg, about 340 mg/kg, about 360 mg/kg, about 380 mg/kg, or about 400 mg/kg of body weight.

The bile acid or a pharmaceutically acceptable salt thereof and the phenylbutyrate compound can be administered separately or concurrently, including as a part of a regimen of treatment. The compounds can be administered daily, weekly, monthly, or quarterly. In some embodiments, the compounds are administered once a day, twice a day, or three times a day or more. The compounds can be administered over a period of weeks, months, or years. For example, the compounds can be administered over a period of at least or about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 60 weeks, 70 weeks, 80 weeks, 90 weeks, 100 weeks, 105 weeks, or more. In some embodiments, the compounds can be administered over a period of at least or about 1 month, 2 months, 3 months, 4 months, 5 months, 6 months, 7 months, 8 months, 9 months, 10 months, 11 months, 1 year, 2 years, 3 years, 4 years, or at least or about years, or more. The bile acid and phenylbutyrate compound can, for example, be administered once a day or twice a day for 60 days or less (e.g., 55 days, 50 days, 45 days, 40 days, 35 days, 30 days or less). Alternatively, the bile acid and phenylbutyrate compounds can be administered once a day or twice a day for more than 60 days (e.g., more than 65, 70, 75, 80, 85, 90, 95, 100, 105, 110, 115, 120, 130, 140, 150, 160, 180, 200, 250, 300, 400, 500, 600 days).

In some embodiments of any of the methods described herein, the bile acid is TURSO. TURSO can be administered to a subject at a dose of about 0.5 grams to about 10 grams daily (e.g., about 1, 2, 3, 4, 5, 6, 7, 8, or 9 grams daily). For example, TURSO can be administered at an amount of about 0.5 to about 5 grams (e.g., about 0.5 to about 4.5, about 0.5 to about 4, about 0.5 to about 3.5, about 0.5 to about 3, about 0.5 to about 2.5, about 0.5 to about 2, about 0.5 to about 1.5, about 0.5 to about 1, about 1 to about 5, about 1 to about 4.5, about 1 to about 4, about 1 to about 3.5, about 1 to about 3, about 1 to about 2.5, about 1 to about 2, about 1 to about 1.5, about 1.5 to about 5, about 1.5 to about 4.5, about 1.5 to about 4, about 1.5 to about 3.5, about 1.5 to about 3, about 1.5 to about 2.5, about 1.5 to about 2, about 2 to about 5, about 2 to about 4.5, about 2 to about 4, about 2 to about 3.5, about 2 to about 3, about 2 to about 2.5, about 2.5 to about 5, about 2.5 to about 4.5, about 2.5 to about 4, about 2.5 to about 3.5, about 2.5 to about 3, about 3 to about 5, about 3 to about 4.5, about 3 to about 4, about 3 to about 3.5, about 3.5 to about 5 about 3.5 to about 4.5, about 3.5 to about 4, about 4 to about 5, about 4 to about 4.5, or about 4.5 to about 5 grams) per day. In some embodiments, TURSO is administered to a subject at an amount of about 1 gram per day. In some embodiments, TURSO is administered to a subject at an amount of about 2 grams per day. For example, TURSO can be administered at an amount of about 1 gram twice a day.

In some embodiments of any of the methods described herein, the phenylbutyrate compound is sodium phenylbutyrate. Sodium phenylbutyrate can be administered at an amount of about 1 gram to about 30 grams daily (e.g., about 2, 3, 4, 5, 6, 7, 8, 9, 10, 11, 12, 13, 14, 15, 16, 17, 18, 19, 20, 21, 22, 23, 24, 25, 26, 27, 28, or 29 grams daily). For example, sodium phenylbutyrate can be administered at an amount of about 0.5 to about 10 grams (e.g., about 0.5 to about 9.5, about 0.5 to about 9, about 0.5 to about 8.5, about 0.5 to about 8, about 0.5 to about 7.5, about 0.5 to about 7, about 0.5 to about 6.5, about 0.5 to about 6, about 0.5 to about 5.5, about 0.5 to about 5, about 0.5 to about 4.5, about 0.5 to about 4, about 0.5 to about 3.5, about 0.5 to about 3, about 0.5 to about 2.5, about 0.5 to about 2, about 0.5 to about 1.5, about 0.5 to about 1, about 1 to about 10, about 1 to about 9.5, about 1 to about 9, about 1 to about 8.5, about 1 to about 8, about 1 to about 7.5, about 1 to about 7, about 1 to about 6.5, about 1 to about 6, about 1 to about 5.5, about 1 to about 5, about 1 to about 4.5, about 1 to about 4, about 1 to about 3.5, about 1 to about 3, about 1 to about 2.5, about 1 to about 2, about 1 to about 1.5, about 1.5 to about 10, about 1.5 to about 9.5, about 1.5 to about 9, about 1.5 to about 8.5, about 1.5 to about 8, about 1.5 to about 7.5, about 1.5 to about 7, about 1.5 to about 6.5, about 1.5 to about 6, about 1.5 to about 5.5, about 1.5 to about 5, about 1.5 to about 4.5, about 1.5 to about 4, about 1.5 to about 3.5, about 1.5 to about 3, about 1.5 to about 2.5, about 1.5 to about 2, about 2 to about 10, about 2 to about 9.5, about 2 to about 9, about 2 to about 8.5, about 2 to about 8, about 2 to about 7.5, about 2 to about 7, about 2 to about 6.5, about 2 to about 6, about 2 to about 5.5, about 2 to about 5, about 2 to about 4.5, about 2 to about 4, about 2 to about 3.5, about 2 to about 3, about 2 to about 2.5, about 2.5 to about 10, about 2.5 to about 9.5, about 2.5 to about 9, about 2.5 to about 8.5, about 2.5 to about 8, about 2.5 to about 7.5, about 2.5 to about 7, about 2.5 to about 6.5, about 2.5 to about 6, about 2.5 to about 5.5, about 2.5 to about 5, about 2.5 to about 4.5, about 2.5 to about 4, about 2.5 to about 3.5, about 2.5 to about 3, about 3 to about 10, about 3 to about 9.5, about 3 to about 9, about 3 to about 8.5, about 3 to about 8, about 3 to about 7.5, about 3 to about 7, about 3 to about 6.5, about 3 to about 6, about 3 to about 5.5, about 3 to about 5, about 3 to about 4.5, about 3 to about 4, about 3 to about 3.5, about 3.5 to about 10, about 3.5 to about 9.5, about 3.5 to about 9, about 3.5 to about 8.5, about 3.5 to about 8, about 3.5 to about 7.5, about 3.5 to about 7, about 3.5 to about 6.5, about 3.5 to about 6, about 3.5 to about 5.5, about 3.5 to about 5, about 3.5 to about 4.5, about 3.5 to about 4, about 4 to about 10, about 4 to about 9.5, about 4 to about 9, about 4 to about 8.5, about 4 to about 8, about 4 to about 7.5, about 4 to about 7, about 4 to about 6.5, about 4 to about 6, about 4 to about 5.5, about 4 to about 5, about 4 to about 4.5, about 4.5 to about 10, about 4.5 to about 9.5, about 4.5 to about 9, about 4.5 to about 8.5, about 4.5 to about 8, about 4.5 to about 7.5, about 4.5 to about 7, about 4.5 to about 6.5, about 4.5 to about 6, about 4.5 to about 5.5, about 4.5 to about 5, about 5 to about 10, about 5 to about 9.5, about 5 to about 9, about 5 to about 8.5, about 5 to about 8, about 5 to about 7.5, about 5 to about 7, about 5 to about 6.5, about 5 to about 6, about 5 to about 5.5, about 5.5 to about 10, about 5.5 to about 9.5, about 5.5 to about 9, about 5.5 to about 8.5, about 5.5 to about 8, about 5.5 to about 7.5, about 5.5 to about 7, about 5.5 to about 6.5, about 5.5 to about 6, about 6 to about 10, about 6 to about 9.5, about 6 to about 9, about 6 to about 8.5, about 6 to about 8, about 6 to about 7.5, about 6 to about 7, about 6 to about 6.5, about 6.5 to about 10, about 6.5 to about 9.5, about 6.5 to about 9, about 6.5 to about 8.5, about 6.5 to about 8, about 6.5 to about 7.5, about 6.5 to about 7, about 7 to about 10, about 7 to about 9.5, about 7 to about 9, about 7 to about 8.5, about 7 to about 8, about 7 to about 7.5, about 7.5 to about 10, about 7.5 to about 9.5, about 7.5 to about 9, about 7.5 to about 8.5, about 7.5 to about 8, about 8 to about 10, about 8 to about 9.5, about 8 to about 9, about 8 to about 8.5, about 8.5 to about 10, about 8.5 to about 9.5, about 8.5 to about 9, about 9 to about 10, about 9 to about 9.5, or about 9.5 to about 10 grams) per day. In some embodiments, sodium phenylbutyrate is administered at an amount of about 3 grams per day. In some embodiments, sodium phenylbutyrate is administered at an amount of about 6 grams per day. For example, sodium phenylbutyrate can be administered at an amount of about 3 grams twice a day. In some embodiments, the bile acid and phenylbutyrate compound are administered at a ratio by weight of about 2.5:1 to about 3.5:1 (e.g., about 3:1).

In some embodiments of any of the methods described herein, the methods include administering TURSO and sodium phenylbutyrate to the subject according to a first regimen followed by a second regimen, where the first regimen includes administering about 1 gram of TURSO once a day and about 3 grams of sodium phenylbutyrate once a day for at least about 14 days (e.g., at least 16, 17, 18, 19, 20, 21, 22, 23, 24, 25, 26, 27, 28, 29, 30, 35, or 40 days; or for about two weeks; or for about three weeks), and the second regimen includes administering about 1 gram of TURSO twice a day and about 3 grams of sodium phenylbutyrate twice a day. In some embodiments, the second regimen is for at least about 30 days (e.g., at least 35, 40, 45, 50, 60, 80, 100, 120, 150, 180, 250, 300, or 400 days).

In some embodiments of any of the methods described herein, the subject is diagnosed with PSP, at risk for developing PSP, or suspected as having PSP. The subject may, for example, have been diagnosed with PSP for 24 months or less (e.g., any of the subranges within this range described herein). For example, the subject may have been diagnosed with PSP for 1 week or less, or on the same day that the presently disclosed treatments are administered. The subject may have shown one or more symptoms of PSP for 24 months or less (e.g., any of the subranges within this range described herein), has elevated levels of total tau, phospho-tau, neurofilament-light (NfL), or YKL-40.

In some embodiments, prior to treatment the subjects have a baseline CSF total tau level of about 300 pg/mL or higher (e.g., about 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 3000, 3200, 3500, 3800, or 4000 pg/mL or higher). In some embodiments, administration of the bile acid (e.g., TURSO) and the phenylbutyrate compound (e.g., sodium phenylbutyrate) reduces the levels of CSF total tau by about 35 pg/mL or more (e.g., about 40, 45, 50, 55, 60, 65, 70, 75, 80, 85, 90, 95 pg/mL or more).

In some embodiments, prior to treatment the subjects have a baseline CSF phospho-tau (e.g., phospho-tau 181, phospho-tau 199, and/or phospho-tau 231) level of about 30 pg/mL or higher. In some embodiments, prior to treatment the subjects have a baseline CSF phospho-Tau (e.g., phospho-tau 181) level of about 70 pg/mL or higher (e.g., about 75, 100, 125, 150, 175, or 200 pg/mL or higher). In some embodiments, administration of the bile acid (e.g., TURSO) and the phenylbutyrate compound (e.g., sodium phenylbutyrate) reduces the levels of CSF phospho-tau by about 5 pg/mL or more (e.g., about 10, 15, 20, 25, 30, 35, 40, 45, 50, 55, 60 pg/mL or more). In some embodiments, prior to treatment the subjects have a baseline CSF Fatty acid-binding protein 3 (FABP3) level of about 2000 pg/mL or higher (e.g., about 2200, 2500, 2800, 3200, 3500, 3800, 3900, 4000, 4100, or 4200 pg/mL or higher). In some embodiments, administration of the bile acid (e.g., TURSO) and the phenylbutyrate compound (e.g., sodium phenylbutyrate) reduces the levels of CSF FABP3 by about 200 pg/mL or more (e.g., about 250, 280, 310, 320, 330, 340, 350, 360, 370, 380, 390, 400, 410, 420, 430, 440, 450, 460, 470, 480, 490, 500 pg/mL or more).

In some embodiments, prior to treatment the subjects have a baseline CSF neurogranin level of 200 pg/mL or higher (e.g., about 250, 300, 350, 400, 450, 500, 550, 600, 650, 700, 750, 800, 850, 900, 950, 1000 pg/mL or higher). In some embodiments, administration of the bile acid (e.g., TURSO) and the phenylbutyrate compound (e.g., sodium phenylbutyrate) reduces the levels of CSF neurogranin by about 30 pg/mL or more (e.g., about 35, 40, 45, 50, 55, 60, 70, 80, 90, 100, 200 pg/mL or more).

In some embodiments, prior to treatment the subjects have a baseline CSF YKL-40 level of 140000 pg/mL or higher (e.g., about 160000, 180000, 210000, 220000, 230000, 240000, 250000, 300000 pg/mL or higher). In some embodiments, administration of the bile acid (e.g., TURSO) and the phenylbutyrate compound (e.g., sodium phenylbutyrate) reduces the levels of CSF YKL-40 by about 5000 pg/mL or more (e.g., about 7000, 9000, 11000, 12000, 13000, 14000, 15000, 16000, 18000, 20000, 25000 pg/mL or more).

In some embodiments, prior to treatment the subjects have a baseline CSF IL-15 level of about 1 to about 5 pg/mL (e.g., about 1.5, 2, 2.5, 3, 3.5, 4 or 4.5 pg/mL). In some embodiments, administration of the bile acid (e.g., TURSO) and the phenylbutyrate compound (e.g., sodium phenylbutyrate) reduces the levels of CSF IL-15 by about 0.01 pg/mL or more (e.g., about 0.02, 0.03, 0.04, 0.05, 0.06, 0.07, 0.08, 0.09, 0.1, 0.2, 0.3, 0.4, 0.5 pg/mL or more).

In some embodiments, the CSF Aβ₁₋₄₂ level is about 500 pg/mL or lower (e.g., about 450, 400, 350, 300, 250, 200, 150, 100, 50, or 25 pg/mL, or lower). The subject may have a baseline CSF Aβ₁₋₄₂ level of about 150 to about 550 pg/mL and/or a baseline CSF Aβ₁₋₄₀ level of about 3500 to about 9500 pg/mL. Administration of the bile acid (e.g., TURSO) and the phenylbutyrate compound (e.g., sodium phenylbutyrate) can increase the Aβ₁₋₄₂/Aβ₁₋₄₀ ratio by about 0.001 to about 0.02 (e.g., about 0.002 to about 0.015, or about 0.009).

The subject can have a baseline 8-OHdG level of about 2 to about 5 pg/mL (e.g., about 2.5, 3, 3.5, 4, or 4.5 pg/mL). Administration of the bile acid (e.g., TURSO) and the phenylbutyrate compound (e.g., sodium phenylbutyrate) can increase the 8-OHdG level by about 0.1 pg/mL or more (e.g., about 0.2, 0.3, 0.4, 0.5, 0.6, or 0.7 pg/mL or more).

Methods described in the present disclosure can include treatment of a neurodegenerative disease (e.g., a tauopathy like PSP) per se, as well as treatment for one or more symptoms of a neurodegenerative disease (e.g., a tauopathy like PSP). “Treating” a neurodegenerative disease (e.g., a tauopathy like PSP) does not require 100% abolition of the disease or disease symptoms in the subject. Any relief or reduction in the severity of symptoms or features of the disease is contemplated. “Treating” a neurodegenerative disease (e.g., a tauopathy like PSP) also refers to a delay in onset of symptoms (e.g., in prophylaxis treatment) or delay in progression of symptoms or the loss of function associated with the disease. “Treating” a neurodegenerative disease (e.g., a tauopathy like PSP) also refers to eliminating or reducing one or more side effects of a treatment (e.g., those caused by any of the therapeutic agents for treating a neurodegenerative disease (e.g., a tauopathy like PSP) disclosed herein or known in the art). “Treating” a neurodegenerative disease (e.g., a tauopathy like PSP) also refers to eliminating or reducing one or more direct or indirect effects of a neurodegenerative disease (e.g., a tauopathy like PSP) disease progression. The subject may not exhibit signs of a neurodegenerative disease (e.g., a tauopathy like PSP) but may be at risk for a neurodegenerative disease (e.g., a tauopathy like PSP). For instance, the subject may carry mutations in genes associated with a neurodegenerative disease (e.g., a tauopathy like PSP), have elevated biomarker levels suggesting a risk of developing a neurodegenerative disease (e.g., a tauopathy like PSP) (e.g., but not limited to, total tau, phospho-tau, or YKL-40). The subject may exhibit early signs of the disease or display symptoms of established or progressive disease. The disclosure contemplates any degree of delay in the onset of symptoms, alleviation of one or more symptoms of the disease, or delay in the progression of any one or more disease symptoms.

The treatment provided in the present disclosure can be initiated at any stage during disease progression. For example, treatment can be initiated prior to onset (e.g., for subjects at risk for developing a neurodegenerative disease (e.g., a tauopathy like PSP), for instance, those with elevated total tau or phospho-tau), at symptom onset or immediately following detection of a neurodegenerative disease (e.g., a tauopathy like PSP) symptoms, upon observation of any one or more symptoms (e.g., decline in cognitive functions) that would lead a skilled practitioner to suspect that the subject may be developing a neurodegenerative disease (e.g., a tauopathy like PSP). Treatment can also be initiated at later stages.

Treatment methods can include a single administration, multiple administrations, and repeating administration as required for the prophylaxis or treatment of a neurodegenerative disease (e.g., a tauopathy like PSP), or at least one symptom of a neurodegenerative disease (e.g., a tauopathy like PSP). The duration of prophylaxis treatment can be a single dosage or the treatment may continue (e.g., multiple dosages), e.g., for years or indefinitely for the lifespan of the subject. For example, a subject at risk for a neurodegenerative disease (e.g., a tauopathy like PSP) may be treated with the methods provided herein for days, weeks, months, or even years so as to prevent the disease from occurring or fulminating. In some embodiments treatment methods can include assessing a level of disease in the subject prior to treatment, during treatment, and/or after treatment. The treatment provided herein can be administered one or more times daily, or it can be administered weekly or monthly. In some embodiments, treatment can continue until a decrease in the level of disease in the subject is detected. The methods provided herein may in some embodiments begin to show efficacy (e.g., alleviating one or more symptoms of a neurodegenerative disease (e.g., a tauopathy like PSP), improvement as measured by a cognitive test, less than 60 days (e.g., less than 50, 45, 40, 35, 30, 25, 20, 15, or 10 days) after the initial administration, or after less than 60 administrations (e.g., less than 50, 45, 40, 35, 30, 25, 20, 15, or 10 administrations).

The terms “administer”, “administering”, or “administration” as used herein refers to administering drugs described herein to a subject using any art-known method, e.g., ingesting, injecting, implanting, absorbing, or inhaling, the drug, regardless of form. In some embodiments, one or more of the compounds disclosed herein can be administered to a subject by ingestion orally and/or topically (e.g., nasally). For example, the methods herein include administration of an effective amount of compound or compound composition to achieve the desired or stated effect. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Following administration, the subject can be evaluated to detect, assess, or determine their level of disease. In some embodiments, treatment can continue until a change (e.g., reduction) in the level of disease in the subject is detected.

Upon improvement of a patient's condition (e.g., a change (e.g., decrease) in the level of disease in the subject), a maintenance dose of a compound, composition or combination of this disclosure may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

### Symptom and Outcome Measurements

Methods of evaluating symptoms, monitoring a neurodegenerative disease, such as PSP, progression and/or evaluating the subject's response to the treatment methods are described herein. Non-limiting examples include physical evaluation by a physician, cognitive tests (e.g., PSP Rating Scale, Movement Disorder Society-Unified Parkinson's Disease Rating Scale Part II, Schwab and England Activities of Daily Living Scale, EuroQuality of Life, Zarit Burden Interview, Clinical Global Impression of Severity and Change, Mini-Mental State Exam, Montreal Cognitive Assessment, Columbia-Suicide Severity Rating Scale, or any other suitable test known to one skilled in the art), measurement of one or more CSF biomarkers (e.g., total tau (t-tau), phospho-tau 181 (e.g., p-tau 181 or another phospho-tau), neurofilament-light (NfL), Ubiquitin carboxyl-terminal hydrolase L1 (UCHL1)/PGP9.5, Glial fibrillary acidic protein (GFAP), 8-hydoxy-2′-deoxyguanosine (8-OHdG), Soluble insulin receptor (sIR), Aβ₁₋₄₂, Aβ₁₋₄₀, Aβ₁₋₄₂/Aβ₁₋₄₀ ratio, leptin, 24-hydroxycholesterol, YKL-40), neuroimaging (e.g., measuring hippocampal volume, grey matter, average cortical thickness, number of white matter lesions, white matter lesion volume, ventricular volume through known methods, such as, MRI, Lumbar Puncture, CT, SPECT, FDG-PET, or DIT), or a combination of any of these methods (e.g., combination of a cognitive test and the level of one or more CSF biomarker). Also contemplated are overall burden, quality of life (i.e., subject quality of life and caregiver burden), and survival.

In some embodiments, the methods described herein result in an improvement in score received in one or more cognitive test. In other embodiments, the methods described herein result in a significant decrease in t-tau, phospho-tau, or YKL-40 (for example, as measured in the blood or CSF). In another example, the methods described herein result in an increase in Aβ₁₋₄₂/Aβ₁₋₄₀ (for example, as measured in the blood CSF; see e.g., Lewczuk P, Lelental N, Spitzer P, Maler J M, Komhuber J. Amyloid-β 42/40 cerebrospinal fluid concentration ratio in the diagnostics of Alzheimer's disease: validation of two novel assays. J Alzheimers Dis. 2015; 43(1):183-91. doi: 10.3233/JAD-140771. PMID: 25079805; Hansson, O., Lehmann, S., Otto, M. et al. Advantages and disadvantages of the use of the CSF Amyloid β (Aβ) 42/40 ratio in the diagnosis of Alzheimer's Disease. Alz Res Therapy 11, 34 (2019). https://doi.org/10.1186/si3195-019-0485-0 and James D. Doecke, Virginia Pérez-Grijalba, Noelia Fandos, Christopher Fowler, Victor L. Villemagne, Colin L. Masters, Pedro Pesini, Manuel Sarasa, for the AIBL Research Group, “Total Aβ42/Aβ40 ratio in plasma predicts amyloid-PET status, independent of clinical AD diagnosis,” Neurology Apr 2020, 94 (15) e1580-e1591; DOI: 10.1212/WNL.0000000000009240; incorporated herein by reference). In some embodiments, the methods described herein result in a significant increase in ventricular volume (for example, as measured by an imaging method, such as, MRI). In some embodiments, the methods described herein result in an increase in hippocampal volume, grey matter, average cortical thickness, and/or a decrease in the number of white matter lesions (for example, as measured by an imaging method, such as, MRI).

In some embodiments of any of the methods described herein, the methods include administering TURSO and sodium phenylbutyrate to the subject according to a first regimen followed by a second regimen, where the first regimen includes administering about 1 gram of TURSO once a day and about 3 grams of sodium phenylbutyrate once a day for at least about 14 days (e.g., at least 16, 17, 18, 19, 20, 21, 22, 23, 24, 25, 26, 27, 28, 29, 30, 35, or 40 days; or for about two weeks; or for about three weeks), and the second regimen includes administering about 1 gram of TURSO twice a day and about 3 grams of sodium phenylbutyrate twice a day (e.g., for more than 1 day). In some embodiments, the method of administering TURSO and sodium phenylbutyrate to the subject according to a first regimen (e.g., at least 16, 17, 18, 19, 20, 21, 22, 23, 24, 25, 26, 27, 28, 29, 30, 35, or 40 days; or for about two weeks; or for about three weeks) followed by a second regimen (e.g., about 20 weeks, about 21 weeks, or about 22 weeks) is for at least 24 weeks. If treatment for 24 weeks shows an improvement in the subject's PSPRS score as compared the subject's PSPRS score at the start of treatment, treatment with the second regimen (administering about 1 gram of TURSO twice a day and about 3 grams of sodium phenylbutyrate twice a day) is continued. In some embodiments, in addition to assessing the subject's PSPRS score, other outcome measures (e.g., Movement Disorder Society-Unified Parkinson's Disease Rating Scale Part II, Schwab and England Activities of Daily Living Scale, EuroQuality of Life, Zarit Burden Interview, Clinical Global Impression of Severity and Change, Mini-Mental State Exam, Montreal Cognitive Assessment, Columbia-Suicide Severity Rating Scale or any plasma/CSF biomarkers) can also be used to determine if the treatment regimen is resulting in an improvement in the subject's condition.

### Composition

The present disclosure provides methods of treating at least one symptom of a neurodegenerative disease (such as PSP) in a subject, the methods including administering to the subject a bile acid or a pharmaceutically acceptable salt thereof and a phenylbutyrate compound. In some embodiments, the methods include administering a composition comprising a TURSO and a sodium phenylbutyrate to a subject.

### Bile Acid

As used herein, “bile acid” refers to naturally occurring surfactants having a nucleus derived from cholanic acid substituted with a 3α-hydroxyl group and optionally with other hydroxyl groups as well, typically at the C6, C7 or C12 position of the sterol nucleus. Bile acid derivatives (e.g., aqueous soluble bile acid derivatives) and bile acids conjugated with an amine are also encompassed by the term “bile acid”. Bile acid derivatives include, but are not limited to, derivatives formed at the carbon atoms to which hydroxyl and carboxylic acid groups of the bile acid are attached with other functional groups, including but not limited to halogens and amino groups. Soluble bile acids may include an aqueous preparation of a free acid form of bile acids combined with one of HCl, phosphoric acid, citric acid, acetic acid, ammonia, or arginine. Suitable bile acids include but are not limited to, taurursodiol (TURSO), ursodeoxycholic acid (UDCA), chenodeoxycholic acid (also referred to as “chenodiol” or “chenic acid”), cholic acid, hyodeoxycholic acid, deoxycholic acid, 7-oxolithocholic acid, lithocholic acid, iododeoxycholic acid, iocholic acid, taurochenodeoxycholic acid, taurodeoxycholic acid, glycoursodeoxycholic acid, taurocholic acid, glycocholic acid, or an analog, derivative, or prodrug thereof.

In some embodiments, the bile acids of the present disclosure are hydrophilic bile acids. Hydrophilic bile acids include but are not limited to, TURSO, UDCA, chenodeoxycholic acid, cholic acid, hyodeoxycholic acid, lithocholic acid, and glycoursodeoxycholic acid. Pharmaceutically acceptable salts or solvates of any of the bile acids disclosed herein are also contemplated. In some embodiments, bases commonly employed to form pharmaceutically acceptable salts of the bile acids of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

The terms “tauroursodeoxycholic acid” (TUDCA) and “taurursodiol” (TURSO) are used interchangeably herein.

The bile acid described herein can be TURSO, as shown in formula I (with labeled carbons to assist in understanding where substitutions may be made). In some embodiments, the TURSO is a hydrate, such as TURSO dihydrate.

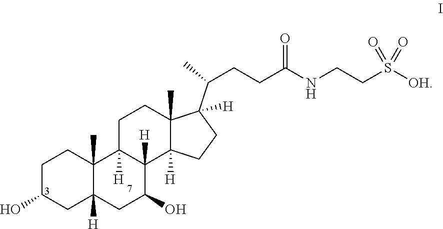

The bile acid described herein can be UDCA as shown in formula II (with labeled carbons to assist in understanding where substitutions may be made).

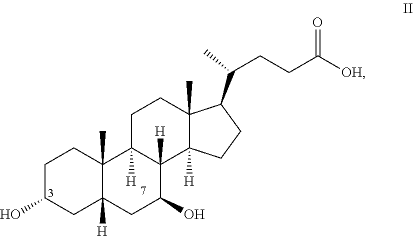

or a pharmaceutically acceptable salt thereof.

Derivatives of bile acids of the present disclosure can be physiologically related bile acid derivatives. For example, any combination of substitutions of hydrogen at position 3 or 7, a shift in the stereochemistry of the hydroxyl group at positions 3 or 7, in the formula of TURSO or UDCA are suitable for use in the present composition.

The “bile acid” can also be a bile acid conjugated with an amino acid. The amino acid in the conjugate can be, but are not limited to, taurine, glycine, glutamine, asparagine, methionine, or carbocysteine. Other amino acids that can be conjugated with a bile acid of the present disclosure include arginine, histidine, lysine, aspartic acid, glutamic acid, serine, threonine, cysteine, proline, alanine, valine, isoleucine, leucine, phenylalanine, tyrosine, and tryptophan, as well as β-alanine, and γ-aminobutyric acid. One example of such a bile acid is a compound of formula III:

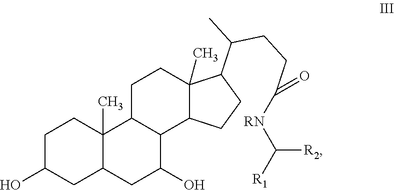

wherein
R is —H or C₁-C₄ alkyl;
R₁ is —CH₂—SO₃R₃, CH₂COOH, or CH₂CH₂COOH, and R₂ is —H;
or R₁ is —COOH and R₂ is —CH₂—CH₂—CONH₂, —CH₂—CONH₂, —CH₂—CH₂—SCH₃, CH₂CH₂CH₂NH(C═NH)NH₂, CH₂(imidazolyl), CH₂CH₂CH₂CH₂NH₂, CH₂COOH, CH₂CH₂COOH, CH₂OH, CH(OH)CH₃, CH₂SH, pyrrolidin-2-yl, CH₃, 2-propyl, 2-butyl, 2-methylbutyl, CH₂(phenyl), CH₂(4-OH-phenyl), or —CH₂—S—CH₂—COOH; and
R₃ is —H or the residue of an amino acid, or a pharmaceutically acceptable analog, derivative, prodrug thereof, or a mixture thereof. One example of the amino acid is a basic amino acid. Other examples of the amino acid include glycine, glutamine, asparagine, methionine, carbocysteine, arginine, histidine, lysine, aspartic acid, glutamic acid, serine, threonine, cysteine, proline, alanine, valine, isoleucine, leucine, phenylalanine, tyrosine, and tryptophan, as well as β-alanine, and γ-aminobutyric acid.

Another example of a bile acid of the present disclosure is a compound of formula IV:

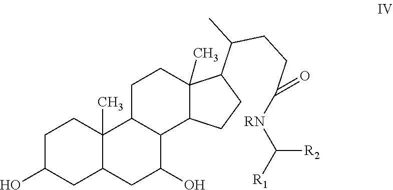

wherein
   R is —H or C₁-C₄ alkyl;
   R₁ is —CH₂—SO₃R₃, and R₂ is —H;
   or R₁ is —COOH and R₂ is —CH₂—CH₂—CONH₂, —CH₂—CONH₂, —CH₂—CH₂—SCH₃, or —CH₂—S—CH₂—COOH; and
   R₃ is —H or the residue of a basic amino acid, or a pharmaceutically acceptable analog, derivative, prodrug thereof, or a mixture thereof. Examples of basic amino acids include lysine, histidine, and arginine.

In some embodiments, the bile acid is TURSO. TURSO is an ambiphilic bile acid and is the taurine conjugate form of UDCA. TURSO recovers mitochondrial bioenergetic deficits through incorporating into the mitochondrial membrane, reducing Bax translocation to the mitochondrial membrane, reducing mitochondrial permeability, and increasing the apoptotic threshold of the cell (Rodrigues et al. Biochemistry 42, 10: 3070-3080, 2003). It is used for the treatment of cholesterol gallstones, where long periods of treatment is generally required (e.g., 1 to 2 years) to obtain complete dissolution. It has been used for the treatment of cholestatic liver diseases including primary cirrhosis, pediatric familial intrahepatic cholestasis and primary sclerosing cholangitis and cholestasis due to cystic fibrosis. TURSO is contraindicated in subjects with biliary tract infections, frequent biliary colic, or in subjects who have trouble absorbing bile acids (e.g., ileal disease or resection). Drug interactions may include with substances that inhibit the absorption of bile acids, such as cholestyramine, and with drugs that increase the elimination of cholesterol in the bile (TURSO reduces biliary cholesterol content). Based on similar physicochemical characteristics, similarities in drug toxicity and interactions exist between TURSO and UDCA. The most common adverse reactions reported with the use of TURSO (≥1%) are: abdominal discomfort, abdominal pain, diarrhea, nausea, pruritus, and rash. There are some cases of pruritus and a limited number of cases of elevated liver enzymes.

In some embodiments, the bile acid is UDCA. UDCA, or ursodiol, has been used for treating gallstones, and is produced and secreted endogenously by the liver as a taurine (TURSO) or glycine (GUDCA) conjugate. Taurine conjugation increases the solubility of UDCA by making it more hydrophilic. TURSO is taken up in the distal ileum under active transport and therefore likely has a slightly a longer dwell time within the intestine than UDCA which is taken up more proximally in the ileum. Ursodiol therapy has not been associated with liver damage. Abnormalities in liver enzymes have not been associated with Actigall® (Ursodiol USP capsules) therapy and, Actigall® has been shown to decrease liver enzyme levels in liver disease. However, subjects given Actigall® should have SGOT (AST) and SGPT (ALT) measured at the initiation of therapy and thereafter as indicated by the particular clinical circumstances. Previous studies have shown that bile acid sequestering agents such as cholestyramine and colestipol may interfere with the action of ursodiol by reducing its absorption. Aluminum-based antacids have been shown to adsorb bile acids in vitro and may be expected to interfere with ursodiol in the same manner as the bile acid sequestering agents. Estrogens, oral contraceptives, and clofibrate (and perhaps other lipid-lowering drugs) increase hepatic cholesterol secretion, and encourage cholesterol gallstone formation and hence may counteract the effectiveness of ursodiol.

### Phenylbutyrate Compounds

Phenylbutyrate compound is defined herein as encompassing phenylbutyrate (a low molecular weight aromatic carboxylic acid) as a free acid (4-phenylbutyrate (4-PBA), 4-phenylbutyric acid, or phenylbutyric acid), and pharmaceutically acceptable salts, co-crystals, polymorphs, hydrates, solvates, conjugates, derivatives or pro-drugs thereof. Phenylbutyrate compounds described herein also encompass analogs of 4-PBA, including but not limited to Glyceryl Tri-(4-phenylbutyrate), phenylacetic acid (which is the active metabolite of PBA), 2-(4-Methoxyphenoxy) acetic acid (2-POAA-OMe), 2-(4-Nitrophenoxy) acetic acid (2-POAA-NO2), and 2-(2-Naphthyloxy) acetic acid (2-NOAA), and their pharmaceutically acceptable salts. Phenylbutyrate compounds also encompass physiologically related 4-PBA species, such as but not limited to any substitutions for Hydrogens with Deuterium in the structure of 4-PBA. Other HDAC2 inhibitors are contemplated herein as substitutes for phenylbutyrate compounds.

Physiologically acceptable salts of phenylbutyrate, include, for example sodium, potassium, magnesium or calcium salts. Other example of salts include ammonium, zinc, or lithium salts, or salts of phenylbutyrate with an orgain amine, such as lysine or arginine.

In some embodiments of any of the methods described herein, the phenylbutyrate compound is sodium phenylbutyrate. Sodium phenylbutyrate has the following formula:

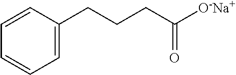

Phenylbutyrate is a pan-HDAC inhibitor and can ameliorate ER stress through upregulation of the master chaperone regulator DJ-1 and through recruitment of other chaperone proteins (See e.g., Zhou et al. J Biol Chem. 286: 14941-14951, 2011 and Suaud et al. JBC. 286:21239-21253, 2011). The large increase in chaperone production reduces activation of canonical ER stress pathways, folds misfolded proteins, and has been shown to increase survival in in vivo models including the G93A SOD1 mouse model of ALS (See e.g., Ryu, H et al. J Neurochem. 93:1087-1098, 2005).

### Formulation

Bile acids and phenylbutyrate compounds described herein can be formulated for use as or in pharmaceutical compositions. For example, the methods described herein can include administering an effective amount of a composition comprising TURSO and sodium phenylbutyrate. The term “effective amount”, as used herein, refer to an amount or a concentration of one or more drugs administered for a period of time (including acute or chronic administration and periodic or continuous administration) that is effective within the context of its administration for causing an intended effect or physiological outcome. The composition can include about 5% to about 15% w/w (e.g., about 6% to about 14%, about 7% to about 13%, about 8% to about 12%, about 8% to about 11%, about 9% to about 10%, or about 9.7% w/w) of TURSO and about 15% to about 45% w/w (e.g., about 20% to about 40%, about 25% to about 35%, about 28% to about 32%, or about 29% to about 30%, e.g., about 29.2% w/w) of sodium phenylbutyrate. In some embodiments, the composition includes about 9.7% w/w of TURSO and 29.2% w/w of sodium phenylbutyrate.

The sodium phenylbutyrate and TURSO can be present in the composition at a ratio by weight of between about 1:1 to about 4:1 (e.g., about 2:1 or about 3:1). In some embodiments, the ratio between sodium phenylbutyrate and TURSO is about 3:1.

The compositions described herein can include any pharmaceutically acceptable carrier, adjuvant, and/or vehicle. The term “pharmaceutically acceptable carrier or adjuvant” refers to a carrier or adjuvant that may be administered to a patient, together with a compound disclosed herein, and which does not destroy the pharmacological activity thereof and is nontoxic when administered in doses sufficient to deliver a therapeutic amount of the compound. As used herein the language “pharmaceutically acceptable carrier” includes saline, solvents, dispersion media, coatings, antibacterial and antifungal agents, isotonic and absorption delaying agents, and the like, compatible with pharmaceutical administration. The pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

Compositions of the present disclosure can include about 8% to about 24% w/w of dextrates (e.g., about 9% to about 23%, about 10% to about 22%, about 10% to about 20%, about 11% to about 21%, about 12% to about 20%, about 13% to about 19%, about 14% to about 18%, about 14% to about 17%, about 15% to about 16%, or about 15.6% w/w of dextrates). Both anhydrous and hydrated dextrates are contemplated herein. The dextrates of the present disclosure can include a mixture of saccharides developed from controlled enzymatic hydrolysis of starch. Some embodiments of any of the compositions described herein include hydrated dextrates (e.g., NF grade, obtained from JRS Pharma, Colonial Scientific, or Quadra).

Compositions of the present disclosure can include about 1% to about 6% w/w of sugar alcohol (e.g., about 2% to about 5%, about 3% to about 4%, or about 3.9% w/w of sugar alcohol). Sugar alcohols can be derived from sugars and contain one hydroxyl group (—OH) attached to each carbon atom. Both disaccharides and monosaccharides can form sugar alcohols. Sugar alcohols can be natural or produced by hydrogenation of sugars. Exemplary sugar alcohols include but are not limited to, sorbitol, xylitol, and mannitol. In some embodiments, the composition comprises about 1% to about 6% w/w (e.g., about 2% to about 5%, about 3% to about 4%, or about 3.9% w/w) of sorbitol.

Compositions of the present disclosure can include about 22% to about 35% w/w of maltodextrin (e.g., about 22% to about 33%, about 24% to about 31%, about 25% to about 32%, about 26% to about 30%, or about 28% to about 29% w/w, e.g., about 28.3% w/w of maltodextrin). Maltodextrin can form a flexible helix enabling the entrapment of the active ingredients (e.g., any of the phenylbutyrate compounds and bile acids described herein) when solubilized into solution, thereby masking the taste of the active ingredients. Maltodextrin produced from any suitable sources are contemplated herein, including but not limited to, pea, rice, tapioca, corn, and potato. In some embodiments, the maltodextrin is pea maltodextrin. In some embodiments, the composition includes about 28.3% w/w of pea maltodextrin. For example, pea maltodextrin obtained from Roquette (KLEPTOSE® LINECAPS) can be used.

The compositions described herein can further include sugar substitutes (e.g., sucralose). For example, the compositions can include about 0.5% to about 5% w/w of sucralose (e.g., about 1% to about 4%, about 1% to about 3%, or about 1% to about 2%, e.g., about 1.9% w/w of sucralose). Other sugar substitutes contemplated herein include but are not limited to aspartame, neotame, acesulfame potassium, saccharin, and advantame.

In some embodiments, the compositions include one or more flavorants. The compositions can include about 2% to about 15% w/w of flavorants (e.g., about 3% to about 13%, about 3% to about 12%, about 4% to about 9%, about 5% to about 10%, or about 5% to about 8%, e.g., about 7.3% w/w). Flavorants can include substances that give another substance flavor, or alter the characteristics of a composition by affecting its taste. Flavorants can be used to mask unpleasant tastes without affecting physical and chemical stability, and can be selected based on the taste of the drug to be incorporated. Suitable flavorants include but are not limited to natural flavoring substances, artificial flavoring substances, and imitation flavors. Blends of flavorants can also be used. For example, the compositions described herein can include two or more (e.g., two, three, four, five or more) flavorants. Flavorants can be soluble and stable in water. Selection of suitable flavorants can be based on taste testing. For example, multiple different flavorants can be added to a composition separately, which are subjected to taste testing. Exemplary flavorants include any fruit flavor powder (e.g., peach, strawberry, mango, orange, apple, grape, raspberry, cherry or mixed berry flavor powder). The compositions described herein can include about 0.5% to about 1.5% w/w (e.g., about 1% w/w) of a mixed berry flavor powder and/or about 5% to about 7% w/w (e.g., about 6.3% w/w) of a masking flavor. Suitable masking flavors can be obtained from e.g., Firmenich.

The compositions described herein can further include silicon dioxide (or silica). Addition of silica to the composition can prevent or reduce agglomeration of the components of the composition. Silica can serve as an anti-caking agent, adsorbent, disintegrant, or glidant. In some embodiments, the compositions described herein include about 0.1% to about 2% w/w of porous silica (e.g., about 0.3% to about 1.5%, about 0.5% to about 1.2%, or about 0.8% to about 1%, e.g., 0.9% w/w). Porous silica may have a higher H₂O absorption capacity and/or a higher porosity as compared to fumed silica, at a relative humidity of about 20% or higher (e.g., about 25%, 30%, 35%, 40%, 45%, 50%, 55%, 60%, 65%, 70%, 75%, 80%, 85%, 90%, or 95% or higher). The porous silica can have an H₂O absorption capacity of about 5% to about 40% (e.g., about 20% to about 40%, or about 30% to about 40%) by weight at a relative humidity of about 50%. The porous silica can have a higher porosity at a relative humidity of about 20% or higher (e.g., about 30%, 40%, 50%, 60%, 70%, 80%, 90% or higher) as compared to that of fumed silica. In some embodiments, the porous silica have an average particle size of about 2 μm to about 10 μm (e.g., about 3 μm to about 9 μm, about 4 μm to about 8 μm, about 5 μm to about 8 μm, or about 7.5 μm). In some embodiments, the porous silica have an average pore volume of about 0.1 cc/gm to about 2.0 cc/gm (e.g., about 0.1 cc/gm to about 1.5 cc/gm, about 0.1 cc/gm to about 1 cc/gm, about 0.2 cc/gm to about 0.8 cc/gm, about 0.3 cc/gm to about 0.6 cc/gm, or about 0.4 cc/gm). In some embodiments, the porous silica have a bulk density of about 50 g/L to about 700 g/L (e.g., about 100 g/L to about 600 g/L, about 200 g/L to about 600 g/L, about 400 g/L to about 600 g/L, about 500 g/L to about 600 g/L, about 540 g/L to about 580 g/L, or about 560 g/L). In some embodiments, the compositions described herein include about 0.05% to about 2% w/w (e.g., any subranges of this range described herein) of Syloid® 63FP (WR Grace).

The compositions described herein can further include one or more buffering agents. For example, the compositions can include about 0.5% to about 5% w/w of buffering agents (e.g., about 1% to about 4% w/w, about 1.5% to about 3.5% w/w, or about 2% to about 3% w/w, e.g., about 2.7% w/w of buffering agents). Buffering agents can include weak acid or base that maintain the acidity or pH of a composition near a chosen value after addition of another acid or base. Suitable buffering agents are known in the art. In some embodiments, the buffering agent in the composition provided herein is a phosphate, such as a sodium phosphate (e.g., sodium phosphate dibasic anhydrous). For example, the composition can include about 2.7% w/w of sodium phosphate dibasic.

The compositions can also include one or more lubricants. For example, the compositions can include about 0.05% to about 1% w/w of lubricants (e.g., about 0.10% to about 0.9%, about 0.2% to about 0.8%, about 0.3% to about 0.7%, or about 0.4% to about 0.6%, e.g., about 0.5% w/w of lubricants). Exemplary lubricants include, but are not limited to sodium stearyl fumarate, magnesium stearate, stearic acid, metallic stearates, talc, waxes and glycerides with high melting temperatures, colloidal silica, polyethylene glycols, alkyl sulphates, glyceryl behenate, and hydrogenated oil. Additional lubricants are known in the art. In some embodiments, the composition includes about 0.05% to about 1% w/w (e.g., any of the subranges of this range described herein) of sodium stearyl fumarate. For example, the composition can include about 0.5% w/w of sodium stearyl fumarate.

In some embodiments, the composition include about 29.2% w/w of sodium phenylbutyrate, about 9.7% w/w of TURSO, about 15.6% w/w of dextrates, about 3.9% w/w of sorbitol, about 1.9% w/w of sucralose, about 28.3% w/w of maltodextrin, about 7.3% w/w of flavorants, about 0.9% w/w of silicon dioxide, about 2.7% w/w of sodium phosphate (e.g., sodium phosphate dibasic), and about 0.5% w/w of sodium stearyl fumerate.

The composition can include about 3000 mg of sodium phenylbutyrate, about 1000 mg of TURSO, about 1600 mg of dextrates, about 400 mg of sorbitol, about 200 mg of sucralose, about 97.2 mg of silicon dioxide, about 2916 mg of maltodextrin, about 746 mg of flavorants (e.g., about 102 mg of mixed berry flavor and about 644 mg of masking flavor), about 280 mg of sodium phosphate (e.g., sodium phosphate dibasic), and about 48.6 mg of sodium stearyl fumerate.

Additional suitable sweeteners or taste masking agents can also be included in the compositions, such as but not limited to, xylose, ribose, glucose, mannose, galactose, fructose, dextrose, sucrose, maltose, steviol glycosides, partially hydrolyzed starch, and corn syrup solid. Water soluble artificial sweeteners are contemplated herein, such as the soluble saccharin salts (e.g., sodium or calcium saccharin salts), cyclamate salts, acesulfam potassium (acesulfame K), and the free acid form of saccharin and aspartame based sweeteners such as L-aspartyl-phenylalanine methyl ester, Alitame® or Neotame®. The amount of sweetener or taste masking agents can vary with the desired amount of sweeteners or taste masking agents selected for a particular final composition.

Pharmaceutically acceptable binders in addition to those described above are also contemplated. Examples include cellulose derivatives including microcrystalline cellulose, low-substituted hydroxypropyl cellulose (e.g., LH 22, LH 21, LH 20, LH 32, LH 31, LH30); starches, including potato starch; croscarmellose sodium (i.e., cross-linked carboxymethylcellulose sodium salt; e.g., Ac-Di-Sol®); alginic acid or alginates; insoluble polyvinylpyrrolidone (e.g., Polyvidon® CL, Polyvidon® CL-M, Kollidon® CL, Polyplasdone® XL, Polyplasdone® XL-10); and sodium carboxymethyl starch (e.g., Primogel® and Explotab®).

Additional fillers, diluents or binders may be incorporated such as polyols, sucrose, sorbitol, mannitol, Erythritol®, Tagatose®, lactose (e.g., spray-dried lactose, α-lactose, β-lactose, Tabletose®, various grades of Pharmatose®, Microtose or Fast-Floc®), microcrystalline cellulose (e.g., various grades of Avicel®, such as Avicel® PH101, Avicel® PH102 or Avicel® PH105, Elcema® P100, Emcocel®, Vivacel®, Ming Tai® and Solka-Floc®), hydroxypropylcellulose, L-hydroxypropylcellulose (low-substituted) (e.g., L-HPC-CH31, L-HPC-LH11, LH 22, LH 21, LH 20, LH 32, LH 31, LH30), dextrins, maltodextrins (e.g., Lodex® 5 and Lodex® 10), starches or modified starches (including potato starch, maize starch and rice starch), sodium chloride, sodium phosphate, calcium sulfate, and calcium carbonate.

The compositions described herein can be formulated or adapted for administration to a subject via any route (e.g., any route approved by the Food and Drug Administration (FDA)). Exemplary methods are described in the FDA's CDER Data Standards Manual, version number 004 (which is available at fda.give/cder/dsm/DRG/drg00301.html).

Pharmaceutical compositions are typically formulated to be compatible with its intended route of administration. Examples of routes of administration include parenteral (subcutaneous, intracutaneous, intravenous, intradermal, intramuscular, intra-articular, intraarterial, intrasynovial, intrasternal, intrathecal, intralesional and intracranial injection or infusion techniques), oral (e.g., inhalation or through a feeding tube), transdermal (topical), transmucosal, and rectal administration.

Pharmaceutical compositions can be in the form of a solution or powder for inhalation and/or nasal administration. In some embodiments, the pharmaceutical composition is formulated as a powder filled sachet. Suitable powders may include those that are substantially soluble in water. Pharmaceutical compositions may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions can be orally administered in any orally acceptable dosage form including, but not limited to, powders, capsules, tablets, emulsions and aqueous suspensions, dispersions and solutions. In the case of powders for oral administration, the powders can be substantially dissolved in water prior to administration. In the case of tablets for oral use, carriers which are commonly used include lactose and corn starch. Lubricating agents, such as magnesium stearate, may be added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Alternatively or in addition, the compositions can be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In some embodiments, therapeutic compositions disclosed herein can be formulated for sale in the US, imported into the US, and/or exported from the US. The pharmaceutical compositions can be included in a container, pack, or dispenser together with instructions for administration. In some embodiments, the invention provides kits that include the bile acid and phenylbutyrate compounds. The kit may also include instructions for the physician and/or patient, syringes, needles, box, bottles, vials, etc.

### Additional Therapeutic Agents and Further Combination Treatments

Any of the pharmaceutical compositions or methods described herein can further include one or more additional therapeutic agents in amounts effective for treating or achieving a modulation of at least one symptom of PSP. These therapies include therapies approved for treating progressive supranuclear palsy and those in development for treating progressive supranuclear palsy. These therapies include, but are not limited to, medications to treat movement disorders, medications to treat psychiatric disorders, psychotherapy, speech therapy, physical therapy, and occupational therapy. Medications to treat movement disorders include, but are not limited to, tetrabenazine, antipsychotic drugs, such as haloperidol, chlorpromazine, risperidone, and quetiapine, and other medications such as amantadine, levetiracetam, and, clonazepam. Medications to treat psychiatric disorders include, but are not limited to, antidepressants such as citalopram, fluoxetine, and sertraline, antipsychotic drugs such as quetiapine, risperidone, and olanzapine, and mood-stabilizing drugs, including anticonvulsants, such as valproate, carbamazepine, and lamotrigine. Psychotherapy includes, but is not limited to, talk therapy to help a subject manage behavioral problems, depression, and suicidal thoughts. Speech therapy includes, but is not limited to, improving a subjects ability to speak clearly, and improve function and control of muscles used for eating and swallowing. Physical therapy includes, but is not limited to, enhancing strength, flexibility, balance and coordination, reducing the risk of falls, and improve posture to lessen the severity of movement problems. Occupational therapy includes, but is not limited to, use of assistive devices that improve functional abilities such as handrails, and eating and drinking utensils for subjects with diminished motor skills.

The methods of the present disclosure can include administering to a subject one or more additional therapeutic agents (e.g., any of the additional therapeutic agents disclosed herein or known in the art), in combination with a bile acid (e.g., any of the suitable bile acids described herein) or a pharmaceutically acceptable salt thereof and a phenylbutyrate compound (e.g., any of the suitable phenylbutyrate compounds described herein). The additional therapeutic agent(s) can be administered for a period of time before administering the initial dose of a composition comprising a bile acid or a pharmaceutically acceptable salt thereof (e.g., TURSO) and a phenylbutyrate compound (e.g., sodium phenylbutyrate), and/or for a period of time after administering the final dose of the composition.

The combination of a bile acid or a pharmaceutically acceptable salt thereof, a phenylbutyrate compound, and one or more additional therapeutic agents can have a synergistic effect in treating a neurodegenerative disease (e.g., PSP). Smaller doses of the additional therapeutic agents may be required to obtain the same pharmacological effect, when administered in combination with a bile acid or a pharmaceutically acceptable salt thereof, and a phenylbutyrate compound. In some embodiments, the amount of the additional therapeutic agent(s) administered in combination with a bile acid or a pharmaceutically acceptable salt thereof and a phenylbutyrate compound can be reduced by at least about 10% (e.g., at least about 15%, 20%, 25%, 30%, 35%, 40%, 45%, 50%, or 55%) compared to the dosage amount used when the additional therapeutic agent(s) is administered alone. Additionally, or alternatively, the methods of the present disclosure can reduce the required frequency of administration of other therapeutic agents (e.g., other PSP therapeutic agents) to obtain the same pharmacological effect.

Some embodiments of the present disclosure provide a method of treating at least one symptom of PSP or preventing the onset of PSP in a human subject, the method comprising administering to the human subject an effective amount of (a) a bile acid or a pharmaceutically acceptable salt thereof (e.g., any of the bile acid or a pharmaceutically acceptable salt thereof described herein); (b) a phenylbutyrate compound (e.g., any of the phenylbutyrate compounds described herein); and (c) one of the additional therapeutic PSP agents listed above, to thereby treat at least one symptom of PSP or prevent the onset of PSP in the human subject.

The bile acid or a pharmaceutically acceptable salt thereof and the phenylbutyrate compound can be administered shortly after a meal (e.g., within two hours of a meal) or under fasting conditions. The subject may have consumed food items (e.g., solid foods or liquid foods) less than 2 hours before administration of a bile acid or a pharmaceutically acceptable salt thereof and/or a phenylbutyrate compound; or will consume food items less than 2 hours after administration of one or both of the compounds. Food items may affect the rate and extent of absorption of the bile acid or a pharmaceutically acceptable salt thereof and/or the phenylbutyrate compound. For instance, food can change the bioavailability of the compounds by delaying gastric emptying, stimulating bile flow, changing gastrointestinal pH, increasing splanchnic blood flow, changing luminal metabolism of the substance, or physically or chemically interacting with a dosage form or the substance. The nutrient and caloric contents of the meal, the meal volume, and the meal temperature can cause physiological changes in the GI tract in a way that affects drug transit time, luminal dissolution, drug permeability, and systemic availability. In general, meals that are high in total calories and fat content are more likely to affect the GI physiology and thereby result in a larger effect on the bioavailability of a drug. The methods provided herein can further include administering to the subject a plurality of food items, for example, less than 2 hours (e.g., less than 1.5 hour, 1 hour, or 0.5 hour) before or after administering the bile acid or a pharmaceutically acceptable salt thereof, and/or the phenylbutyrate compound.

### EXAMPLES

Additional embodiments are disclosed in further detail in the following examples, which are provided by way of illustration and are not in any way intended to limit the scope of this disclosure or the claims.

### Example 1: Evaluation of the Safety, Tolerability, Efficacy and Activity of AMX0035, a Fixed Combination of Phenylbutyrate (PB) and Tauroursodeoxycholic Acid (TUDCA), for Treatment of PSP

### Dose Rationale for AMX0035

PSP is a tauopathy characterized by degeneration in the cerebral cortex, basal ganglia, and brainstem. In AMX8000 (PEGASUS), a clinical study conducted by the Sponsor in participants with AD, AMX0035 administered at doses of 1 sachet BID demonstrated a significant decrease in tau proteins. Therefore, it is hypothesized that AMX0035 administered at doses of 1 sachet BID may mitigate symptoms of PSP and slow the progression caused by the neurodegenerative processes.

The dose of AMX0035 selected for this study consists of 3 g PB and 1 g TURSO per sachet. In the previously completed and currently ongoing clinical studies, AMX0035 has been generally well-tolerated up to a dose of 3 g PB and 1 g TURSO administered BID.

### Pharmacokinetics of PB and TURSO Following Oral Administration of Single and Multiple Doses of AMX0035

The pharmacokinetics (PK) of PB and TURSO after a single dose administration of AMX0035 were determined in adult (40 to 55 years of age) male and female normal healthy participants. Fourteen participants received a single dose of AMX0035 on two occasions (with a washout between periods of no less than 4 days), after fasting or after a high-fat standardized meal in a balanced, two-period, cross-over design (Study A35-002).

PB is rapidly absorbed and reaches maximum plasma concentrations (Cₘₐₓ) by a median time of 1 hour. Clearance is rapid, and the terminal half-life (t_{1/2}) is less than 1 hour. Administration of AMX0035 in the presence of a high-fat meal resulted in both significantly slower absorption and lower overall exposure of PB, although the terminal half-life of PB remained relatively fast (approximately 0.5 hours) under both the fed and fasted condition. The primary metabolite of PB is phenylacetate (PAA), which occurs endogenously as a phenylalanine metabolite. TURSO, whether given with or without food, is slowly absorbed. Many participants had two or three peaks of absorption, which is consistent with bile acid storage and release upon a meal/snack. Administration of AMX0035 in the presence of a high-fat meal resulted in similar Cₘₐₓ and a slightly (<1-fold) higher increase in exposure to TURSO as measured by area under the concentration-time curve from time 0 to the last measurable timepoint (AUC₀₋ₗₐₛₜ). The terminal t_{1/2} of TURSO was slow under both the fed and fasting conditions (average 3.6 to 4.8 hours, respectively). The primary metabolites of TURSO include ursodeoxycholic acid (UDCA) and glycoursodeoxycholic acid (GUDCA). All three are naturally occurring hydrophilic bile acids and subject to extensive enterohepatic recirculation.

An additional PK study, Study A35-007, was a Phase 1, open label study designed to determine the plasma levels of PB and TURSO and their metabolites following administration of a single dose and multiple doses of AMX0035 in healthy Japanese and Caucasian male and female participants. On Day 1 of the study, participants were dosed with a single dose of AMX0035 followed by a 24-hour blood sample collection to assess single dose PK of AMX0035. Thereafter, the participants were dosed twice a day with AMX0035 from Days 2 through 6, and another 24-hour blood sample collection was performed following the morning dose on Day 7 to assess the steady-state PK of AMX0035. Results from the study show that AMX0035 was well-tolerated following multiple doses, and safety profiles were similar between the Caucasian and Japanese participants. The study is clinically complete and data analysis is ongoing.

### Study Justification

The decision to conduct this study in two phases—specifically, with the Phase 2b portion to be completed before a determination is made whether to proceed to Phase 3—reflects two key aims. The first is to reduce to the greatest possible extent the burden placed upon participants living with PSP—a debilitating, chronic disease that impacts multiple facets of day-to-day life—and their caregivers. The second is to reassure of the potential efficacy and acceptable safety profile of AMX0035 in this patient population.

The double-blind phases in the Phase 2b and Phase 3 portions of this study are intended to determine the efficacy and safety/tolerability of AMX0035 in people living with PSP. Participants will be randomized in a 3:2 ratio to receive double-blind AMX0035 or matching placebo, respectively, for up to 52 weeks before they may, if still eligible, consent to roll over into an optional OLE phase. A treatment duration of up to 52 weeks, and comparison with a placebo control, are considered adequate and appropriate to evaluate the efficacy of AMX0035 on the primary and secondary study endpoints, given the natural history of PSP and the lack of any approved therapies for the disease.

The OLE phases in the Phase 2b and Phase 3 portions of this study are intended to further characterize the long-term efficacy and safety/tolerability of AMX0035.

### Potential Risks and Benefits

AMX0035 was administered in multiple clinical studies and was generally well tolerated. The main AEs reported to date have been mild and transient in nature and were related to gastrointestinal (GI) abnormalities (e.g., nausea and diarrhea) and were not clinically concerning. GI AEs generally subsided after 2 to 3 weeks of continued AMX0035 administration.

Evidence of efficacy demonstrated in nonclinical studies and safety data from clinical studies support the assessment of safety and efficacy in the treatment of PSP with AMX0035.

The participants in this study with PSP may benefit from treatment with AMX0035. Additionally, the results from this study may be used to develop therapies for patients with other neurodegenerative diseases.

Based on the current available information on pathogenesis of PSP, mechanism of action of AMX0035 and the known safety profile of AMX0035, the potential benefits of treatment with AMX0035 outweigh the potential risks in this clinical study.

STUDY OBJECTIVES AND ENDPOINTS

| | |
|---|---|
| Objective | Endpoint |

Primary

| | |
|---|---|
| To assess the impact of AMX0035 | Change from Baseline at Week 52 in |
| compared to placebo on disease | the PSPRS Score* |
| progression rate as measured by the | |
| Progressive Supranuclear Palsy (PSP) | |
| Rating Scale (PSPRS) | |

Secondary

| | |
|---|---|
| To assess the impact of AMX0035 | Change from Baseline at Week 52 in the |
| compared to placebo on disease | PSPRS Score* |
| progression rate as measured by the | |
| Progressive Supranuclear Palsy (PSP) | |
| Rating Scale (PSPRS) | |
| To evaluate the efficacy of AMX0035 | Change from Baseline at Week 52 in the |
| compared to placebo on motor aspects of | MDS-UPDRS Part II Score |
| activities of daily living as measured on the | |
| Movement Disorder Society-Unified | |
| Parkinson's Disease Rating (MDS- | |
| UPDRS) Scale Part II | |
| To evaluate the safety and tolerability of | Frequency of treatment-emergent |
| AMX0035 in participants with PSP | adverse events (TEAEs) and serious |
| | adverse events (SAEs) |

Exploratory

| | |
|---|---|
| To evaluate the effect of AMX0035 | Change from Baseline at Week 52 in |
| compared to placebo on activities of daily | SEADL score |
| living (ADL) as measured by the Schwab | |
| and England ADL (SEADL) scale | |
| To evaluate the effect of AMX0035 | Change from Baseline at Week 52 in |
| compared to placebo on Global Impression | CGI-C score |
| of Change (CGI-C) | |
| To evaluate the effect of AMX0035 | Change from Baseline at Week 52 in |
| compared to placebo on brain atrophy of | brain volume as measured by MRI |
| selected structures as measured by | (whole brain and volumes of third |
| volumetric magnetic resonance imaging | ventricle, midbrain, and frontal lobe as |
| (MRI) | combined imaging read-out) |
| To evaluate the effect of AMX0035 | Change from Baseline at Week 52 in |
| compared to placebo on Global Impression | Global Impression of Severity (CGI S) |
| of Severity (CGI S) | |
| To evaluate the effect of AMX0035 | Change from Baseline at Week 52 in |
| compared to placebo on Montreal | MoCA Score |
| Cognitive Assessment (MoCA) Score | |
| To evaluate the effect of AMX0035 | Change from Baseline at Week 52 in |
| compared to placebo on fluid biomarkers | fluid biomarkers of neuronal injury and |
| of neuronal injury and neuro inflammation | neuro inflammation |
| To evaluate the effect of AMX0035 | Change from Baseline at Week 52 in |
| compared to placebo on caregiver burden | caregiver burden measured by the Zarit |
| measured by the Zarit Burden Interview | Burden Interview (ZBI) |
| (ZBI) | |
| To evaluate the effect of AMX0035 | Change from Baseline at Week 52 in |
| compared to placebo on quality of life as | quality of life as measured by the |
| measured by the EQ5D-5L | EQ5D-5L |
| To evaluate the effect of AMX0035 | Change from Baseline at Week 52 in |
| compared to placebo on medical resource | medical resource utilization parameters |
| utilization | |
| To evaluate the effect of AMX0035 | Overall Survival |
| compared to placebo on Overall Survival | |
| from randomization to death | |

*A primary endpoint-which meets evidentiary requirements of the United States of America (USA) and outside of the USA, respectively-was selected as follows: change from baseline in the 10-item PSPRS at Week 52 (for the USA) and change from baseline in the 28-item PSPRS at Week 52 (for outside of the USA). For each region, only the endpoint which meets evidentiary requirements of that region is considered the primary endpoint; the other is considered a secondary endpoint, as detailed in the study statistical analysis plan.

### Study Design

A35-009 (ORION) is a blinded, randomized, placebo-controlled, two-part Phase 2b/3 study intended to evaluate the efficacy and safety of AMX0035 in participants with PSP.

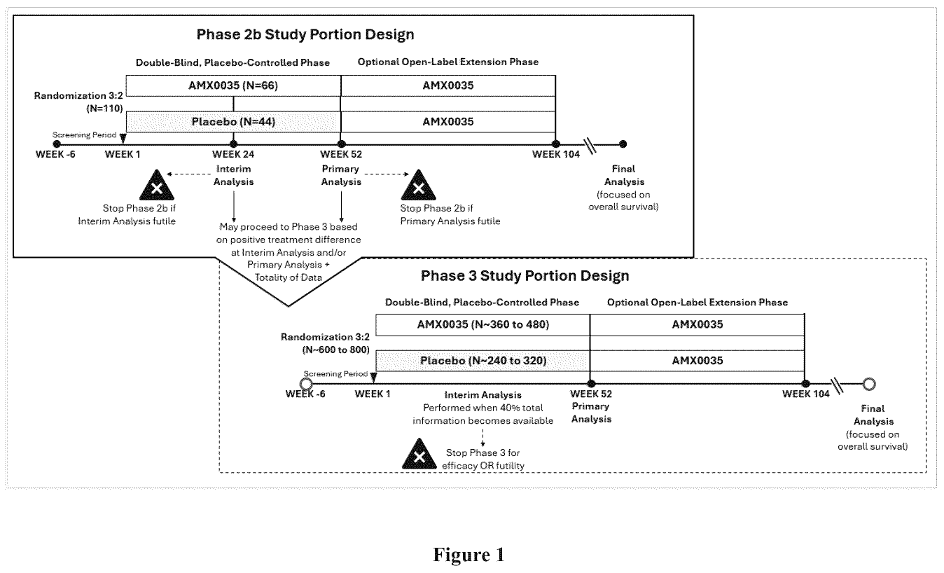

The design of the overall study is presented in FIG. 1. The Phase 2b and Phase 3 study portions are planned to feature an identical design: a randomized, double-blind, placebo-controlled phase that is followed by an optional open-label extension (OLE) phase.

The procedures to be performed in both study portions, along with their corresponding timing, are summarized in the Schedules of Activities for the double-blind (Table 1) and OLE (Table 2) phases.

If the study is not stopped for futility at the time of the Week 24 IA, the Phase 2b primary analysis will be performed once all of the participants randomized in that portion have completed 52 weeks of study treatment, have withdrawn from study treatment, or have died.

All study participants will continue to be followed for their survival status. Overall survival will be the focus of the final analysis, as detailed below in Section “Interim, Primary, and Final Analyses”.

TABLE 1
Schedule of Activities for A35-009 (ORION) Double-blind Phases
- Phase 2b and Phase 3 (if conducted) Study Portions

| | Period: | | | | | | | | |
|---|---|---|---|---|---|---|---|---|---|
| | Screening | Baseline ^{t} | Double-blind Treatment Period | | | | | | |
| | Visit: | | | | | | | | |
| | | Day | Week | Week | Week | Week | Weeks | Week | Weeks |
| | Screening | 1 | 2 ^{a} | 4 | 8 | 12 | 16, 20 | 24 | 28, 32 |
| Window: | up to | up to | up to | ±4 | ±4 | ±7 | ±4 | ±7 | ±4 |
| | 6 weeks | 5 days | +7 | days | days | days | days | days | days |
| | prior to | prior to | days | | | | | | |
| | first dose | first dose ^{r} | | | | | | | |
| Clinic Visit: | X | X | X (optional) ^{a} | X | | X | | X | |
| Activities | | | | | | | | | |
| Written informed | X | | | | | | | | |
| consent (participant | | | | | | | | | |
| and study partner) | | | | | | | | | |
| Inclusion/exclusion | X | X | | | | | | | |
| criteria | | | | | | | | | |
| Medical history | X | X ^{c} | | | | | | | |
| (including PSP | | | | | | | | | |
| diagnosis/history/ | | | | | | | | | |
| demographics) ^{c} | | | | | | | | | |
| Physical examination/ | X | X | | X | | X | | X | |
| Medical resource | | | | | | | | | |
| utilization review | | | | | | | | | |
| Vital signs ^{d} | X | X | | X | | X | | X | |
| Body weight and | X | X | | X | | X | | X | |
| height ^{e} | | | | | | | | | |
| Neurological | X | X | | | | | | X | |
| examination | | | | | | | | | |
| Urine screen for | X | | | | | | | | |
| drugs of abuse | | | | | | | | | |
| Pregnancy test ^{f} | X | X | | X | X | X | X | X | X |
| 12-lead ECG ^{g} | | X | | X | | X | | X | |
| Safety laboratory | X | X | | X | | X | | | |
| assessments | | | | | | | | | |
| (hematology, clinical | | | | | | | | | |
| chemistry, urinalysis, | | | | | | | | | |
| coagulation) ^{h} | | | | | | | | | |
| Brain MRI ⁱ | X | | | | | | | X | |
| Lumbar puncture ^{j} | | X | | | | | | | |
| Blood sampling for | | X | | X | | X | | X | |
| biomarkers ^{k} | | | | | | | | | |
| Blood sampling for PK | | X | | | | | | X | |
| (first 110 participants | | | | | | | | | |
| randomized in study) ^{l} | | | | | | | | | |
| Randomization | | X | | | | | | | |
| MMSE ^{m, o} | X | | | | | | | | |
| PSPRS (28-item) ^{n, o} | X | X | | X | | X | | X | |
| PSPRS (10-item) ^{n, o} | | X | | X | | X | | X | |
| MDS-UPDRS Part II ^{o} | X | X | | X | | X | | X | |
| SE-ADL ^{o} | | X | | | | X | | X | |
| MoCA test | | X | | | | | | X | |
| CGI-C and CGI-S ^{o} | | X ^{s} | | | | X | | X | |
| EQ-5D-5L ^{o} | | X | | | | | | X | |
| C-SSRS ^{o, p} | X | X | | | | X | | X | |
| ZBI ^{q} | | X | | | | | | X | |
| Review use of | X | X | X | X | X | X | X | X | X |
| prior/concomitant | | | | | | | | | |
| medications | | | | | | | | | |
| Adverse event | X ^{f} | X | X | X | X | X | X | X | X |
| assessment | | | | | | | | | |
| Study drug | | | X | X | | X | | X | |
| accountability/ | | | | | | | | | |
| compliance | | | | | | | | | |

| | |
|---|---|
| Dispense study drug ^{g} | X |

| | Period: | | | |
|---|---|---|---|---|
| | | | | Safety |
| | Double-blind Treatment Period | | | F/U |
| | Visit: | | | |
| | Week | Weeks | Week 52 | Week |
| | 36 | 40, 44, 48 | or EOT ^{b} | 56 |
| Window: | ±7 | ±4 | ±7 | ±7 |
| | days | days | days | days |
| Clinic Visit: | X | | X | |
| Activities | | | | |
| Written informed | | | | |
| consent (participant | | | | |
| and study partner) | | | | |
| Inclusion/exclusion | | | | |
| criteria | | | | |
| Medical history | | | | |
| (including PSP | | | | |
| diagnosis/history/ | | | | |
| demographics) ^{c} | | | | |
| Physical examination/ | X | | X | |
| Medical resource | | | | |
| utilization review | | | | |
| Vital signs ^{d} | | | X | |
| Body weight and | X | | X | |
| height ^{e} | | | | |
| Neurological | X | | X | |
| examination | | | | |
| Urine screen for | | | | |
| drugs of abuse | | | | |
| Pregnancy test ^{f} | X | X | X | X |
| 12-lead ECG ^{g} | | | X | |
| Safety laboratory | X | | X | |
| assessments | | | | |
| (hematology, clinical | | | | |
| chemistry, urinalysis, | | | | |
| coagulation) ^{h} | | | | |
| Brain MRI ⁱ | | | X | |
| Lumbar puncture ^{j} | | | X | |
| Blood sampling for | X | | X | |
| biomarkers ^{k} | | | | |
| Blood sampling for PK | | | X | |
| (first 110 participants | | | | |
| randomized in study) ^{l} | | | | |
| Randomization | | | | |
| MMSE ^{m, o} | | | | |
| PSPRS (28-item) ^{n, o} | X | | X | |
| PSPRS (10-item) ^{n, o} | X | | X | |
| MDS-UPDRS Part II ^{o} | X | | X | |
| SE-ADL ^{o} | X | | X | |
| MoCA test | | | X | |
| CGI-C and CGI-S ^{o} | X | | X | |
| EQ-5D-5L ^{o} | | | X | |
| C-SSRS ^{o, p} | X | | X | |
| ZBI ^{q} | | | X | |
| Review use of | X | X | X | |
| prior/concomitant | | | | |
| medications | | | | |
| Adverse event | X | X | X | X |
| assessment | | | | |
| Study drug | X | | X | |
| accountability/ | | | | |
| compliance | | | | |

| | |
|---|---|
| Dispense study drug ^{g} | X |

^{a} After 2 weeks of once-daily dosing of study drug, participants to increase to 1 dose BID (taken morning and evening) following review at the Week 2 visit. The Week 2 visit can be conducted either at the clinic or via telephone or a telehealth virtual platform, based on the Investigator's assessment and the participant's agreement.
^{b} In the case of early termination, the EOT visit should include all Week 52 assessments. The Safety Follow-up visit will also occur via telephone 4 weeks (±7 days) after the participant's last study drug dose. Following completion of or discontinuation from study treatment, ongoing reporting of survival status will be recorded until time of death or until the end of study is announced (see Section “Start and End of Study Definitions”).
^{c} Medical history includes collection of demographics (demographic information will consist of participant date of birth [or age], sex, race, and ethnicity [where applicable]); assessment of PSP clinical features (refer to Section “PSP Clinical Features”]); and history of alcohol, drugs of abuse, and tobacco abuse. An updated medical history will be collected on Day 1 prior to randomization.
^{d} Vital signs will include temperature, heart rate, and blood pressure. The position of the participant (i.e., supine, semi-supine, orthostatic) during blood pressure and heart rate assessments is at the discretion of the Investigator, and these assessments must be preceded by 5 minutes of rest for the participant. Blood pressure and heart rate will be assessed using the arm opposite to that used for blood draws. For visits in which both vital signs and blood sample(s) are collected, vital signs should be obtained prior to any blood collection.
^{e} Height will be collected at Screening visit only.
^{f} A serum pregnancy test will be obtained from all female participants during Screening (within 7 days prior to the first dose of study drug), at Week 52 if the participant does not continue into the OLE phase of the study, and at an EOT Visit if the participant discontinues early from study treatment.
- FSH will be required at Screening for female participants in a menopausal or postmenopausal state only.
- Day 1 urine testing will be performed for WOCBP only, prior to dosing.
- Monthly urine pregnancy tests will be obtained for WOCBP only. Urine tests will be sent by the site to the participant and recorded during the remote follow-up visit phone call for the months between the in-clinic visits.
^{g} Two 12-lead ECGs are to be performed at the indicated visits after the participant has been supine for at least 5 minutes. All ECGs will be read and interpreted locally.
^{h} The coagulation assessment is only required prior to each study lumbar puncture and must be processed by the central laboratory. The determination of timing for the coagulation assessment relative to each lumbar puncture in the study is to be guided by institutional procedure and Investigator discretion.
ⁱ If sedation is required, it should occur after all scales and cognitive testing have been performed or, if this is not possible, at least 48 hours before all scales and cognitive testing are scheduled.
- The Screening MRI assessment will be performed locally and will be read locally by the Investigator.
- Scans at all protocol-required timepoints will be uploaded to the central imaging vendor.
- Scans that are of poor quality (e.g., due to participant motion during the scan, inadequate coverage of the brain, improper positioning of the head, use of incorrect scan or geometry parameters, or noisy image) as determined by the MRI technologist at the imaging center, the reviewing radiologist, or central imaging vendor will be repeated at the earliest possible time but within 15 days of the protocol required timepoint to obtain a scan of good quality. Refer to the study-specific Imaging Manual for additional information.
^{j} Lumbar puncture must be performed >3 months from previous lumbar puncture. Contraindications, including prohibited medications and coagulation, to lumbar puncture must be reviewed by the Investigator before the lumbar puncture.
^{k} Biomarker samples will be collected no more than 30 minutes prior to dosing on Day 1. Blood samples collected at Week 24 and Week 52 may be collected at any time during these visits.
^{l} PK blood samples will be collected from the first 110 randomized participants at 1, 4, and 6 hours postdose (±20 minutes). A PK sample will not be taken at an Early Termination visit if the participant discontinued from study treatment more than 48 hours prior to the visit.
^{m} MMSE is administered only during Screening to assess eligibility.
ⁿ The 28-item PSPRS should be obtained prior to the 10-item PSPRS. At Screening, only the 28-item PSPRS will be performed.
^{o} Scales should be prioritized ahead of other study procedures at each unique visit.
^{p} The C-SSRS “Lifetime/Recent” version will be administered at Screening. The C-SSRS “Since Last Visit” version will be administered at all subsequent clinic visits.
^{q} ZBI will be conducted, but there is no mandatory timing for this assessment in relation to the timing of the other study procedures conducted at each unique visit.
^{r} AEs will be recorded from Day 1 until 28 days (+7 days) after the participant's last dose of study drug (at Safety Follow-up is acceptable). SAEs will be recorded from the time of consent.
^{s} The first dose of study drug will be administered in the clinic after ALL Screening and baseline (Day 1 visit) procedures have been completed, with the exception of baseline PK (which is to be collected from the first 110 randomized participants and must be obtained post-first dose). All participants will be trained on the preparation of study drug and frequency of dosing prior to leaving the clinic on Day 1. If at any point during the study, circumstances should arise that prevent participants from completing in-clinic visits, the Sponsor may implement direct-to-participant or direct-from-participant study drug shipment via a Sponsor-approved courier service or via site courier service per institutional policies.
^{t} The Baseline visit procedures may begin up to 5 days before the first dose of study drug, and may be conducted over multiple days, as needed. All Baseline visit procedures, including randomization and the first dose of study drug, must occur within the 6-week Screening period.
^{u} Only the CGI-S (i.e., not the CGI-C) is required at the Baseline visit.
Abbreviations: AE = adverse event; BID = twice daily; CGI-C = Clinical Global Impression Scale - Change; CGI-S = Clinical Global Impression Scale -Severity; C-SSRS = Columbia-Suicide Severity Rating Scale; ECG = electrocardiogram; EOT = End of Treatment; EQ-5D-5L = EuroQOL 5 Dimension 5-Level; FSH = follicle-stimulating hormone; F/U = follow-up; MDS-UPDRS = Movement Disorder Society Unified Parkinson's Disease Rating Scale; MMSE = Mini Mental State Exam; MoCA = Montreal Cognitive Assessment; MRI = magnetic resonance imaging; OLE = open-label extension; PK = pharmacokinetics; PSP = progressive supranuclear palsy; PSPRS = Progressive Supranuclear Palsy Rating Scale; SAE = serious adverse event; SE-ADL = Schwab and England Activities of Daily Living; WOCBP = women of childbearing potential; ZBI = Zarit Burden Interview.

TABLE 2
Schedule of Activities for A35-009 (ORION) Open-label Extension
Phases - Phase 2b and Phase 3 (if conducted) Study Portions

| | Period: | | | | | | | |
|---|---|---|---|---|---|---|---|---|
| | OLE Phase | | | | | | | |
| | Baseline | Open-label Treatment Period | | | | | Safety F/U | |
| | Visit: | | | | | | | |
| | Week 52 of | | | | | | Week | |
| | Double-blind | Week | Week | Week | Week | Week | 104 or | Week |
| | Phase ^{a} | 54 | 56 | 64 | 76 | 88 | EOT ^{b} | 108 |
| Window: | ±7 | +7 | ±4 | ±4 | ±7 | ±4 | ±7 | +7 |
| | days | days | days | days | days | days | days | days |
| Clinic Visit: | X | X (optional) ^{a} | X | | X | | X | |
| Activities | | | | | | | | |
| Written informed consent | X | | | | | | | |
| (participant only) ^{c} | | | | | | | | |
| Physical examination/ | X | | X | | X | | X | |
| Medical resource | | | | | | | | |
| utilization review | | | | | | | | |
| Vital signs ^{d} | X | | X | | X | | X | |
| Body weight | X | | X | | X | | X | |
| Neurological examination | X | | | | X | | X | |
| Pregnancy test ^{e} | | | X | X | X | X | X | X |
| 12-lead ECG ^{f} | X | | | | | | | |
| Safety laboratory | X | | X | | X | | X | |
| assessments (hematology, | | | | | | | | |
| clinical chemistry, | | | | | | | | |
| urinalysis, coagulation) ^{g} | | | | | | | | |
| Brain MRI ^{b} | X | | | | | | X | |
| Lumbar puncture ⁱ | X | | | | | | optional | |
| Blood sampling for | X | | | | X | | X | |
| biomarkers | | | | | | | | |
| Blood sampling for PK | X | | | | | | | |
| (first 110 participants | | | | | | | | |
| randomized in study) | | | | | | | | |
| PSPRS (28-item) ^{j, k} | X | | | | X | | X | |
| PSPRS (10-item) ^{j, k} | X | | | | X | | X | |
| MDS-UPDRS Part II ^{k} | X | | | | X | | X | |
| SE-ADL ^{k} | X | | | | X | | X | |
| MoCA | X | | | | X | | X | |
| CGI-C and CGI-S ^{k} | X | | | | X | | X | |
| EQ-5D-5L ^{k} | X | | | | X | | X | |
| C-SSRS ^{k, l} | X | | | | X | | X | |
| ZBI ^{m} | X | | | | X | | X | |
| Review use of concomitant | X | X | X | X | X | X | X | |
| medications | | | | | | | | |
| Adverse event assessment | X | X | X | X | X | X | X | X |
| Study drug | X | X | X | X | X | X | X | |
| accountability/compliance | | | | | | | | |

| | |
|---|---|
| Dispense study drug ⁿ | X |

^{a} Assessments listed for the Week 52 visit will be from the Week 52 visit of the Treatment Period in the double-blind phase from which the participant is rolling over. These assessments do not need to be repeated.
^{b} In the case of early treatment termination, the EOT visit should include all Week 104 assessments. The Safety Follow-up visit will also occur via telephone 4 weeks (+7 days) after the participant's last study drug dose. Following completion of or discontinuation from study treatment, ongoing reporting of survival status will be recorded until the participant's time of death or until the end of the study is announced (see Section “Start and End of Study Definitions”).
^{c} Open-label extension phase ICF required.
^{d} Vital signs will include temperature, heart rate, and blood pressure. The position of the participant (i.e., supine, semi-supine, orthostatic) during blood pressure and heart rate assessments is at the discretion of the Investigator, and these assessments must be preceded by 5 minutes of rest for the participant. Blood pressure and heart rate will be assessed using the arm opposite to that used for blood draws. For visits in which both vital signs and blood sample(s) are collected, vital signs should be obtained prior to any blood collection.
^{e} A urine pregnancy test should be obtained as indicated for WOCBP only. Pregnancy tests will be sent by the site to the participant and captured through the remote follow-up visit phone call for the months between the in-clinic visits. A serum pregnancy test will be obtained from all female participants at the Week 104/EOT visit.
^{f} Two 12-lead ECGs will be performed at the indicated visits after the participant has been supine for at least 5 minutes. All ECGs will be read and interpreted locally.
^{g} The coagulation assessment is only required prior to each study lumbar puncture and must be processed by the central laboratory. The determination of timing for the coagulation assessment relative to each lumbar puncture in the study is to be guided by institutional procedure and Investigator discretion.
^{h} If sedation is required, it should occur after all scales and cognitive testing have been performed.
-Scans at all protocol-required timepoints will be uploaded to the central imaging vendor. Scans that are of poor quality (e.g., due to participant motion during the scan, inadequate coverage of the brain, improper positioning of the head, use of incorrect scan or geometry parameters, or noisy image) as determined by the MRI technologist at the imaging center, reviewing radiologist, or Sponsor will be repeated at the earliest possible time but within 15 days of the protocol required timepoint to obtain a scan of good quality.
-Refer to the study-specific imaging manual for additional information.
ⁱ Lumbar puncture must be performed >3 months from previous lumbar puncture. Contraindications, including prohibited medications and coagulation, to lumbar puncture must be reviewed by the Investigator before the lumbar puncture. Lumbar puncture is optional at Week 104/EOT.
^{j} The 28-item PSPRS should be obtained prior to the 10-item PSPRS.
^{k} Scales should be prioritized ahead of other study procedures at each unique visit.
^{l} The C-SSRS “Since Last Visit” version will be administered at all clinic visits.
^{m} ZBI will be conducted, but there is no mandatory timing for this assessment in relation to the timing of the other study procedures conducted at each unique visit.
ⁿ Participants to increase to 1 dose BID (taken morning and evening) after review at the Week 54 visit, which can be conducted either at the clinic or via telephone or a telehealth platform based on the Investigator's assessment and the participant's agreement.
If at any point during the study, circumstances should arise that prevent participants from completing in-clinic visits, the Sponsor may implement direct-to-participant or direct-from-participant study drug shipment via a Sponsor-approved courier service or via site courier service per institutional policies.
Abbreviations: AE = adverse event; BID = twice daily; CGI-C = Clinician-reported Global Impression of Change; C-SSRS = Columbia Suicide Severity Rating Scale; ECG = electrocardiogram; EOT = End of Treatment; EQ-5D-5L = EuroQoL 5 Dimension 5-Level; F/U = follow-up; ICF = informed consent form; MDS-UPDRS = Movement Disorder Society Unified Parkinson's Disease Rating Scale; MoCA = Montreal Cognitive Assessment; MRI = magnetic resonance imaging; PK = pharmacokinetics; PSP = progressive supranuclear palsy; PSPRS = Progressive Supranuclear Palsy Rating Scale; SAE = serious adverse event; SE-ADL = Schwab and England Activities of Daily Living; WOCBP = women of childbearing potential; ZBI = Zarit Burden Interview.

### Phase 2b Study Portion

### Phase 2b Double-Blind Phase

The Phase 2b double-blind phase includes a Screening Period of up to 6 weeks, a double-blind Treatment Period of up to 52 weeks, and a remote Safety Follow-up visit occurring 4 weeks (+7 days) after the End-of-Treatment (EOT) visit.

Approximately 110 participants in the double-blind phase are planned to be randomized in a 3:2 ratio to receive either AMX0035 or matching placebo. Participants will receive their first dose of study drug in the clinic. They will be instructed to take 1 dose daily and then increase to 1 dose twice daily (BID; taken morning and evening) starting at the Week 2 visit. The Week 2 visit can be conducted either at the clinic or via telephone or a telehealth virtual platform, based on the Investigator's assessment and the participant's agreement.

Participants will return to the clinic for assessments at Week 4, Week 12, Week 24, Week 36, and Week 52. The Week 52 visit will serve as the baseline visit for those participants who remain eligible and consent to continue into the Phase 2b OLE phase, as described immediately below.

### Phase 2b OLE Phase

To enter the Phase 2b OLE phase from the double-blind phase, participants must provide consent and continue not to meet any of the study discontinuation criteria (as per Section “Discontinuation from Study Treatment”) in the judgment of the Investigator.

The Treatment Period for the Phase 2b OLE phase will be up to 52 weeks in duration. Participants will receive their first dose of open-label AMX0035 in the clinic under the supervision of site personnel. They will be instructed to take 1 dose of open-label AMX0035 daily and then increase to 1 dose (BID; taken morning and evening) starting at the Week 54 visit. The Week 54 visit can be conducted either at the clinic or via telephone or a telehealth platform, based on the Investigator's assessment and the participant's agreement.

Participants who continue into the Phase 2b OLE phase will remain in the study for an additional 52 weeks in total, and will return to the clinic for assessments at Week 56, Week 76, and Week 104. Participants will then meet with study staff via telephone or a telehealth platform for the Safety Follow-up visit at Week 108.

### Phase 2b Interim Analysis

The enrollment of the Phase 2b study portion will be considered complete when all of the participants in that portion have been randomized. Once these participants have completed their Week 24 study procedures, a preplanned IA will be conducted.

As noted previously, depending on the results of the Week 24 IA, the study may stop for futility or continue as detailed immediately below and in Section “Interim, Primary, and Final Analyses”.

### Phase 2b Primary Analysis

If the study is not stopped for futility at the time of the Week 24 IA, the Phase 2b primary analysis will be performed once all of the participants randomized in that portion have completed 52 weeks of study treatment, have withdrawn from study treatment, or have died. Refer to Section “Interim, Primary, and Final Analyses” for additional information.

### Phase 2b Final Analysis

The final analysis for the Phase 2b study portion will be performed after that portion is considered complete. This analysis will focus on overall survival.

Criteria and Process for Determining whether to Initiate Phase 3 Study Portion

Should either the Phase 2b IA or (if performed) Phase 2b primary analysis reveal a negative treatment effect—i.e., if the estimated treatment group difference in the 28-item PSPRS score for AMX0035 compared to placebo is ≤0—the study will be terminated for futility and the Phase 3 portion will not be initiated.

If, however, the applicable Phase 2b analysis shows a positive treatment effect for AMX0035, participants randomized in that portion will continue on either their double-blind study drug assignment or on open-label AMX0035 (as applicable), and the Phase 3 study portion will be initiated. Additional details will be provided in an amendment to the protocol. Refer to Section “Interim, Primary, and Final Analyses” for additional information.

### Phase 3 Study Portion

As noted above, the Phase 3 study portion will utilize the same design as in Phase 2b—i.e., a double-blind phase, followed by an optional OLE phase for eligible participants who consent to continue on in the study. Participants in the Phase 2b study portion will not be eligible to take part in the Phase 3 portion.

### Phase 3 Double-Blind Phase

The Phase 3 double-blind phase will be identical in design to the Phase 2b double-blind phase—i.e., a Screening Period of up to 6 weeks; a double-blind Treatment Period of up to 52 weeks; and a remote Safety Follow-up visit occurring 4 weeks (+7 days) after the EOT visit.

Participants in the Phase 3 double-blind phase will be randomized in a 3:2 ratio to receive either AMX0035 or matching placebo, and will receive their first dose in the clinic. Participants will be instructed to take 1 dose daily and then increase to 1 dose twice daily (BID; taken morning and evening) starting at the Phase 3 Week 2 visit. The Week 2 visit can be conducted either at the clinic or via telephone or a telehealth virtual platform, based on the Investigator's assessment and the participant's agreement.

Participants will return to the clinic for assessments at Week 4, Week 12, Week 24, Week 36, and Week 52 of the Phase 3 portion.

### Phase 3 OLE Phase

At the Phase 3 Week 52 visit, participants in the Phase 3 double-blind phase may consent to roll over into the OLE phase as long as they continue not to meet any of the study discontinuation criteria (as per Section “Discontinuation from Study Treatment”) in the judgment of the Investigator. The Week 52 visit will serve as the baseline visit for the OLE phase.

As in the Phase 2b study portion, the OLE phase of the Phase 3 portion will be up to 52 weeks in duration. Eligible consenting participants will receive their first dose of open-label AMX0035 in the clinic under the supervision of site personnel. Participants will be instructed to take 1 dose of open-label AMX0035 daily and then increase to 1 dose BID (taken morning and evening) starting at the Phase 3 Week 54 visit. The Week 54 visit can be conducted either at the clinic or via telephone or a telehealth platform, based on the Investigator's assessment and the participant's agreement.

Participants in the Phase 3 OLE phase will remain in the study for an additional 52 weeks in total, and will return to the clinic for assessments at Week 56, Week 76, and Week 104. Participants will then meet with study staff via telephone or a telehealth platform for the Safety Follow-up visit at Week 108.

### Phase 3 Interim Analysis

If the Phase 3 portion of the study is ultimately conducted, a preplanned IA will be performed when approximately 40% of the total information from that portion becomes available—i.e., when 40% of the participants have completed 52 weeks of study treatment, have withdrawn from study treatment, or have died. Refer to Section “Interim, Primary, and Final Analyses”) for details.

### Phase 3 Primary Analysis

The Phase 3 primary analysis will be performed when all participants in that portion have completed 52 weeks of study treatment, have withdrawn from study treatment, or have died. Refer to Section “Interim, Primary, and Final Analyses” for details.

### Phase 3 Final Analysis

As detailed in Section “Interim, Primary, and Final Analyses”, survival status will be assessed for every study participant until their death or until the end of the study is announced, whichever should occur first.

The final Phase 3 analysis, which is to be focused on overall survival, is planned to be performed 5 years after the last participant has completed study treatment.

### Participant Involvement in Design of the Study

People living with PSP, caregivers of people with PSP, and PSP disease advocacy representatives from the United States and Europe provided feedback to a draft version of the protocol. This feedback was incorporated into the original design of this study.

### Start of Study and End of Study

The start of the study is defined as the date of site activation, when the Investigator is given the authority to recruit participants. The start of recruitment is defined as the first visit of the first participant, which equates to the date of signature on the first informed consent form for participation in the study.

The end of trial is defined as the date on which the last participant completes survival follow-up.

### Study Population

Note: The inclusion and exclusion criteria listed below apply to both the Phase 2b and Phase 3 study portions.

### Inclusion Criteria

To be eligible for this study, participants must meet all of the following criteria throughout the Screening Period:
1. Provides a signed informed consent form (ICF) and has the mental capability to understand it. If participant is unable to sign the ICF, the ICF must be signed by a representative in accordance with local regulatory requirements.
2. Is willing and able to comply with the scheduled visits, treatment schedule, laboratory tests, and other requirements of the study, including MRI scans.
3. Participant's study partner is willing and able to comply with scheduled visits, treatment schedule, laboratory tests, and other requirements of the study. The participant's study partner is defined as a caregiver, family member, social worker, or friend who has frequent contact with the participant (˜10 hours per week), will accompany the participant to study visits to provide information as to the participant's functional abilities, and will speak the local language fluently to ensure comprehension of informed consent and informant-based assessments of participant.
4. Is male or female, 40 to 80 years of age, inclusive.
5. Participant must reside outside a skilled nursing facility or dementia care facility at the time of Screening and admission to such a facility must not be planned. Residence in an assisted living facility is allowed.
6. Meets the following criteria for possible or probable PSP (Steele-Richardson-Olszewski Syndrome) according to MDS 2017 criteria (Höglinger 2017):
   a. Gradually progressive disorder, with age at disease onset ≥40 years.
   b. Either or both of the following two criteria are met:
      i. Vertical supranuclear gaze palsy OR slow velocity of vertical saccades AND postural instability with repeated unprovoked falls within 3 years OR tendency to fall on the pull-test within 3 years.
      ii. Slow velocity of vertical saccades AND postural instability with more than two steps backward on the pull-test within 3 years.
7. Presence of PSP symptoms for <5 years (as determined by the best judgment of the Investigator). For the purpose of this inclusion criterion, a PSP symptom is defined as any neurological, cognitive, or behavioral symptom consistent with known symptoms of PSP, occurring newly and subsequently progressing during the clinical course in the absence of another identifiable cause.
8. Score of <40 on the total (28-item) PSPRS Score.
9. Is able to walk independently or with minimal assistance, defined as the ability to walk 5 steps with minimal assistance (stabilization of one arm).
10. Score of ≥24 on the Mini Mental State Examination (MMSE).
11. Dosing of anti-Parkinsonian drugs (coenzyme Q10, levodopa/carbidopa, levodopa/benserazide, fava bean extract, a dopamine agonist, catechol-O-methyltransferase inhibitor, amantadine, or other Parkinson's disease medications) should be stable for 60 days before enrollment and anticipated to remain stable for the double-blind phase of the Phase 2b or Phase 3 study portion, as applicable to the participant, at the discretion of the Investigator.
12. All female participants must have a negative serum pregnancy test at Screening.
13. Female participants of childbearing potential (women of childbearing potential [WOCBP]) who engage in heterosexual intercourse must have a negative urine pregnancy test on Day 1 prior to the start of the study.
14. WOCBP must agree to abstain from heterosexual intercourse or use a highly effective birth control method for the duration of the study and for 6 months after the last dose of study drug. Female participants who are two years postmenopausal or surgically sterile are not considered to be of childbearing potential.
15. Female participants must not be planning to become pregnant for the duration of the study and for 6 months after the last dose of study drug.
16. Female participants must not be planning to breastfeed or be breastfeeding for the duration of the study and for 6 months after the last dose of study drug.
17. Male participants must agree to abstain from heterosexual intercourse or use a highly effective birth control method for the duration of the study and for 6 months after the last dose of study drug.
18. Male participants must not be planning to father a child or provide sperm for donation for the duration of the study and for 6 months after the last dose of study drug.

Exclusion Criteria Participants will not be eligible for this study if they meet any of the following criteria:
1. Has previous exposure to AMX0035 or has a known hypersensitivity to AMX0035, either of its components, any of its excipients, or bile salts. Note that under this exclusion criterion, participants in the Phase 2b study portion are not eligible to take part in the Phase 3 portion.
2. Requires use of a feeding tube.
3. Evidence of any neurological disorder that could explain signs of PSP, including any of the following:
   a. Signs of idiopathic Parkinson's disease (e.g., severe asymmetric parkinsonian signs, clinically significant tremor at rest, or prominent and sustained response to levodopa therapy or other medications that are used to treat Parkinson's disease).
   b. Signs of multiple system atrophy (MSA) (e.g., prominent early cerebellar limb ataxia or unexplained symptomatic autonomic dysfunction).
   c. Signs of Lewy body disease (e.g., hallucinations or delusions unrelated to dopaminergic therapy or other illness).
   d. Probable Alzheimer's disease (AD) according to National Institute of Aging-Alzheimer's Association (NIA-AA) core clinical criteria for mild cognitive impairment due to AD or AD dementia.
   e. History of repeated strokes with stepwise progression of Parkinsonian features.
   f. History of major stroke.
   g. History of severe or repeated head injury.
   h. History of encephalitis.
   i. History of neuroleptic use within the past 6 months; clozapine or quetiapine may be permitted if at a stable dose for 60 days prior to Screening.
   j. History of oculogyric crises.
   k. History of street-drug-related Parkinsonism.
4. Any contraindication to MRI or abnormal findings evidenced by MRI, including any of the following:
   a. Severe leukoencephalopathy.
   b. Relevant structural abnormalities (normal pressure or obstructive hydrocephalus) including basal ganglia, diencephalic, mesencephalic, pontine, or medullary infarctions, hemorrhages, hypoxic-ischemic lesions, tumors, or malformations.
   c. Arteriopathy with subcortical infarcts and leukoencephalopathy (CADASIL).
   d. Severe cerebral amyloid angiopathy.
5. History of autosomal dominant PSP due to a Microtubule Associated Protein Tau (MAPT) mutation.
6. History of an autosomal dominant mutation associated with Frontotemporal Lobar Degeneration (FTLD) (e.g., an autosomal dominant mutation in C90RF72 or GRN).
7. Abnormal clinical laboratory results, including any of the following findings (at Screening only):
   a. Abnormal liver function, defined as aspartate transaminase (AST) and/or alanine transaminase (ALT)>3×the upper limit of normal (ULN).
   b. Renal insufficiency, as defined by estimated glomerular filtration rate (eGFR)<60 mL/min/1.73 m².
   c. Ongoing anemia with hemoglobin concentration<10.0 g/dL.
8. Current biliary disease which may lead to biliary obstruction or impaired biliary flow, including active cholecystitis, primary biliary cirrhosis, sclerosing cholangitis, gallbladder cancer, gallbladder polyps, gangrene of the gallbladder, or abscess of the gallbladder.
9. History of Class III/IV heart failure per New York Heart Association (NYHA) criteria.
10. Clinically significant infection, inflammation, or medical condition other than PSP that would pose a risk to the participant or impair their ability to participate in the study, in the judgment of the Investigator, including:
   a. Acute gastrointestinal symptoms (e.g., nausea, vomiting, diarrhea) at time of Screening or on Day 1 prior to study drug dosing.
   b. Presence of pathologies that can alter the enterohepatic circulation of bile acids (e.g., ileal resection and stoma, regional ileitis).
   c. Severe salt restriction, where added salt intake due to treatment with study drug would put the participant at risk in the judgment of the Investigator.
11. Evidence of any clinically significant neurological disorder other than PSP, including significant cerebrovascular abnormalities, vascular dementia, motor neuron disease or ALS, Huntington's disease, normal pressure hydrocephalus, brain tumor, seizure disorder, multiple sclerosis, or known structural brain abnormalities. Evidence of disease may be provided by MRI.
12. Prior or current diagnosis of schizophrenia, schizoaffective disorder, or bipolar disorder, according to Diagnostic and Statistical Manual of Mental Disorders, 5th Edition (DSM-V) or International Classification of Diseases (ICD)-10 criteria.
13. Presence of unstable psychiatric disease, cognitive impairment (e.g., major cognitive dysfunction), dementia, major depression, or substance abuse that would impair ability of the participant to provide informed consent and follow instructions, in the judgment of the Investigator.
14. Significant suicidal ideation within 1 year prior to Screening as evidenced by answering “yes” to questions 4 or 5 on the suicidal ideation portion of the Columbia-Suicide Severity Rating Scale (C—SSRS) at Screening or history of suicidal attempts within the last 2 years.
15. Participation in any other clinical investigation using an experimental drug within 5 half-lives or within 6 weeks (small molecules) or 6 months (monoclonal antibodies, antisense oligonucleotides, or other biologics), whichever is longer, prior to Day 1.
16. Exposure to gene or cell therapy prior to Screening or during study.
17. Exposure to any prohibited therapy within 30 days prior to Screening.
18. Any factor which, in the opinion of the Investigator, precludes the participant's full compliance with or completion of this study.

### Lifestyle Considerations

### Contraception

All WOCBP and male participants must agree to abstain from heterosexual intercourse or use a highly effective method of contraception for the duration of the study and for 6 months after their last dose of study drug.

The following guidelines for contraception are consistent with the Clinical Trials Facilitation and Coordination Group (CTFG) Recommendations related to contraception and pregnancy testing in clinical trials, version 1.1 (CTFG 2020).

### Definitions

A female participant is considered to be a WOCBP (i.e., fertile) following menarche and until becoming postmenopausal, unless permanently sterile. Permanent sterilization methods include hysterectomy, bilateral salpingectomy, and bilateral oophorectomy.

A postmenopausal state is defined as no menses for 12 months without an alternative medical cause. A follicle-stimulating hormone (FSH) test at Screening is required to confirm a menopausal or postmenopausal state in women not using hormonal contraception or hormonal replacement therapy. However, in the absence of 12 months of amenorrhea, a single FSH measurement is insufficient.

A male participant is considered fertile after puberty unless permanently sterile by bilateral orchidectomy.

### Birth Control Methods

Birth control methods which may be considered highly effective (failure rate of less than 1% per year when used consistently and correctly) include the following:
Combined (estrogen- and progesterone-containing) hormonal contraception associated with inhibition of ovulation.
Progesterone-only hormonal contraception associated with inhibition of ovulation
Intrauterine device (IUD).
Bilateral tubal occlusion.
Vasectomized partner, provided that partner is the sole sexual partner of the WOCBP trial participant, and the vasectomized partner has received medical assessment of the surgical success.
Sexual abstinence, defined as refraining from heterosexual intercourse.

Birth control methods which may not be considered highly effective include:
Progesterone-only hormonal contraception, where inhibition of ovulation is not the primary mode of action.
Male or female condom, with or without spermicide.
Cap, diaphragm, or sponge, with or without spermicide.

### Prior and Concomitant Therapy

Ongoing concomitant medication (prescription or over-the-counter, including vitamins and herbal supplements) use and any prior medication administered within the last 6 months prior to Screening will be recorded. All concomitant medications, including any change in dose, must be recorded with the name of the medication, dosage information including dose, route and frequency, date(s) of administration including start and end dates, and reason for use.

All allowable concomitant medications must be at a stable dose for at least 30 days prior to the participant's Screening visit, and it is anticipated that no change in dose will be required during the study treatment period. All medications should remain at stable doses for the duration of the study unless a change in regimen is medically necessary.

The Medical Monitor should be contacted if there are any questions regarding concomitant and prior therapy(ies) or prohibited medications. The Medical Monitor must be notified if administration of any prohibited medication is reported during the study.

### Prohibited Therapy

Except for the study drug, any investigational therapy being used or evaluated for the treatment of PSP is prohibited beginning 30 days (or 5 half-lives, whichever is longer) prior to the study first dose and throughout the treatment period.

Use of any gene or cellular therapy prior to this study excludes participants from enrollment. These are also prohibited during the study.

Throughout the course of the study, participants should not be treated with medications listed in Table 4 below; these medications are prohibited therapies due to a potential drug-drug interaction (DDI) when combined with AMX0035. If an Investigator learns that a participant has begun therapy with any of these medications, this should be reported to the Medical Monitor immediately and the Investigator and Sponsor should make the determination whether to stop the study drug or the prohibited medication immediately, taking into account the health, safety, and preference of the participant.

TABLE 4
Drugs Prohibited During A35-009 (ORION) Study Participation (due to Potential Drug-Drug Interaction when Combined with AMX0035)
Prohibited Drug Groups* (refer to note immediately above table)

| | | | |
|---|---|---|---|
| Antacids containing aluminum | Anticoagulants, including the following: (refer to | Benzodiazepines/ | Bile acid and derivatives, |
| hydroxide or smectite | note immediately below regarding anticoagulants in | gamma-aminobutyric acid | whether as a supplement, |
| (aluminum oxide) within two | relation to lumbar puncture) | (GABA) agonist examples*, | over-the-counter drug, or |
| (2) hours before or after | Apixaban | including the following. Such | prescription drug: |
| administration of study drug. | Dabigatran | medications are prohibited | UDCA |
| These antacids may inhibit | Enoxaparin sodium | within 30 days prior to | TURSO/Taurourso- |
| absorption of TURSO. They | Heparin | Screening and throughout the | deoxycholic acid |
| include: | Rivaroxaban | duration of the study: | (TUDCA) |
| Alamag | Vitamin K antagonists | Chlordiazepoxide | |
| Alumina and Magnesia | Note: The Investigator should discuss with the | Diazepam | |
| Antacid, Antacid M and | Medical Monitor any questions or concerns about | Flurazepam | |
| Antacid Suspension | potentially interrupting, adjusting, or stopping a | Meprobamate | |
| Gen-Alox | participant's anticoagulant therapy in preparation for | Midazolam | |
| Kudrox | the lumbar puncture procedure. | Temazepam | |
| Magnesium-aluminum | In certain cases, the Investigator and Medical | Triazolam | |
| hydroxide (MAH) | Monitor may jointly determine that the lumbar | | |
| Maalox heartburn relief | puncture will not be performed for that participant; | | |
| formula (HRF) and Maalox | any such instance is to be recorded as a protocol | | |
| therapeutic concentrate | deviation. | | |
| (TC) | | | |
| Magnalox | | | |
| Madroxal | | | |
| Mylanta and Mylanta | | | |
| Ultimate | | | |
| Ri-Mox | | | |
| Rulox | | | |
| Bile acid sequestrants, | For the cytochrome P450 enzymes and | HDAC inhibitors, including: | Neuroleptics *, including the |
| including: | transporters listed below, the particular drugs | Butyrate | following. Such medications |
| Cholestyramine and | listed, unless otherwise specified, are prohibited | Chidamide | are prohibited within 30 |
| Cholestyramine Light | due to the combination of potential inhibition by | Lithium | days prior to Screening and |
| Colestid and Colestid | AMX0035 and a narrow therapeutic index of these | Panobinostat | throughout the duration of |
| Flavored | drugs. Prohibition of these drugs would lower the | Romidepsin | the study: |
| Prevalite | risk of a potential drug-drug interaction that may | Suramin | Chlorpromazine |
| Questran and Questran | result from a potential pharmacokinetic and/or | Valproate | Fluphenazine |
| Light | pharmacodynamic interaction with AMX0035: | Vorinostat (Zolinza) | Haloperidol |
| Welchol | Substrates of CYP2C8, CYP2C9, CYP2C19, | | Loxapine |
| | CYP2B6, CYP1A2, and CYP3A4/5: | | Perphenazine |
| | Carbamazepine | | Thioridazine |
| | Masitinib | | Thiothixene |
| | Methadone | | Trifluoperazine |
| | Olanzapine | | |
| | Phenytoin | | |
| | Pioglitazone | | |
| | Quinidine | | |
| | Rasagiline | | |
| | Warfarin | | |
| | Substrates of P-glycoprotein | | |
| | (P-gp) and breast cancer resistance protein | | |
| | (BCRP): | | |
| | Apixaban | | |
| | Dabigatran | | |
| | Digoxin | | |
| | Tamoxifen | | |
| | Substrates of organic anion transporter (OAT)1 | | |
| | and OAT3: | | |
| | Methotrexate | | |
| | Penicillins | | |
| | Rifampicin | | |
| | Inhibitors of OATP1B3 including the following are | | |
| | prohibited: | | |
| | Cyclosporin | | |
| | Gemfibrozil | | |

```
Other Prohibited Drugs:
  Acetyl-L-Carnitine
  Antisense therapy
  Fludrocortisone
  Inosine
  Probenecid for potential kidney interaction
Note:
Drug groups marked with an asterisk (*) exclude compounds used on an as-needed basis, as described in Section “Magnetic Resonance Imaging” and Section “Lumbar Puncture”.
```

### Additional Guidance on Concomitant Medication

Drugs commonly known to cause excessive sedation, orthostatic hypotension, or increased risk for falls should be avoided.

In general, dose change or administration of additional medications with psychotropic effects (including opiates) on an as-needed (i.e., PRN) basis is prohibited. Low doses of anxiolytic/hypnotic agents or antipsychotic or opiate-containing medications are permitted in the interest of participant safety or emergent symptom control; however, ongoing use of PRN medications should be discussed with the Medical Monitor.

If a participant requires PRN use of a medication with psychotropic effects, participant efficacy scales must not be administered at least 48 hours following the administration of the medication. Depending on the time of dose administration, the next study visit should be rescheduled to assure an interval of at least 48 hours between PRN medication administration and clinical or psychometric assessments. Each instance of PRN medication administration must be documented with the reason for use, dates of administration, and dosages in the eCRF. A participant's daily scheduled medication(s) should not be delayed and administration times should not be altered due to cognitive assessments.

### Other Restrictions and Precautions

### 1. Thickeners

The use of thickening agents is not permitted during drug administration.

### 2. Sodium Content of AMX0035

The study drug has a high sodium content. In participants sensitive to salt intake, consider the amount of daily sodium intake and monitor appropriately.

### 3. MRI Contraindications

The imaging specialist at the study site's MRI facility is responsible for determining if a participant is contraindicated from having this procedure. The following is a list of some common conditions that may preclude the participant from having MRI scans. However, this should not be used as a substitute for local clinical standard of care. The ultimate decision to perform the MRI rests with the site radiologist, the Investigator, and the standards set by the local Independent Review Board (IRB)/Independent Ethics Committee (IEC):
A history of claustrophobia
An MRI-incompatible pacemaker, epicardial pacemaker wires, incompatible cardiac valve prostheses, and MRI-incompatible vascular clips less than 2 months old or MRI-incompatible aneurysm clips of any age
An MRI-incompatible cochlear implant
An MRI-incompatible spinal nerve stimulator
An MRI-incompatible infusion pump
Metallic fragments in the eyes/orbits or in the vicinity of the brain or major neurovascular structures of the body
An employment history that involves exposure to welding, unless absence of metallic fragments is documented by X-ray examination as per institutional practice
Shrapnel in/at any place in the body

### Screen Failures and Rescreening

Screen failures are defined as participants who consent to participate in the clinical study but are not subsequently assigned to study drug (i.e., AMX0035 or matching placebo). A minimal set of screen failure information will be entered into the electronic data capture system, including demographics (demographic information will consist of participant date of birth [or age], sex, race, and ethnicity [where applicable]); screen failure details; eligibility criteria; and any SAE(s).

Rescreening of a participant may be performed up to 2 times. If re-screened, the participant must be re-consented. The assessments to be conducted during re-screening will be determined after consultation with the Medical Monitor.

Retesting of laboratory parameters and/or other assessments will be permitted after consultation with the Medical Monitor. Additionally, re-testing may be performed to confirm a value. The most current result prior to treatment is the value by which study inclusion will be assessed.

### Discontinuation From Study Treatment

A participant may withdraw their participation or may be discontinued from study treatment at any time, at the discretion of the Investigator or termination of the study by the Sponsor, regulatory agency, or IRB/IEC. The primary reason for discontinuation must be recorded in the participant's electronic case report form (eCRF). Every reasonable effort will be made to complete final assessments and discharge procedures and to provide medical follow-up as indicated. The Investigator will notify the Sponsor of the withdrawal of any participant from the study.

A participant must permanently discontinue from study treatment for any of the following reasons:
The participant becomes pregnant. Study treatment must be discontinued immediately. Report the pregnancy according to the instructions in Section “Pregnancy”.
If the participant refuses or cannot continue with a study procedure, the Investigator should review the case with the Sponsor and Medical Monitor before discontinuing the participant from study treatment.
The participant experiences an AE that necessitates permanent discontinuation from a study drug.
At the discretion of the Investigator for medical reasons.
The participant is non-compliant with study drug or study procedures, and the Investigator or Sponsor determines that the non-compliance is a risk to the participant if continued in the study treatment period.
The participant's study partner can no longer satisfy the study requirements and an alternate study partner cannot be identified and consented.

For the purpose of study withdrawal criteria, baseline will be considered the most recent predose assessment.

Participants who discontinue from study treatment will have the EOT visit as close to the stop of study drug (i.e., AMX0035 or matching placebo) as possible (within 7 days of the participant's last dose). The Safety Follow-up visit (telephone call) will occur at least 28 days (+7 days) after the last dose of study drug to monitor the outcome/resolution of the AEs that were present upon discontinuation. Participants who withdraw from study treatment prior to Week 52 visit are not eligible to participate in the OLE phase of the study.

The participant will also remain active in the study for the important periodic long-term survival status reporting to the study site staff (see Section “Long-term Survival Assessment”).

Withdrawal from the Study

A participant may withdraw their consent to participate in the study at any time. Additionally, the participant may be withdrawn from the study at any time at the discretion of the Investigator or Sponsor. The primary reason for discontinuation of study must be recorded in the participant's eCRF. Participants who withdraw from the study during the double-blind phase of either the Phase 2b or Phase 3 portion of the study (as applicable) will not be eligible to participate in the OLE phase of that study portion.

No further contact with the participant will occur for a participant after they withdraw consent or are withdrawn from the study.

The early withdrawal date is defined as the date of the decision by either the participant, Investigator, or Sponsor to withdraw the participant from the study.

### Lost to Follow-Up

Lost to follow-up is defined by the inability to reach the participant after a minimum of three (3) documented phone calls, faxes, or emails as well as lack of response by participant to one registered mail letter. All attempts should be documented in the participant's medical records.

If it is determined that the participant has died, the site will use permissible local methods to obtain date and cause of death.

The site staff and representative will consult publicly available sources, such as public health registries and databases, in order to obtain updated contact information.

If after all attempts, the participant remains lost to follow-up, then the last known alive date as determined by the Investigator should be reported and documented in the participant's medical records.

### Study Treatment

### Delivery of Product to Site Pharmacy and Product Labeling

AMX0035 will be supplied by the sponsor to the site pharmacy as a carton box containing 60 single use sachets.

Each 10 g sachet contains 3 g PB and 1 g TURSO as active ingredients.

### Excipients Include:

Sodium phosphate dibasic, anhydrous
Dextrates, Hydrates
Sorbitol
Syloid 63FP (colloidal silica)
Sucralose
Sodium stearyl fumarate
Weber Mixed Berry Flavoring
Kleptose Linecaps (maltodextrin)

If at any point during the study, circumstances should arise that prevent participants from completing in-clinic visits (e.g., due to medical condition, or in the event that precautions are needed to mitigate risk during a pandemic, natural disaster, or social or political unrest), the Sponsor may implement direct-to-participant or direct-from-participant study drug shipment via a Sponsor-approved courier service or via site courier service per institutional policies.

### Product Storage and Stability

All study drug supplies are stored in a locked, safe area with access limited to authorized site personnel. Specific instructions on product storage and stability will be provided in the study-specific Clinical Pharmacy Manual.

### Dosage, Preparation, and Administration of AMX0035

Study drug will be administered orally according to the regimen provided in Table 5. The initial dose level will be 1 sachet of study drug daily in the morning. In the absence of the emergence of intolerable diarrhea or nausea, and if an overall benefit-risk evaluation is favorable, participants will be instructed to increase the regimen to one sachet of study drug twice a day in the morning and evening at the Week 2 visit. Otherwise, the once daily regimen will be maintained with regular monitoring every 4 weeks until resolution enables dose escalation after discussion between the Investigator and Medical Monitor.

TABLE 5
A35-009 (ORION) Study Drug ^{a} Dosing Regimen - Phase
2b and Phase 3 (if conducted) Study Portions

| Dosing Level | Dosing Frequency ^{b} |
|---|---|
| Initial dose: ^{c} | One (1) sachet daily: |
| Double-blind study phases (Day 1 to | Mornings |
| the Week 2 visit) | |
| OLE study phases (Week 52 to the | |
| Week 54 visit) | |
| Maintenance dose: ^{b} | Two (2) sachets daily: |
| Double-blind study phases (Week 2 | One (1) sachet in the morning |
| visit to EOT) | AND |
| OLE study phases (Week 54 visit to | One (1) sachet in the evening |
| EOT) | |

^{a} “Study drug” refers to either AMX0035 or matching placebo in the double-blind study phases and to open-label AMX0035 in the OLE phases.
^{b} Participants will be encouraged to administer study drug at similar times in the morning and evening throughout the study.
^{c} In the case of diarrhea or sustained nausea occurring, participants will be monitored every 4 weeks until resolution. Dose escalation will commence after resolution and after discussion between the Investigator and Medical Monitor (refer to Section “Modification (including Interruption or Discontinuation) of Study Drug for a Participant”).
Abbreviations: EOT = End of Treatment; OLE = open-label extension.

On Day 1, authorized site personnel will prepare the participant's study drug (i.e., AMX0035 or matching placebo, as applicable in the double-blind study phases) per the following instructions:

Open one (1) sachet of AMX0035 (or matching placebo); pour the powder in a cup or other container; add approximately 8 oz/250 mL (1 cup) of room-temperature water; and stir vigorously. Note that it is normal for a small amount of powder to remain undissolved.

The site personnel will provide oral instructions to the participants regarding preparation of study drug and will supervise the first oral administration of study drug.

Participants should consume within 1 hour after the powder has been placed in the cup or other container and the room-temperature water has been added. Participants may use a small amount of water to rinse (1 oz/30 mL) the bitter taste of the study drug.

The use of thickening agents is not permitted during study drug administration.

It is required that the study drug be taken on an empty stomach prior to a meal or snack. Normal eating and drinking are allowed until 1 hour before and 1 hour after study drug dose administration (morning and evening doses).

Eligible participants who consent to continue into the OLE phases of the study will receive their first dose of open-label AMX0035 under the supervision of site personnel. Dosing in the OLE study phases will follow the schedule outlined in Table 5.

Specific instructions on the product dispensing schedule and recordkeeping will be provided in the study-specific Clinical Pharmacy Manual.

### Disposal of Study Drug at the Completion of the Study

At the completion of the study, there will be a final reconciliation of study drug shipped, drug consumed, and drug remaining. Any discrepancies noted will be investigated, resolved, and documented prior to destruction or return of unused study drug.

Methods for disposal of study drug are provided in the study-specific Clinical Pharmacy Manual.

### Randomization

At Screening, each participant will be assigned a unique participant number. Participant numbers will be assigned consecutively using interactive response technology (IRT).

Randomization for treatment with either AMX0035 or matching placebo will be independently developed by an unblinded statistician. All participants will be centrally randomized using IRT.

On Day 1, participants who meet all of the inclusion and none of the exclusion criteria upon Screening will be randomized 3:2 into 2 treatment groups: AMX0035 or matching placebo. Before the study is initiated, each user will receive log-in information and directions on how to access the IRT.

Randomization will be stratified by region (North America or Europe).

### Blinding

In the event of a medical emergency, the Investigator may unblind an individual participant's study drug allocation. Instructions for unblinding an individual participant's study drug allocation are contained in the study-specific IRT manual. Unblinding should only occur if management of the medical emergency would be different based on the participant having received study drug. In non-emergency situations where unblinding is being considered, the site should discuss the situation with the Medical Monitor.

If a site unblinds the treatment assignment for a participant, the study drug must be discontinued for that participant. The participant should remain in the study and complete all study assessments except those directly related to dosing.

In cases of accidental unblinding, the Investigator will contact an unblinded Sponsor representative and ensure every attempt is made to preserve the blind.

Throughout their participation in the OLE study phases (of the Phase 2b and Phase 3 study portions), Investigators, study staff, and the participants themselves will remain blinded to the study treatment—i.e., AMX0035 or matching placebo—to which participants were assigned in the double-blind study phases.

In the Phase 2b study portion, quarterly safety monitoring will be conducted internally by the Medical Monitor, using blinded data outputs. The predefined unblinded team will be unblinded at the time of the Phase 2b IA database lock.

In the Phase 3 study portion, an external IDMC will perform a periodic review of safety data; this IDMC will also review unblinded efficacy data at the Phase 3 IA and provide recommendations concerning the continuation, modification, or stoppage of the Phase 3 portion for safety or efficacy reasons.

### Modification (Including Interruption or Discontinuation) of Study Drug for a Participant

Dose-limiting TEAEs may be managed by dose interruption, dose modifications, and discontinuation of study drug for participants. The decision to modify dosing, interrupt dosing, or discontinue the study drug administration should be performed based on clinical judgement of the Investigator and the overall benefit-risk for the participant.

In situations where Investigators may use the NCI CTCAE toxicity rating as an optional guide for their decision to modify or interrupt the dose or discontinue the study drug for participants, the following recommendations may be considered:

In participants with dose-limiting TEAEs of NCI CTCAE grade <3, intolerable to the participant and possibly related to study drug in the opinion of the Investigator (including, but not limited to persistent diarrhea, vomiting or nausea, signs of dehydration, increase in serum creatinine or liver enzymes of lower severity), the event(s) may be managed by a temporary interruption or dose reduction to a lower dose of 1 sachet of study drug per day per the discretion of the Investigator. The reduced dose, or interruption may be maintained for as long as necessary until the event improves. The Investigator may then choose to resume the higher dosage or maintain the participant at a reduced dosage. Any dose interruptions should be discussed with the Medical Monitor.

For dose-limiting TEAEs of NCI CTCAE grade ≥3, possibly related to the study drug in the opinion of the Investigator, the study drug should be interrupted until the resolution of AEs or the event improves to NCI CTCAE grade <3. The Investigator may then choose to resume at the higher dosage or maintain the participant at a reduced dosage of 1 sachet of study drug per day. Any participant experiencing recurrent TEAE(s) of NCI CTCAE grade ≥3, possibly related to the study drug in the opinion of the Investigator, upon resumption within a reasonable time period should be discontinued from further administration of study drug in the study.

For all dose interruptions or dose modifications due to a possibly related TEAE, the study drug should be discontinued if the Investigator believes that it is in the best interest of the participant to discontinue the study drug for safety reasons, irrespective of whether NCI CTCAE toxicity grading is used.

Treatment emergent adverse events (TEAE) that are intolerable to the participant and possibly related to study drug in the opinion of the Investigator (including, but not limited to persistent diarrhea, vomiting or nausea, signs of dehydration, increase in serum creatinine or liver enzymes of lower severity than described below) may be managed by a dose reduction to one sachet of AMX0035 per day The Investigator may decide at any time to interrupt treatment; however, any changes to the dosing regimen must be recorded in the eCRF. If TEAEs persist, further reductions must be discussed with the medical monitor.

If a participant demonstrates treatment-emergent signs of neurotoxicity, including, but not limited to, vomiting, nausea, headache, dizziness, somnolence, dysgeusia, hypoacusis, disorientation, confusion, memory loss, or neuropathy, that are possibly related to study drug in the opinion of the Investigator, the Investigator should consider a dose reduction or interruption.

The reduced dose, or interruption may be maintained for as long as necessary until the event improves. The Investigator may then choose to resume the higher dosage or maintain the participant at a reduced dosage. Any dose interruptions must be discussed with the trial Medical Monitor.

Recurrence of the dose-limiting TEAE upon re-introduction of the study drug may result in the permanent discontinuation of study drug.

The following TEAEs will trigger temporary dose interruption for the participant:
Treatment-emergent increase in serum creatinine or liver enzymes:
   Serum creatine>50% increase from Baseline
   ALT or AST>8×upper limit of normal range (ULN)
   ALT or AST>5×ULN for more than 2 weeks
   ALT or AST>3×ULN AND
      Serum total bilirubin >2×ULN OR international normalized ratio >1.5
   ALT or AST>3×ULN with the appearance of fatigue, nausea, vomiting, right upper quadrant pain or tenderness, fever, rash, and/or eosinophilia (>5%)
Grade 3 TEAE per NCI Common Terminology Criteria for Adverse Events (CTCAE) version 5.0 (NCI CTCAE v5.0) unless otherwise specified

Any dosage adjustment, including the reason for and dates of adjustment, will be documented in the source documentation and eCRF. Any dose modification may be discussed with the trial Medical Monitor.

In case of an overdose, defined as absorption of a daily dose equivalent to >15 g PB per day (>5 sachets/day), treatment should be immediately discontinued and supportive measures implemented as medically indicated.

### Treatment Compliance

Treatment compliance will be assessed at every scheduled visit.

For clinic visits, participants will be instructed to return used and unused study drug sachets in their original container(s) at each clinic visit and at the EOT visit. The number of sachets dispensed and returned (used and unused) will be recorded by the Investigator at each visit to obtain information on participants' compliance.

The participant will be considered compliant with treatment if ≥80% and <125% of the total scheduled doses since the last clinic visit have been verified. If a participant is non-compliant with administration of study drug, the Investigator should re-educate and re-train the participant in administration of study drug and evaluate if a dose reduction is required. Each incidence of non-compliance will be reported as a minor protocol deviation.

### Study Assessments and Procedures

### Recruitment and Screening

The first on-site contact with the individual will occur either a few days before screening for the participants to take the ICF and review it at home or at the Screening visit where informed consent will be obtained.

The Investigator will obtain written informed consent prior to each participant's participation in the study. Participants will be screened for eligibility in a sequential fashion and may be re-screened up to 2 times if they fail the screen at any point.

All screening evaluations must be completed within 6 weeks prior to Day 1. The Investigator will maintain a screening log to record details of all participants screened and to confirm eligibility or record reasons for screening failure, as applicable.

If the individual meets all the eligibility criteria, the participant will be contacted and, if he/she remains willing to participate in the study, he/she will be instructed to return to the clinic on Day 1 to confirm eligibility. The participant will also be reminded of the restrictions that are agreed to in the consent and listed in inclusion/exclusion criteria.

The participant will be instructed to contact the site personnel if there are any questions regarding these restrictions or if any of these restrictions have been breached.

### Identification of Study Partner

To be eligible for screening, each prospective participant must have an identified, reliable, study partner (e.g., caregiver, family member, social worker, or friend), who has frequent contact with the participant (˜ or >10 hours per week) and who will accompany the participant to study visits to provide information as to the participant's functional abilities.

The designated study partner must be sufficiently familiar with the participant in order to provide accurate data and be willing and able to perform all the responsibilities of a study partner for the duration of the study, as determined by the Investigator. The site must obtain the name and contact information of the study partner. The Investigator must obtain written informed consent from the study partner prior to the participant's participation in the study.

Study partners are required to be present for all study visits.

If the study partner can no longer satisfy the study requirements, then an alternate study partner must be identified and consented to the study. If an alternate study partner cannot be identified, then the participant would be forced to discontinue from study treatment as per Section “Discontinuation from Study Treatment”.

### Safety Assessments

As noted previously, quarterly safety monitoring in the Phase 2b study portion will be conducted internally by the Medical Monitor, using blinded data outputs. The predefined unblinded team will be unblinded at the time of the Phase 2b IA database lock.

In the Phase 3 study portion, an external IDMC will perform a periodic review of safety data; this IDMC will also review unblinded efficacy data at the Phase 3 IA and provide recommendations concerning the continuation, modification, or stoppage of the Phase 3 portion for safety or efficacy reasons.

Safety monitoring will involve vital signs (temperature, heart rate, and blood pressure), 12-lead electrocardiogram (ECG), safety laboratory testing, physical examination, and documentation of AEs.

For both the Phase 2b and Phase 3 study portions, planned timepoints for all safety assessments are presented in the Schedules of Activities for the double-blind (Table 1) and OLE (Table 2) study phases.

### Medical History

For all participants, a complete medical history will be obtained at Screening. Medical history includes collection of demographics (demographic information will consist of participant date of birth [or age], sex, race, and ethnicity [where applicable]) as well as the participant's history of PSP and any medications taken for symptoms related to PSP. In addition, a history of alcohol and tobacco use will be obtained from each participant.

The medical history will be updated on Day 1. The updated medical history on Day 1 will serve as the baseline for clinical assessment purposes (i.e., not for statistical analysis purposes, for which “baseline” will be defined in the SAP).

### PSP Clinical Features

A detailed assessment of possible or probable PSP (Steele-Richardson-Olszewski Syndrome) signs and symptoms present at Screening will be collected and include the following (Höglinger 2017):
Ocular motor dysfunction
Postural instability
Akinesia
Cognitive dysfunction

Based on participant's medical history, Investigators will also be asked to provide the participant's clinical phenotype as outlined in the MDS 2017 criteria (Höglinger 2017). PSP clinical phenotypes may include the following:
PSP with predominant Richardson syndrome
PSP with predominant ocular motor dysfunction
PSP with predominant postural instability
PSP with predominant parkinsonism
PSP with progressive gait freezing
PSP with predominant frontal presentation
PSP with predominant speech/language disorder
PSP with predominant corticobasal syndrome

### Physical Examination

A complete physical examination will be performed at Screening per institutional policy and must include at least the following body systems:
Head, eyes, ears, nose, throat
Neck, chest (including heart and lungs)
Abdomen (including gastrointestinal system)

For both the Phase 2b and Phase 3 study portions, physical examinations at subsequent visits—as outlined in the Schedules of Activities for the double-blind and OLE study phases (Table 1 and Table 2, respectively)—may be directed to relevant clinical findings.

The physical examination performed prior to the participant's first dose of study drug during the baseline visit will serve as the baseline physical examination for clinical assessment purposes (i.e., not for statistical analysis purposes, for which “baseline” will be defined in the SAP).

Any significant physical examination findings after dosing will be recorded as AEs.

Body weight will be measured as indicated in the Schedule of Activities. The participant will wear lightweight clothing and no shoes during weighing. Height will be measured at Screening only; the participant will not wear shoes.

### Medical Resource Utilization

Medical resource utilization data that is associated with medical encounters will be collected as part of the physical examination. Protocol-mandated procedures, tests, and encounters are not included.

The medical resource utilization data collected may include:
Date and duration of medical care encounters.
The purpose of the medical care encounters, including if they were related to PSP disease and/or treatment.
Character of medical encounters, i.e., physician office visit, specialist office visit, emergency room visit, surgeries, tests and procedures, physical therapy, speech and swallow therapy, occupational therapy, etc.
Number and character of resources used during the medical care encounters, including:
   Fall-related remedies
   Swallowing-related remedies
   Tools for vision support
   Respiratory aids
   Support for neurological events, including for dementia
   Tools for voice assistance and communication
If the medical care encounter resulted in hospitalization.

### Vital Signs

Vital signs will include temperature, heart rate, and blood pressure. The position of the participant (i.e., supine, semi-supine, orthostatic) during blood pressure and heart rate assessments is at the discretion of the Investigator, and these assessments must be preceded by 5 minutes of rest for the participant. Blood pressure and heart rate will be assessed using the arm opposite to that used for blood draws.

For visits in which both vital signs and blood sample(s) are collected, vital signs should be obtained prior to any blood collection.

The vital signs measurements taken prior to the first dose during the baseline visit will serve as the baseline vital signs measurements for clinical assessment purposes (i.e., not for statistical analysis purposes, for which “baseline” will be defined in the SAP).

For visits in which both vital signs and blood sample(s) are collected, vital signs should be obtained prior to any blood collection.

### Neurological Examination

For both the Phase 2b and Phase 3 study portions, a brief standard neurological examination will be performed at the times indicated in the Schedules of Activities for the double-blind and OLE study phases (Table 1 and Table 2, respectively).

The neurological exam performed prior to the first dose during the baseline visit will serve as the baseline neurological exam for clinical assessment purposes (i.e., not for statistical analysis purposes, for which “baseline” will be defined in the SAP).

Symptoms identified during the Screening Period will not be recorded as AEs; however, new symptoms or current symptoms that change in severity or frequency after the first day of study drug will be recorded as AEs. The neurological examination will assess:
Mental status—assessment of orientation, speech, and memory
Cranial nerves—assessment of cranial nerves II-XII. This will include an assessment of supranuclear gaze palsy and slowing of vertical saccades.
   Vertical supranuclear palsy will be established by neurological examination demonstrating a >50% limitation of the range of voluntary gaze in the vertical plane, which is overcome by reflexive vestibular stimulation.
   Slowing of vertical saccades may be assessed by either of the two following methods:
      Slowing of vertical saccades may be established by neurological examination of saccades toward a target held >20 degrees from the position of primary gaze in the vertical plane.
   Slowing of vertical saccades will be defined as present when ocular movement is slow enough for the examiner to see its progress, rather than just its initial and final positions. A delay in initiation of saccades is not considered slowing.
      Slowing of vertical saccades may be established using quantitative measurements of saccades, such as infrared oculography of adequate spatial and temporal resolution to resolve multiple saccades.
Motor system—brief assessment of tone and strength, tremors
Sensory system—brief assessment of light touch and temperature sensation
Reflexes—assessment of deep tendon reflexes and plantar responses (Babinski sign)
Coordination—assessment of upper and lower extremities, including assessment for tremor
Gait—assessment of tandem gait (if clinically indicated and safe)
Station—assessment of posture and stability as defined by the following:
   If clinically indicated and safe, postural instability may be assessed by determining the impairment of postural reflexes on neurological examination (i.e., retropulsion with or without unaided recovery after a backward pull) in the absence of any other medical cause to explain this impairment (e.g., primary sensory deficit, vestibular dysfunction, pontine infarction, cerebellar syndrome, prominent upper or lower motor neuron signs).

### Electrocardiogram

Two 12-lead electrocardiograms (ECGs) will be obtained as indicated in the Schedule of Activities. ECGs will be recorded after the participant has been supine for at least 5 minutes. The participant should be instructed to remain completely stationary during the recording, without talking, laughing, deep breathing or swallowing during the time of recording (10 seconds). For visits in which both ECGs and blood sample(s) are collected predose, ECGs should be obtained prior to any blood collection, with the blood draw taken immediately (within 5-10 minutes) following the ECG trace.

The ECG measurements obtained prior to the participant's first study drug dose during the baseline visit will serve as the baseline ECG measurements for clinical assessment purposes (i.e., not for statistical analysis purposes, for which “baseline” will be defined in the SAP).

All ECGs will be read by a qualified local physician at the study site. The qualified local physician will interpret and document his/her global interpretation on the ECG tracing, based on the following conventions, as appropriate:
Normal ECG
Abnormal ECG—not clinically significant
Abnormal ECG—clinically significant

This physician will sign and date the ECG tracings. Each ECG should be reviewed by the physician before study drug administration to ensure the tracing is interpretable and no acute, medically serious condition is present.

The Investigator's (or physician designee's) initial interpretation of the ECG will be the basis of any decisions related to the study conduct and treatment of the study participants (e.g., eligibility at baseline, AE assessment, etc.).

### Clinical Laboratory Tests

The tests detailed in Table 6 will be performed by a central laboratory. Additional tests may be performed for safety reasons at any time during the study as determined necessary by the Investigator. Post-study blood samples will be taken ±1 hour from the nominal blood sampling time except when the time point coincides with the PR blood sampling time. In this situation, the time window for the PR sample applies.

TABLE 6
Protocol-required Safety Laboratory Assessments

| Laboratory Assessments | Parameters | |
|---|---|---|
| Hematology | Complete blood count (CBC), including | Mean cell hemoglobin concentration |
| | differential: | (MCHC) |
| | hemoglobin, hematocrit, red blood cell | Mean corpuscular hemoglobin (MCH) |
| | (RBC) count, white blood cell (WBC) | Mean corpuscular volume (MCV) |
| | count, platelet count, mean platelet | |
| | volume (MPV), and absolute and relative | |
| | numbers of neutrophils, lymphocytes, | |
| | monocytes, eosinophils, and basophils | |
| Clinical Chemistry | Alanine aminotransferase (ALT)/serum | Lactate dehydrogenase (LDH) |
| | glutamic-pyruvic transaminase (SGPT) | Magnesium |
| | Albumin | Potassium |
| | Alkaline phosphatase (ALP) | Sodium |
| | Aspartate aminotransferase (AST)/serum | Thyroid Stimulating Hormone (TSH) |
| | glutamic-oxaloacetic transaminase | Thyroxine (FT4) |
| | (SGOT) | Total and direct bilirubin |
| | Bicarbonate/carbon dioxide (CO₂) | Total protein |
| | Blood urea nitrogen (BUN) | Urea |
| | Calcium | Uric acid |
| | Chloride | Vitamin B12 (cobalamin) |
| | Creatinine, including creatinine kinase | |
| | Glucose | |
| Urinalysis | Glucose | Protein |
| (dipstick - reflex to | Ketones | Red blood cells |
| laboratory urine testing as | pH | Specific gravity |
| required) | | |
| Coagulation ^{a} | Partial thromboplastin time (PTT) or | Prothrombin time/ International |
| | activated partial thromboplastin time | Normalized Ratio (PT/INR) |
| | (aPTT) | |
| Drugs of abuse (collected | 2-ethyl-1,5-dimethyl-3,3- | Cannabinoids |
| via urine) ^{b} | diphenylpyrrolidine (EDDP) | Cocaine metabolites |
| | Amphetamine | Opiates |
| | Barbiturates | Phencyclidine (PCP) |
| | Benzodiazepines | |
| Serum Pregnancy Test for | β-hCG | |

| | |
|---|---|
| all female participants | |
| Pregnancy | All female participants: β-hCG Screening and Week 52 (if this is an EOT visit or if |
| | participant does not continue into OLE), and Week 104 only |
| | WOCBP, only: urine pregnancy test (treatment period visits) |
| | If indicated: FSH |

^{a} The coagulation assessment is only required prior to each study lumbar puncture and must be processed by the central laboratory. The determination of timing for the coagulation assessment relative to each lumbar puncture is to be guided by institutional procedure and Investigator discretion.
^{b} Screening visit only. A positive result should be discussed with the Medical Monitor in order to rule out potentially confounding medications.
c. A serum pregnancy test will be performed at the Screening visit and at the End of Treatment visit for all female participants.

The Investigator or delegated personnel must review all laboratory reports, document this review, and record any clinically significant findings occurring during the study in the participant record and AE section of the eCRF. The laboratory reports must be filed with the source documents.

All laboratory testing after the dose of study drug with abnormal values considered clinically significant should be repeated until the values return to normal or baseline, are no longer considered clinically significant, or are deemed chronically stable by the Investigator or Medical Monitor. If such values do not return to normal/baseline within a period of time judged reasonable by the Investigator, the etiology should be identified, and the Sponsor notified.

### Efficacy Assessments

For both the Phase 2b and Phase 3 study portions, efficacy assessments are to be performed in accordance with the Schedules of Activities for the double-blind and OLE study phases (Table 1 and Table 2, respectively).

### Magnetic Resonance Imaging

For both the Phase 2b and Phase 3 study portions, MRIs will be collected during the double-blind and OLE study phases as indicated in Table 1 and Table 2, respectively, while following procedures outlined in the study-specific Imaging Manual. The time window for the MRI procedure at each protocol-required timepoint is ±7 days.

The Screening MRI should be completed after all other relevant screening procedures have been completed and reviewed by the Investigator.

The Screening MRI assessment will be performed to assess eligibility and will be read locally as per facility's standard of care. The local reading will be used to evaluate the presence of any intracranial masses that might preclude the participant from undergoing a lumbar puncture and exclude focal or diffuse processes that could indicate a clinically significant neurologic disorder other than PSP.

Scans at all protocol-required timepoints will be uploaded to the central imaging vendor. The central imaging vendor will perform imaging analyses.

Scans at all protocol-required timepoints that are of poor quality (e.g., due to participant motion during the scan, inadequate coverage of the brain, improper positioning of the head, use of incorrect scan or geometry parameters, or noisy image) as determined by the MRI technologist at the imaging center, the reviewing radiologist, or central imaging vendor will be repeated at the earliest possible time but within 15 days of the protocol required timepoint.

Sedation is not recommended but is allowed after discussion by the participant and the Investigator prior to the imaging procedure. If sedation is required, it should occur after all scales and cognitive testing have been performed or, if this is not possible, at least 48 hours before all scales and cognitive testing are scheduled.

### Lumbar Puncture

For both the Phase 2b and Phase 3 study portions, lumbar punctures will be collected during the double-blind and OLE study phases as indicated in Table 1 and Table 2, respectively. Each lumbar puncture must be performed >3 months from the previous lumbar puncture. A lumbar puncture at Week 104/EOT is optional.

Lumbar punctures will be performed by qualified, experienced practitioners and per institutional procedures. At the discretion of the Investigator, contraindications to lumbar puncture should be reviewed according to site policies. Instructions around specimen processing will be provided within the Laboratory Manual.

In general, lumbar punctures should be performed in the morning (between 08:00 and 12:00 hours) whenever possible in order to minimize potential diurnal variation of cerebrospinal fluid (CSF) parameters.

If sampling is not successful or is not standard of care for the institution, other methods, including CT/fluoro-guided or ultrasound-guided lumbar puncture, may be used at the discretion of the local clinical site staff. CSF samples will be analyzed by an applicable designated laboratory for AMX0035, tau, and other exploratory biomarkers.

Institutional policy and Investigator discretion should be followed to provide the appropriate post-lumbar puncture observation period and to provide information to the participant on possible side effects and limitations on strenuous physical activity and driving.

### Diagnostic Tools and Rating Scales

Rater training and certification will be required for the administration of selected scales and assessments. These requirements will be outlined in the study-specific rating manuals and any supplemental materials.

It is the responsibility of the Investigator to ensure that the raters at his/her site are appropriately trained and certified in the use of selected rating scales and assessments. Every effort must be made by the sites to ensure that each participant is rated by the same rater for each scale or assessment throughout their participation in the study.

Administration of selected scales may be audio recorded (as permitted by local regulations) to allow for central review of the data and to ensure consistency and reliability.

Outcome measures will be performed by an appropriately qualified individual.

Each of the participant-reported outcome measures may be administered as clinician-reported outcome measures where allowable by the assessment's author/developer. As such, the assessments will be completed by a qualified member of the site staff, who will be reporting information collected from the participant and/or the study partner as appropriate in order to promote consistent quality, standardization of test, and minimize burden on the study participant and/or study partner.

For both the Phase 2b and Phase 3 study portions, assessments will be performed as indicated in Table 1 and Table 2 for the double-blind and OLE study phases, respectively.

Study tools and scales are to follow the predefined order of administration indicated in Table 3. Note that the tools and scales designated with an “X” within the table may be collected in any order.

TABLE 3
A35-009 (ORION) Diagnostic Tools and Scales - Order of Administration

| | | | | | | | | | Week 104/ |
|---|---|---|---|---|---|---|---|---|---|
| | | | | | | | | | End of |
| | Screening | Baseline ^{b} | Week 4 | Week 12 | Week 24 | Week 36 | Week 52/ | Week 76 | Treatment |
| Diagnostic Tools | | Day 1 | | | | | | | |
| and Scales | | | | | | | | | |
| PSPRS (28-item) ^{a, c} | 1 | 1 | 1 | 1 | 1 | 1 | 1 | 1 | 1 |
| PSPRS (10-item) ^{a, c} | | 2 | 2 | 2 | 2 | 2 | 2 | 2 | 2 |
| MDS-UPDRS Part II ^{a} | 2 | 3 | 3 | 3 | 3 | 3 | 3 | 3 | 3 |
| SE-ADL ^{a} | | 4 | | 4 | 4 | 4 | 4 | 4 | 4 |
| MoCA | | 5 | | | 5 | | 5 | 5 | 5 |
| CGI-S ^{a} | | X | | X | X | X | X | X | X |
| CGI-C ^{a} | | | | X | X | X | X | X | X |
| MMSE ^{a, d} | X | | | | | | | | |
| EQ5D-5L ^{a} | | X | | | X | | X | X | X |
| C-SSRS ^{a, e} | X | X | | X | X | X | X | X | X |
| ZBI ^{f} | | X | | | X | | X | X | X |

Note:
Numbering presented in this table provides a predefined order of administration that should apply to each visit. Tools and scales designated with an “X” may be administered in any order.
^{a} Scales should be prioritized ahead of other study procedures at each unique visit.
^{b} Assessments are to be performed prior to the participant's first dose of study drug.
^{c} The 28-item PSPRS should be obtained prior to the 10-item PSPRS. At Screening, only the 28-item PSPRS will be performed.
^{d} MMSE will be administered only during Screening to assess eligibility.
^{e} The C-SSRS “Lifetime/Recent” version will be administered at Screening. The C-SSRS “Since Last Visit” version will be administered at all subsequent visits.
^{f} ZBI will be conducted, but there is no mandatory timing for this assessment in relation to the timing of the other study procedures conducted at each unique visit.
Abbreviations: CGI-C = Clinical Global Impressions Scale - Change; CGI-S = Clinical Global Impressions Scale - Severity; C-SSRS = Columbia Suicide Severity Rating Scale; EQ-5D-5L = EuroQoL 5 Dimension 5-Level; MDS-UPDRS = Movement Disorder Society-Unified Parkinson's Disease Rating Scale; MoCA = Montreal Cognitive Assessment; MMSE = Mini Mental State Examination; PSPRS = Progressive Supranuclear Palsy Rating Scale; SE-ADL = Schwab and England Activities of Daily Living.

### PSP Rating Scale

The PSPRS is a quantitative measure of disability in patients with PSP (Golbe 2007). The full PSPRS includes 28 items in six domains. The available total score ranges from 0 (normal) to 100 (maximally disabled). Six items are rated on a 3-point scale (0 to 2) and 22 items are rated on a 5-point scale (0 to 4). The History/Daily Activities area includes seven items with a total maximum of 24 points, the Mentation area four items with 16 points, the Bulbar area two items with 8 points, the Ocular Motor area four items with 16 points, the Limb Motor area six items with 16 points, and the Gait area five items with 20 points. In addition, in line with United States Food and Drug Administration request, a modified version of the PSPRS (10-item PSPRS) that includes only Items 3, 4, 5, 12, 13, 24, 25, 26, 27, 28 from the 28-item PSPRS scale with a modified scoring algorithm will be assessed in this study (provided in the study-specific rating manuals and any supplemental materials).

When both the 28-item and 10-item PSPRS assessments are scheduled, the 28-item PSPRS should be obtained prior to the 10-item PSPRS. At Screening, only the 28-item PSPRS will be performed.

### Movement Disorder Society-Unified Parkinson's Disease Rating Scale Part II

The MDS-UPDRS is made up of 5 parts; however, only Part II will be administered in this study.

The MDS-UPDRS Part II includes 13 items assessing motor aspects of experiences of daily living (Goetz 2007).

These items include speech, saliva and drooling, chewing and swallowing, handwriting, doing hobbies and other activities, eating tasks, tremor, dressing, hygiene, turning in bed, getting out of bed, walking and balance, and freezing.

### Schwab and England Activities of Daily Living Scale

The SE-ADL scale is a means of assessing a person's ability to perform daily activities in terms of speed and independence through a percentage figure (Schwab 1969).

The SE-ADL is composed of two sections.

The first section is a self-report questionnaire in which participants grade their own daily life activities, such as dressing, using the toilet, resting, eating, and social activities (subjective assessment). The rating will be determined by a qualified staff member according to the participant's self-reported functional ability of specific criteria, with 100% indicating total independence and falling to 0% indicating a state of complete dependence.

The second section is an assessment of motor functions, such as postural balance, speaking, rigidity, and tremors, and will be conducted by a clinician (objective assessment).

### EuroQuality of Life

The EuroQol EQ-5D-5L contains a health state descriptive part comprising five items, scored from 1 (no problems or symptoms) to 3 (serious problems or symptoms), a question about change in health state in the preceding 12 months, and a visual analog scale (VAS) to evaluate current health state (from 0, worst imaginable, to 100, best imaginable). The descriptive profile can be converted into a value (EQ-Index) which ranges from 0 (death) to 1 (perfect health), with negative values indicating health states considered worse than death.

### Zarit Burden Interview

The Zarit Scale of Caregiver Burden or the Zarit Burden Interview is used to assess caregiver burden. The short version contains 22 items. Each item of the interview is a statement which the consented study partner of the participant is asked to endorse using a 5 point scale. Response options range from 0 (Never) to 4 (Nearly Always).

### Clinical Global Impression of Severity and Change

The CGI-S is a clinician's rating of disease severity. The CGI-S rates severity of illness on a 7-point scale, using a range of responses from 1 (normal) through to 7 (the most severely ill). This rating is based upon observed and reported symptoms, behavior, and function in the past 7 days.

The CGI-C rates improvement by 7 categories: very much improved, much improved, minimally improved, no change, minimally worse, much worse, very much worse.

The CGI-C will be assessed relative to the CGI-S at the Week 1 (Day 1) visit as the reference.

### Mini-Mental State Exam

The MMSE is a brief, 30-point questionnaire that provides a quantitative measure of cognitive status in adults and is widely used to screen for cognitive impairment and to estimate the severity of cognitive impairment at a given point in time. The MMSE total score ranges from 0 to 30, with lower score indicating greater impairment (Folstein 1975).

The MMSE will be used only for inclusion criterion purposes. The participant must have a score of ≥24 at screening to be eligible for study participation.

### Montreal Cognitive Assessment

The MoCA was designed as a rapid screening instrument for mild cognitive dysfunction (Nasreddine 2005). It assesses different cognitive domains: attention and concentration, executive functions, memory, language, visuoconstructional skills, conceptual thinking, calculations, and orientation.

### Columbia-Suicide Severity Rating Scale

The C—SSRS is a systematically administered instrument developed to track suicidal AEs across a treatment study. The instrument is designed to assess suicidal behavior and ideation, track and assess all suicidal events, as well as the lethality of attempts. Additional features assessed include frequency, duration, controllability, reason for ideation, and deterrents. The C—SSRS is considered a low-burden instrument as it takes less than 5 minutes to administer.

The C—SSRS “Lifetime/Recent” version will be administered at Screening. For both the Phase 2b and Phase 3 study portions, the C—SSRS “Since Last Visit” version will be administered at all subsequent clinic visits as indicated within the Schedules of Activities for the double-blind and OLE study phases (Table 1 and Table 2, respectively).

Any participant noted to have suicidal ideation with plan within the prior month, either via answering “yes” to Questions 4 or 5 to the suicidal ideation portion of the C—SSRS or via clinical interview, will be evaluated immediately by the Investigator.

The Medical Monitor and Sponsor will also be informed. Appropriate steps will be taken to protect the participant, including but not limited to possible discontinuation (decided by either the Investigator or Medical Monitor) from the study and referral for appropriate psychiatric care. Any such participant at Screening or on Day 1 will also be excluded from the study.

### Pharmacokinetic and Biomarker Assessments

For both the Phase 2b and Phase 3 study portions, blood and CSF samples will be collected as indicated in the Schedules of Activities for the double-blind and OLE study phases (Table 1 and Table 2, respectively).

Coded samples will be frozen and stored in a secure storage space with adequate measures to protect confidentiality prior to PK and biomarker analysis.

The samples may be retained while research on AMX0035 or PSP continues, but for no longer than 20 years from the end of the study per local requirements.

For the first 110 participants randomized in the study, blood samples will be collected to assess the exposure to PB, TURSO, and their respective metabolites following multiple doses of AMX0035. PK analysis of these samples may occur after the study is completed. Refer to Section “Pharmacokinetic Sampling” immediately below for details.

Blood and CSF samples collected during the study may be used to evaluate known and/or novel disease-related or drug-related biomarkers.

Instructions for collection, processing, storage, and shipping of samples will be provided in the study-specific Laboratory Manual.

### Pharmacokinetic Sampling (Applicable to First 110 Randomized Participants Only)

For PK sampling purposes, venous blood samples will be collected from the first 110 participants randomized in the study. These samples will be collected either by venipuncture or an indwelling cannula inserted in a forearm vein. The actual date and time (24-hour clock time) of each sample will be recorded. The time of the last meal prior to administration and the time/date of the drug administration(s) in the previous 24 hours will also be noted.

On Day 1 and during the Week 24 and Week 52 visits, blood samples for PK will be collected from each of the first 110 participants randomized in the study. The collection timepoints at these visits are approximately 1, 4, and 6 hours postdose (±20 minutes). A PK sample will not be taken from any of these 110 participants at an Early Termination visit if that participant discontinued study drug more than 48 hours prior to the visit.

### Biomarker Samples

For all participants in the study, blood samples (approximately 11 mL) will be collected no more than 30 minutes prior to the study drug administration on Day 1. Blood samples collected at Week 24 and Week 52 may be collected at any time during that visit.

CSF samples (18 to 20 mL) will be collected by lumbar puncture on Day 1 and at Week 52. CSF sample collection at Week 104/EOT is optional.

### Long-term Survival Assessment

Following completion of or discontinuation from study treatment, ongoing reporting of survival status will be recorded for each study participant until either the participant's time of death or the end of the study is announced.

Contact for determination of long-term survival can be accomplished by direct (e.g., participant or study partner) or indirect contact (e.g., national death rolls or social security data among others). Direct contact will be performed by the site staff no more than every 12 weeks following the completion of or discontinuation from study treatment for long-term survival assessments.

### Safety and Adverse Events

### Definition and Classification of Adverse Events

An AE is any untoward medical occurrence in a participant that occurs once a medicinal product has been administered, including occurrences which are not necessarily caused by or related to that product. A treatment-emergent AE (TEAE) is defined as an AE where the onset date is on or after the treatment start date. An adverse drug reaction (ADR) is any AE where a causal relationship with the study drug is at least a reasonable possibility (possibly, probably, or definitely related).

The severity of AEs should be assessed according to the NCI CTCAE version 5.0 grading scale (NCI CTCAE), or as follows when no appropriate code is relevant:
Grade 1—Mild AE that is easily tolerated by the participant, causes minimal discomfort, and does not interfere with everyday activities
Grade 2—Moderate AE that is sufficiently discomforting to interfere with normal everyday activities; intervention may be needed
Grade 3—Severe AE that prevents normal everyday activities; treatment or other intervention usually needed
Grade 4—Very severe or life-threatening
Grade 5—Death

### Assessment of Causality

The relationship of the AE to the study drug/investigational product should be specified by the Investigator, using the following definitions:
Not Related: Concomitant illness, accident, or event with no reasonable association with treatment.
Unlikely Related: The reaction has little or no temporal sequence from administration of the study drug/investigational product and/or a more likely alternative etiology exists.
Possibly Related: The reaction follows a reasonably temporal sequence from administration of the study drug/investigational product and follows a known response pattern to the suspected study drug/investigational product. The reaction could have been produced by the study drug/investigational product or could have been produced by the participant's clinical state or by other modes of therapy administered to the participant (Suspected ADR).
Probably Related: The reaction follows a reasonably temporal sequence from administration of study drug/investigational product, is confirmed by discontinuation of the study drug/investigational product or by re-challenge, and cannot be reasonably explained by the known characteristics of the participant's clinical state (Suspected ADR).
Definitely Related: The reaction follows a reasonable temporal sequence from administration of study drug/investigational product that follows a known or expected response pattern to the study drug/investigational product. The reaction is confirmed by improvement on stopping or reducing the dosage of the study drug/investigational product and reappearance of the reaction on repeated exposure (Suspected ADR).

### Recording Adverse Events

Adverse events will be recorded from Day 1 until 28 days (+7 days) after the last dose of study drug (at the Follow-up Phone Call is acceptable). Serious AEs (see Section “Serious Adverse Events” below) will be recorded from the time of signed informed consent until 28 days (+7 days) after the last dose of study drug (at Safety Follow-up is acceptable).

Events that may be attributable to the progression and/or defining features of PSP are to be reviewed by the Investigator and site staff to determine whether they meet the criteria for reporting as an AE. This may, for example, include situations in which the participant is progressing at a significantly faster pace than could be reasonably expected, or is presenting with atypical disease symptoms. Any such PSP-related event meeting AE criteria is to be reported appropriately within participant source documentation and EDC.

During each study visit, the participant will be questioned directly regarding the occurrence of any adverse medical event and documented in the participant's record and the eCRFs. All AEs, whether ascribed to study procedures or not, will be documented immediately. This will include the date of onset, a description of the AE, severity, duration, actions taken, outcome, and the Investigator's current opinion on the relationship between the study drug and the event. A diagnosis and final opinion on the relationship between the study drug and the event will be provided at the end of the study by the Investigator.

### Fall Event Reporting

Falls are a core component and defining feature of PSP. As falls are reported, site staff will determine if the falls meet one or more of the following criteria that require the fall to be reported as an AE:
Lead to other injury
Represent a change in frequency or character from pre-randomization falls
Necessitate a healthcare visit
Result in hospitalization

The following additional details should be collected for all fall events meeting AE criteria, as applicable:
Type of medical intervention required (i.e., sutures, sling, cast, wheelchair)
Type of healthcare visit necessitated (i.e., physical therapy, emergency room visit)
Type of medical diagnostic procedure required (i.e., blood draw, x-ray, stress test)
If the fall resulted in hospitalization

Falls meeting AE criteria will be reported appropriately within participant source documentation and EDC.

### Serious Adverse Events

An SAE is defined as any untoward medical occurrence that at any dose:
Results in death
Is life-threatening (defined as an event in which the participant was at risk of death at the time of the event; it does not refer to an event which hypothetically might have caused death if it were more severe)
Requires hospitalization or prolongation of existing hospitalization
   NOTE: The following hospitalizations are not considered SAEs in Amylyx clinical studies:
      A visit to the emergency room or other hospital department <24 hours that does not result in admission (unless considered an important medical or life-threatening event)
      Elective surgery, planned prior to signing consent
      Admissions as per protocol for a planned medical/surgical procedure
      Routine health assessment requiring admission for baseline/trending of health status (e.g., routine colonoscopy)
      Medical/surgical admission other than to remedy ill health and planned prior to entry into the study. Appropriate documentation is required in these cases.
      Admission encountered for another life circumstance that carries no bearing on health status and requires no medical/surgical intervention (e.g., lack of housing, economic inadequacy, caregiver respite, family circumstances, administrative reason).
Results in persistent or significant disability or incapacity
Consists of a congenital anomaly or birth defect.
An important medical event (defined as a medical event(s) that may not be immediately life threatening or result in death or hospitalization but, based upon appropriate medical and scientific judgment, may jeopardize the participant or may require intervention [e.g., medical, surgical] to prevent one of the other serious outcomes listed in the definition above). Examples of such events include, but are not limited to, intensive treatment in an emergency room or at home for allergic bronchospasm, blood dyscrasias, or convulsions that do not result in hospitalization.)

SAEs must be reported to the Sponsor within 24 hours of awareness by emailing the SAE form to: safety@amylyx.com (refer to Section “Regulatory Reporting Requirements for Serious Adverse Events”).

### Deaths

Death is an outcome of an event. The event that resulted in death should be recorded on the appropriate CRF. All proximal causes of death must be reported as SAEs within 24 hours of the site becoming aware of the event. The Investigator should make every effort to obtain and send death certificates and autopsy reports to Amylyx Pharmaceuticals. The term death should be reported as an SAE only if the cause of death is not known and cannot be determined.

### Suspected Unexpected Serious Adverse Reactions

Suspected unexpected serious adverse reactions (SUSARs) are AEs that are believed to be related to the study drug and are both unexpected (i.e., the nature or severity is not expected) and serious. SUSARs are participant to expedited reporting (refer to Section “Regulatory Reporting Requirements for Serious Adverse Events”). The Sponsor will report electronically and without delay to the EudraVigilance database all relevant information about any SUSAR.

### Method of Detecting Adverse Events and Serious Adverse Events

Care will be taken to not introduce bias when detecting AEs and/or SAEs. Open-ended and non-leading verbal questioning of the participant is the preferred method to inquire about AE occurrences.

### Follow-Up of Adverse Events and Serious Adverse Events

If only limited information is initially available, follow-up for AEs/SAEs is required until resolution or stabilization. If an ongoing SAE changes in its intensity or relationship to study drug, or if new information becomes available, the SAE report must be updated and submitted within 24 hours to the Sponsor using the same procedure used for transmitting the initial SAE report.

Any participant who discontinues from the study due to an AE will be followed until the outcome is determined and in the case of an SAE, written reports provided by the Investigator.

### Regulatory Reporting Requirements for Serious Adverse Events

Prompt notification from the Investigator to the Sponsor of SAEs within 24 hours of the site becoming aware of the event is essential so that legal obligations and ethical responsibilities towards the safety of participants and the safety of a product under clinical investigation are met.

An Investigator who receives an Investigator safety report describing SAEs or other specific safety information (e.g., summary or listing of SAEs) from the Sponsor will file it along with the Investigator's Brochure and will notify the IRB/IEC, if appropriate according to local requirements.

The Sponsor will report AEs to regulatory authorities and ethics committees according to local applicable laws. A SUSAR is a subset of SAEs and will be reported to the appropriate regulatory authorities and Investigators following local and global guidelines and requirements.

### Pregnancy

If a pregnancy is reported, the Investigator should inform the Sponsor within 24 hours of learning of the pregnancy.

Abnormal pregnancy outcomes (e.g., spontaneous abortion, fetal death, stillbirth, congenital anomalies, ectopic pregnancy) are considered SAEs.

Male Participants with Partners Who Become Pregnant

The Investigator will attempt to collect pregnancy information on any male participant's female partner who becomes pregnant while the male participant is in this study. This applies only to male participants who receive study drug (i.e., AMX0035 or matching placebo).

After obtaining the necessary signed informed consent from the pregnant female partner directly, the Investigator will record pregnancy information on the appropriate form and submit it to Amylyx within 24 hours of learning of the partner's pregnancy. The female partner will also be followed to determine the outcome of the pregnancy. Information on the status of the mother and child will be forwarded to the Sponsor. Generally, the follow-up will be no longer than 6 to 8 weeks following the estimated delivery date. Any termination of the pregnancy will be reported regardless of fetal status (presence or absence of anomalies) or indication for the procedure.

### Female Participants Who Become Pregnant

The Investigator will collect pregnancy information on any female participant who becomes pregnant while participating in this study. Information will be recorded on the appropriate form and submitted to Amylyx within 24 hours of learning of a participant's pregnancy.

The participant will be followed to determine the outcome of the pregnancy. The Investigator will collect follow-up information on the participant and the neonate and the information will be forwarded to the Sponsor. Generally, follow-up will not be required for longer than 6 to 8 weeks beyond the estimated delivery date. Any termination of pregnancy will be reported, regardless of fetal status (presence or absence of anomalies) or indication for the procedure.

While pregnancy itself is not considered to be an AE or SAE, any pregnancy complication or elective termination of a pregnancy will be reported as an AE or SAE. A spontaneous abortion is always considered to be an SAE and will be reported as such.

Any post-study pregnancy related SAE considered reasonably related to the study drug (i.e., AMX0035 or matching placebo) by the Investigator will be reported to Amylyx. While the Investigator is not obligated to actively seek this information in former participants, he or she may learn of an SAE through spontaneous reporting.

### Statistical Considerations

Data from participants in the Phase 2b study portion and data from participants in the Phase 3 portion will be kept distinct for the purposes of statistical analysis, inference of the treatment effect, and evaluating decision rules at the time of the prespecified Phase 2b IA.

The SAP for the Phase 2b portion will be finalized before the Phase 2b IA database lock. This SAP will describe, in detail, the analysis sets to be included in the applicable analyses, as well as procedures to account for missing data.

A separate SAP for the Phase 3 study portion, if that portion is ultimately conducted, will be finalized before the Phase 3 IA database lock.

### Analysis Populations

The Intention-to-Treat (ITT) Population will comprise all randomized participants, regardless of whether or not study drug (i.e., AMX0035 or matching placebo) was actually administered. This population will be based on the treatment group to which the participant was randomized.

The Safety Population will consist of all participants who received at least one dose of study drug and procedures for accounting for missing, unused, and spurious data. will be based on the actual study drug received, if this differs from that to which the participant was randomized.

### Sample Size Determination

### Phase 2b Sample Size Determination

For the Phase 2b study portion, 110 participants will be randomly assigned in a 3:2 ratio to receive AMX0035 or placebo (360 participants in the either AMX0035 or matching placebo (N=66 participants in the AMX0035 treatment group and 24044 participants in the placebo group). These participants will go on to complete the 52-week double-blind phase of the Phase 2b portion, regardless of the timing of the Phase 2b IA and primary analysis.

Anticipating a dropout rate of approximately 25% (based on previous clinical studies conducted in participants individuals with PSP), approximately 26650 participants in the AMX0035 treatment group and 177 approximately 33 participants in the placebo group are expected to complete the Phase 2b portion of this study through Week 52. The Phase 2b portion is exploratory and is not powered to demonstrate efficacy of AMX0035 compared to placebo (as demonstrated by PSPRS score). The sample size of 110 participants in the Phase 2b portion provides a reasonable chance of detecting futility under various scenarios.

### Phase 3 Sample Size Determination

The final decision whether to initiate the Phase 3 study portion will be based on the totality of available data from the Phase 2b IA or Phase 2b primary analysis, including available data for all efficacy-, safety-, and biomarker-related endpoints. In an example scenario for Phase 3, participants (N=approximately 600 to 800) would be randomly assigned in a 3:2 ratio to receive AMX0035 or matching placebo in the double-blind phase.

Using a two-sided, two-sample t-test with one-sided alpha level set at 0.025, this sample size with a sample size of N=600 (360 in the AMX0035 group and 240 in the placebo group)-assuming a dropout rate of approximately 25%-270 participants in the AMX0035 group and 180 participants in the placebo group, all having completed the study procedures through Week 52, will provide 90% power to detect a difference of 3.2 points for AMX0035 relative to placebo in the change in 28-item PSPRS total score from baseline to Week 52 for AMX0035 relative to placebo, (assuming a common standard deviation of 10 in the change in PSPRS total score from baseline to Week 52.).

The sample size for the Phase 3 study portion is subject to change based on the totality of available data from the Phase 2b IA or Phase 2b primary analysis. The exact sample size determination for the Phase 3 portion (as applicable) will be included in a future amendment to the protocol after either the Phase 2b IA or the Phase 2b primary analysis is complete.

### Interim, Primary, and Final Analyses

### Phase 2b Analyses

The enrollment of the Phase 2b study portion will be considered complete when all of the participants in that portion have been randomized. A preplanned IA will be performed when all of these randomized participants have completed 24 weeks of study treatment, have withdrawn from study treatment, or have died.

The primary analysis for the Phase 2b study portion will be performed when all of the participants randomized in that portion have completed 52 weeks of study treatment, have withdrawn from study treatment, or have died.

The final analysis for the Phase 2b study portion will be performed after the study is considered complete. This analysis will focus on overall survival.

### Criteria and Process for Determining Whether to Initiate Phase 3 Study Portion

If a negative treatment effect is observed in either the IA or primary analysis performed for the Phase 2b study portion (i.e., estimated treatment group difference in PSPRS score for AMX0035 compared to placebo is ≤0), the study will stop immediately for futility and no Phase 3 portion will be executed. If, however, a positive treatment effect is observed in the Phase 2b IA, participants in the Phase 2b portion will continue to undergo study procedures through Week 52 and may then enter the (optional) Phase 2b OLE phase as planned (provided they consent to do so and remain eligible for study participation).

The primary efficacy analysis for the Phase 2b IA will be conducted in the same fashion as described in Section “Primary Efficacy Endpoint”, but with the following changes:
1. The analysis variable will be changed from Baseline in the corresponding endpoints at Week 24.
2. All available data collected up to Week 24 will be included in a Mixed Model with Repeated Measures (MMRM). Data from study visits after Week 24 will be excluded from the primary MMRM model for the IA.

If, however, the IA or primary analysis for the Phase 2b portion shows a positive treatment effect for AMX0035, and if the totality of the data—i.e., efficacy, safety, and biomarker-related endpoint data—is supportive of continuing the study, participants enrolled in the Phase 2b portion will continue on either their double-blind study drug assignment or on open-label AMX0035 (as applicable), and the Phase 3 study portion will be initiated (with additional details to be provided in an amendment to the protocol).

At the time of the preplanned IA for the Phase 2b study portion, a committee internal to the Sponsor (for which the membership and details will be described in a separate document) will review the unblinded results and make the decision to either continue or stop the study.

The full details of the IA will be described in the SAP.

### Phase 3 Analyses

If the decision is made to proceed with the Phase 3 portion of this study, the design of that portion—to include the study objectives, endpoints, and procedures—is planned to be identical to that of the Phase 2b portion. Any changes introduced in the Phase 3 study portion will be provided in a future amendment to the protocol.

The primary objective of the Phase 3 portion of this study will be to compare AMX0035 against placebo by assessing the change from baseline in PSPRS at Week 52. The null hypothesis is H₀: CFB_{AMX0035}−CFB_{placebo}=0, where CFB_{AMX0035} is the change from baseline in PSPRS at Week 52 in the AMX0035 treatment group and CFB_{placebo} is the change from baseline in PSPRS at Week 52 in the placebo treatment group. The alternative hypothesis is defined as Ha: CFB_{AMX0035}−CFB_{placebo}>0.

If the Phase 3 portion of the study is ultimately conducted, a preplanned IA will be performed when approximately 40% of the total information from that portion becomes available (i.e., when 40% of the participants have completed 52 weeks of study treatment, withdrawn from study treatment, or died). Efficacy stopping based on Gamma spending (Hwang 1990) with parameter −4 and futility stopping based on Rho spending function (Kim 1987) with parameter −1.5 will be implemented for the Phase 3 portion in order to control type I error.

The primary analysis for the Phase 3 study portion will be performed when all participants in that portion have completed 52 weeks of study treatment, have withdrawn from study treatment, or have died. Formal hypothesis testing will be performed and a p-value will be produced for the Phase 3 efficacy analyses.

The final Phase 3 analysis will be performed after the Phase 3 study portion is considered complete. This analysis will focus on overall survival.

There is no planned adjustment for multiple comparisons or multiplicity, as statistical testing will be performed in a prespecified and sequential order to control the type I error. At the primary analysis for the Phase 3 study portion, statistical testing will be performed on the primary endpoint at a two-sided significance alpha level of 0.05. Only if significance is achieved for the primary endpoint, statistical testing will be performed on the secondary endpoint, in the order of PSPRS Score, then MDS-UPDRS Part II Score, sequentially, at the same alpha level.

If significance is not achieved for the prior endpoint, no formal statistical testing will be performed for the subsequent endpoints.

The design of the Phase 2b and Phase 3 study portions is planned to be identical, and key elements of the planned efficacy analyses (including population, model specification, and strategy for intercurrent events), safety analyses, and other analyses for the Phase 3 portion-if that portion is ultimately conducted-will therefore be aligned with those for Phase 2b. Any changes made to data analysis considerations and/or statistical methodology for the Phase 3 study portion, as applicable, will be provided in an amendment to the protocol.

Additional details will be provided in the Phase 3 SAP, as applicable.

### Start and End of Study Definitions

The start of the study is defined as the date of site activation, where the Investigator is given the authority to recruit participants. The date on which the first participant signs a study-specific ICF will be defined as the start of recruitment for this study. A participant is considered enrolled when the study-specific ICF is signed.

The date on which the last participant completes survival follow-up will be defined as the end of the study.

### Interim Analysis for Sample Size Re-estimation or Adjustment

A preplanned interim analysis (IA) may be performed when approximately 50% of the total information becomes available. At IA, a “promising zone design” will be utilized allowing sample size re-adjustment based on the conditional power. Details will be specified in the Independent Data Monitoring Committee (IDMC) charter and the SAP.

The final study analysis, which is to be focused on overall survival, is planned to be performed 5 years after the last participant has completed study treatment.

### Data Analysis Considerations

The Intention-to-Treat (ITT) Population will comprise all randomized participants regardless of whether or not study treatment was administered. This population will be based on the treatment to which the participant was randomized.

The Safety Population will consist of all participants who receive at least one dose of study treatment and will be based on the actual treatment received if this differs from that to which the participant was randomized.

The primary efficacy analysis will be conducted after all participants have completed the double-blind phase of the study, have withdrawn from study treatment, or have died.

### Key Elements of the Analysis Plan

Any deviations from or additions to the original analysis plan described in the protocol will be documented in the SAP and clinical study report.

All data up to the time of study completion/withdrawal from study will be included in the analysis, regardless of duration of treatment.

Demographic and Baseline characteristics will be summarized.

### Analysis for Efficacy

Efficacy analyses will be performed using the ITT population. Unless stated otherwise, all tests of treatment effects will be conducted at a two-sided significance level of 0.05.

The primary efficacy analyses will be for comparative efficacy of AMX0035 to placebo in the Treatment Period of the double-blind phase (including all data through Week 52 for participants discontinuing study treatment during the double-blind phase).

Details of the efficacy analyses for the OLE phase will be provided in the SAP.

The Phase 2b portion of the study is for estimation purposes only, and no formal hypothesis testing will be conducted. Point estimates and 95% confidence intervals of the primary and secondary endpoints will be provided.

As detailed above, efficacy stopping and futility stopping will be implemented for the Phase 3 study portion (if performed) in order to control type I error. Additionally, formal hypothesis testing will be performed, and a p-value produced, for analysis of efficacy in Phase 3 (as applicable).

### Primary Efficacy Endpoint

The primary analysis targets a treatment policy strategy by assessing treatment effect (change from baseline in PSPRS score) at Week 52. Change from baseline to each post-baseline assessment up to and including Week 52 will be analyzed using the likelihood-based Mixed Model with Repeated Measures (MMRM).

The estimand for the primary analysis is defined as follows:
Target Population: Intention-to-Treat (ITT)
Variable: Change from Baseline in PSPRS score at Week 52
Treatments: AMX0035 (1 gram of taurursodiol and 3 grams of sodium phenylbutyrate) or Placebo twice per day for 52 weeks
Summary measure: Difference in the mean change from baseline PSPRS score at Week 52
Strategy for Intercurrent events: Intercurrent events will be handled with the following strategies:
   Non-death treatment discontinuations (non-death): Treatment policy All available data up to Week 52 will be utilized regardless of treatment discontinuation. No imputations will be made for unobserved PSPRS data prior to Week 52.
   Death up to Week 52: Treatment policy

Death is assumed not informative to treatment effects on PSPRS score. No imputation will be made for PSPRS score after death.

Modeling details will be provided in SAP.

Change from baseline at Week 52 in the total PSPRS Score will be analyzed using MMRM with baseline value of the total PSPRS Score, stratification factor of region (North America, Europe), treatment, visit (categorical) and treatment*visit interaction as covariates. Unstructured will be used as the covariance structure. If lack of convergence is observed, the following structures will be tested in order and the first that converges will be used: Heterogeneous Toeplitz, Heterogeneous first order autoregressive, Heterogeneous compound symmetry, Toeplitz, first order autoregressive, compound symmetry. Restricted maximum likelihood (REML) will be used to estimate model parameters and the Kenward-Roger approximation will be used to estimate denominator degrees of freedom.

### Secondary Efficacy Endpoints

Analyses of the secondary efficacy endpoints will be conducted in the same fashion as the primary analysis described in Section “Primary Efficacy Endpoint”, with the following changes to the MMRM model specifications:
1. Analysis variable will be change from baseline at Week 52 in the corresponding secondary efficacy endpoint.
2. In the covariates of the MMRM, the baseline value of the corresponding secondary efficacy endpoint will be used.

MMRM analysis will be applied to each efficacy variable with repeated measurements. The analysis of secondary endpoints will be provided detailed in the SAP.

### Sensitivity Analysis

For the Phase 3 portion of the study only, to evaluate the robustness of the efficacy results, sensitivity analysis of the primary endpoint will be conducted to consider different missing data mechanisms, such as missing data imputed by multiple imputation under the assumption of missing not at random (MNAR). Sensitivity analysis will be conducted only if a statistically significant result is demonstrated in the primary efficacy analysis described above in Section “Primary Efficacy Endpoint”). Details of implementing the missing data imputations will be included in the Phase 3 SAP.

### Safety Analysis

The safety analyses will be performed on the Safety Population.

Adverse events (AEs) will be summarized by Medical Dictionary for Regulatory Activities (MedDRA) System Organ Class (SOC) and Preferred Term (PT) in tables that present the number of participants with at least one AE and the number of AEs.

Withdrawals, abnormal laboratory tests, vital signs, and use of concomitant medications will be listed.

Descriptive statistics for observed and changes from baseline for laboratory parameters and vital signs will be provided.

### Adverse Events

AE analyses, including the analysis of AEs, SAEs, and other significant AEs, will be summarized descriptively for the Safety Population. A TEAE is defined as an AEs wherefore which the onset date is on or after the study treatment start date. All AEs will be coded according to System Organ Class (MedDRA SOC) and Preferred Term (PT) using MedDRA.

Summary tables showing the number of participants and percent within each category will be generated for each of the following types of AEs:
All TEAEs
SAEs
Deaths
AEs leading to treatment interruption or treatment discontinuation and/or withdrawal
Fatal AEs
AEs by maximum severity level
Treatment-related AEs

All AEs will be tabulated and listed.

### Laboratory Parameters

Laboratory parameters (chemistry panel) will be summarized by visit and treatment group. Summary of laboratory values and change from baseline to the Week 24 and Week 52/EOT visits using mean, median, standard deviation, minimum, and maximum will be provided.

For all the laboratory tests that are gradable by NCI CTCAE v5.0, summaries of grade change from baseline grade will be provided by each scheduled visit and worst-case post-baseline. For laboratory tests that are not gradable by NCI CTCAE v5.0, summaries of changes from baseline with respect to normal range by each scheduled visit and worst-case post-baseline will be generated.

### Other Analyses

### Pharmacokinetic Analysis

Blood samples will be collected from the first 110 participants randomized in the study in order to assess exposure to PB, TURSO, and their respective metabolites following multiple doses of AMX0035. PK analysis may occur after the study is completed.

### Biomarker Analysis

Biomarker analyses will be described in the SAP.

### Medical Resource Utilization

Medical resource utilization analyses will be described in a separate healthcare resource utilization (HCRU) analysis plan.

### REFERENCES

1. Chen Y H, DeMets D L, Lan K K. Increasing the sample size when the unblinded interim result is promising. Stat Med. 2004-4-15; 23(7):1023-38.
2. Clinical Trials Facilitation and Coordination Group (CTFG). Recommendations related to contraception and pregnancy testing in clinical trials, version 1.1. 2020. Available from: https://www.hma.eu/fileadmin/dateien/Human_Medicines/01-About_HMA/Working_Groups/CTFG/2020_09_HMA_CTFG_Contraception_guidance_Vers ion_1.1_updated.pdf. Accessed 26-4-2024.
3. Columbia University Medical Center. Columbia-Suicide Severity Rating Scale (C—SSRS). Available from: http//www.cssrs.columbia.edu/. Accessed 26-4-2024.
4. Coyle-Gilchrist I T, Dick K M, Patterson K, et al. Prevalence, characteristics, and survival of frontotemporal lobar degeneration syndromes. Neurology. 2016; 86:1736-43.
5. Cuadrado-Tejedor M, Ricobaraza A L, Torrigo R, et al. Phenylbutyrate is a multifaceted drug that exerts neuroprotective effects and reverses the Alzheimer's disease-like phenotype of a commonly used mouse model. Curr Pharm Des. 2013; 19(28):5076-84.
6. Dionisio P A, Amaral J D, Ribeiro M F, et al. Amyloid-O pathology is attenuated by tauroursodeoxycholic acid treatment in APP/PS1 mice after disease onset. Neurobiol Aging. 2015 Jan; 36(1):228-40.
7. Folstein S E, Folstein S E, McHugh P R. “Mini-mental state.” A practical method for grading the cognitive state of patients for the clinician. J Psychiatry Res. 1975; 12(3):189-98.
8. Goetz C G, Fahn S, Martinez-Martin P, et al. Movement Disorder Society-sponsored revision of the Unified Parkinson's Disease Rating Scale (MDS-UPDRS): Process, format, and clinimetric testing plan. Mov Disord. 2007; 22(1):41-7.
9. Golbe L I. Progressive supranuclear palsy. Semin Neurol. 2014; 34(2):151-9.
10. Golbe L I, Ohman-Strickland P A. A clinical rating scale for progressive supranuclear palsy. Brain. 2007; 130(Pt 6):1552-65.
11. Hall D A, Forjaz M J, Golbe L I, et al. Scales to assess clinical features of progressive supranuclear palsy: MDS Task Force Report. Movement Disorders Clinical Practice. 2015; 2(2):127-34.
12. Höglinger G U, Respondek G, Stamelou M, et al. Clinical diagnosis of progressive supranuclear palsy: The Movement Disorder Society Criteria. Mov Disord. 2017; 32(6): 853-64.
13. Hwang I K, Shih W J, De Cani J S. Group sequential designs using a family of type I error probability spending functions. Stat Med. 1990 Dec; 9(12):1439-45.
14. Kim K, DeMets D L. Design and Analysis of Group Sequential Tests Based on the Type I Error Spending Rate Function. Biometrika. 1987; 74:149-54.
15. Lau D H W, Hartopp N, Welsh N J, et al. Disruption of ER-mitochondria signalling in
16. fronto-temporal dementia and related amyotrophic lateral sclerosis. Cell Death Dis. 2018; 9:327.
17. Lo A C, Callaerts-Vegh Z, Nunes A F, et al. Tauroursodeoxycholic acid (TUDCA) supplementation prevents cognitive impairment and amyloid deposition in APP/PS1 mice. Neurobiol Dis. 2013 Feb; 50:21-9.
18. Martinez-Martin P, Gil-Nagel A, Gracia L M, et al. Unified Parkinson's disease rating scale characteristics and structure. The Cooperative Multicentric Group. Mov Disord. 1994; 9(1): 76-83.
19. Mehta C R, Pocock S J. Adaptive increase in sample size when interim results are promising: A practical guide with examples. Stat Med. 2011; 30(28):3267-84.
20. Nasreddine Z S, Phillips N A, Bédirian V, et al. The Montreal Cognitive Assessment, MoCA: a brief screening tool for mild cognitive impairment. J Am Geriatr Soc. 2005; 53(4):695-9.
21. National Cancer Institute Common Terminology Criteria for Adverse Events (CTCAE) (Version 5.0). Available from: https://ctep.cancer.gov/protocoldevelopment/electronic_applications/docs/ctcae_v5_quick_ref erence_5×7.pdf. Accessed 26-4-2024.
22. Nunes A F, Amaral J D, Lo A C, et al. TUDCA, a bile acid, attenuates amyloid precursor protein processing and amyloid-O deposition in APP/PSi mice. Mol Neurobiol. 2012 Jun; 45(3):440-54.
23. Ricobaraza A, Cuadrado-Tejedor M, Perez-Mediavilla A, et al. Phenylbutyrate ameliorates cognitive deficit and reduces tau pathology in an Alzheimer's disease mouse model. Neuropsychopharmacology. 2009 Jun; 34(7):1721-32.
24. Rodrigues C M, Solá S, Sharpe J C, et al. Tauroursodeoxycholic acid prevents Bax-induced membrane perturbation and cytochrome C release in isolated mitochondria. Biochemistry. 2003(A) Mar; 42(10):3070-80.
25. Schwab R S, England A C. Projection technique for evaluating surgery in Parkinson's disease. In: Gillingham F J, Donaldson M C, Eds. Third synopsium on Parkinson's disease. Edinburgh. Livingston. 1969:152-7.
26. Shao Z, Janse E, Visser K, et al. What do verbal fluency tasks measure?Predictors of verbal fluency performance in older adults. Front Psychol. 2014; 5:772.
27. Stamelou M, Respondek G, Giagkou N, et al. Evolving concepts in progressive supranuclear palsy and other 4-repeat tauopathies. Nat Rev Neurol. 2021; 17:601-20.
28. Suaud L, Miller K, Panichelli A E, et al. 4-Phenylbutyrate stimulates Hsp70 expression through the Elp2 component of elongator and STAT-3 in cystic fibrosis epithelial cells. J Biol Chem. 2011-12-30; 286(52):45083-92.
29. Takigawa H, Kitayama M, Wada-Isoe K, et al. Prevalence of progressive supranuclear palsy in Yonago: change throughout a decade. Brain Behav. 2016; 6(12):e00557.
30. Wiley JC, Pettan-Brewer C, Ladiges W C. Phenylbutyric acid reduces amyloid plaques and rescues cognitive behavior in AD transgenic mice. Aging Cell. 2011 Jun; 10(3):418-28.
31. Williams D R, Lees A J. Progressive supranuclear palsy: clinicopathological concepts and diagnostic challenges. Lancet Neurol. 2009; 8(3):270-9.
32. Wright J M, Zeitlin P L, Cebotaru L, et al. Gene expression profile analysis of 4-phenylbutyrate treatment of 1B3-1 bronchial epithelial cell line demonstrates a major influence on heat-shock proteins. Physiol Genomics. 2004 Jan; 16(2):204-11.
33. Zhou W, Bercury K, Cummiskey J, et al. Phenylbutyrate up-regulates the DJ-1 protein and protects neurons in cell culture and in animal models of Parkinson disease. J Biol Chem. 2011 Apr; 286(17):14941-51.

1. A method of treating at least one symptom of progressive supranuclear palsy (PSP) in a human subject, the method comprising administering to the human subject a pharmaceutically effective amount of a combination of taurursodiol (TURSO) and sodium phenylbutyrate.

2. A method of treating a human subject having progressive supranuclear palsy (PSP), the method comprising administering to the human subject a pharmaceutically effective amount of a combination of TURSO and sodium phenylbutyrate.

3. A method of slowing progressive supranuclear palsy (PSP) progression in a human subject having one or more symptoms of PSP, the method comprising administering to the subject a pharmaceutically effective amount of a combination of TURSO and sodium phenylbutyrate.

4. A method of increasing survival time of a human subject having one or more symptoms of progressive supranuclear palsy (PSP), the method comprising administering to the human subject a pharmaceutically effective amount of a combination of TURSO and sodium phenylbutyrate.

5. A method of decreasing the level of total CSF tau, decreasing the level of CSF phospho-tau, increasing CSF Aβ₁₋₄₂/Aβ₁₋₄₀, or increasing the level of CSF 8-OHDG in a human subject having one or more symptoms of progressive supranuclear palsy (PSP), the method comprising administering to the human subject a pharmaceutically effective amount of a combination of TURSO and sodium phenylbutyrate.

6. (canceled)

7. A method comprising administering to a human subject at risk for developing progressive supranuclear palsy (PSP) a pharmaceutically effective amount of a combination of TURSO and sodium phenylbutyrate.

8. The method of claim 7, wherein the subject is determined to be at risk for developing progressive supranuclear palsy (PSP) by evaluating a level of a biomarker in a biological sample obtained from the subject.

9.-10. (canceled)

11. A method of decreasing the level of CSF YKL-40, decreasing the level of Ptpn1, or increasing the CSF ratio of 33 kDa tau to 55 kDa tau in a human subject having one or more symptoms of PSP, the method comprising administering to the human subject a pharmaceutically effective amount of a combination of TURSO and sodium phenylbutyrate.

12. The method of claim 1, wherein the human subject:
(a) has possible or probable PSP (Steele-Richardson-Olszewski Syndrome) according to MDS 2017 criteria;
(b) has had PSP symptoms for less than 5 years;
(c) has a PSP Rating Score total score of less than 40; or
(d) has a Mini-Mental Score Exam of greater than or equal to 24.

13. (canceled)

14. The method of claim 1, wherein the TURSO and the sodium phenylbutyrate are administered once a day or twice a day.

15. The method of claim 1, wherein the TURSO is administered to the subject at a dose of about 5 mg/kg to about 100 mg/kg and/or the sodium phenylbutyrate is administered to the subject at a dose of about 10 mg/kg to about 400 mg/kg.

16. (canceled)

17. The method of claim 1, wherein the TURSO is administered at an amount of about 0.5 to about 5 grams per day and/or the sodium phenylbutyrate is administered at an amount of about 0.5 grams to about 10 grams per day.

18. (canceled)

19. The method of claim 1, wherein the administering comprises administering to the subject about 1 gram of TURSO and about 3 grams of sodium phenylbutyrate once a day or twice a day.

20. The method of claim 1, comprising administering to the subject about 1 gram of TURSO once a day and about 3 grams of sodium phenylbutyrate once a day for about 14 days or more, followed by administering to the subject about 1 gram of TURSO twice a day and about 3 grams of sodium phenylbutyrate twice a day.

21. The method of claim 1, further comprising assessing one or more outcome measure after about 24 weeks of administering TURSO and sodium phenylbutyrate to the subject, determining an improvement in one or more of the outcome measures, and continuing administration of TURSO and sodium phenylbutyrate.

22. The method of claim 21, wherein the outcome measure is Progressive Supranuclear Palsy (PSP) Rating Score, Movement Disorder Society-Unified Parkinson's Disease Rating Scale Part IL, Schwab and England Activities of Daily Living Scale, EuroQuality of Life, Zarit Burden Interview, Clinical Global Impression of Severity and Change, Mini-Mental State Exam, Montreal Cognitive Assessment, Columbia-Suicide Severity Rating Scale, one or more plasma biomarker, or one or more CSF biomarker.

23. A method of treating or improving at least one symptom of progressive supranuclear palsy (PSP) in a human subject, the method comprising:
assessing a baseline PSP Rating Scale score prior to initial administration of TURSO and sodium phenylbutyrate to the human subject,
administering to the human subject about 1 gram of taurursodiol (TURSO) once a day and about 3 grams of sodium phenylbutyrate once a day for at least three weeks, followed by administering to the human subject about 1 gram of TURSO twice a day and about 3 grams of sodium phenylbutyrate twice a day,
assessing a second PSP Rating Scale at about 24 weeks after initial administration of TURSO and sodium phenylbutyrate to the human subject,
determining that the second PSP Rating Scale is higher than the baseline PSP Rating Scale, and
continuing to administer to the human subject about 1 gram of TURSO twice a day and about 3 grams of sodium phenylbutyrate twice a day, thereby treating or improving at least one symptom of progressive supranuclear palsy (PSP).

24. The method of claim 1, wherein the TURSO and the sodium phenylbutyrate are administered orally.

25. The method of claim 1, wherein the TURSO and the sodium phenylbutyrate are formulated as a single powder formulation.

26.-27. (canceled)

28. The method of claim 1, wherein the human subject is about 18 years or older.

29. (canceled)
